(19) United States
(12) Patent Application Publication
(10) Pub. No.: US 2009/0324618 A1
(43) Pub. Date: Dec. 31, 2009

(54) NOVEL SIGNATURE SELF RENEWAL GENE EXPRESSION PROGRAMS

(76) Inventors: Scott A. Armstrong, Wayland, MA (US); Andrei V. Krivtsov, Malden, MA (US)

Correspondence Address: POLSINELLI SHUGHART PC, 700 W. 47TH STREET, SUITE 1000, KANSAS CITY, MO, 64112-1802, US

(21) Appl. No.: 12/294,195

(22) PCT Filed: Mar. 26, 2007
(86) PCT No.: PCT/US07/07448

§ 371 Date: Apr. 6, 2009

Related U.S. Application Data
(60) Provisional application No. 60/785,532, filed on Mar. 24, 2006.
(60) Provisional application No. 60/852,021, filed on Oct. 16, 2006.

(51) Int. Cl. A61K 39/395; C12Q 1/68; A61K 31/7088; A61K 31/7105; C12N 5/06; G01N 33/574

(52) U.S. Cl. 424/178.1; 435/6; 514/44 A; 435/325; 435/7.23

(57) ABSTRACT

The present invention relates to compounds and methods which are useful in molecular investigations of target genes, as well as their encoded RNAs and protein, belonging to signature self renewal programs in leukemia and/or cancer stem cells. Data herein shows that leukemia stem cells can be generated from committed progenitors without widespread reprogramming of gene expression, and wherein a leukemia self-renewal associated signature is activated in the process.

### CROSS REFERENCE TO RELATED APPLICATIONS

This application claims the benefit of U.S. Provisional Application No. 60/785,532, filed on Mar. 24, 2006 and U.S. Provisional Application No. 60/852,021, filed on Oct. 16, 2006.

### GOVERNMENT RIGHTS

This work was supported by the National Cancer Institute (Grant No. K08 CA92551-05). The U.S. Government has certain rights in this invention.

### TECHNICAL FIELD

The present invention provides methods, and therapeutic, diagnostic, and preventative compounds/reagents for use in the identification and treatment of leukemia and cancer. Furthermore, the present invention relates to compounds and methods which are useful in molecular investigations of target genes, as well as their encoded RNAs and protein, belonging to signature self renewal programs in leukemia and/or cancer stem cells.

### BACKGROUND OF THE INVENTION

Leukemia and other cancers possess a rare population of cells capable of self-renewal. Eradication of these cancer stem cells is likely necessary for long-term cancer-free survival. If targeted leukemia and cancer cell therapy is to be successful, the extent to which cancer stem cells resemble normal tissue stem cells must be resolved. Both normal and cancer stem cells are capable of self renewal. Self renewal is the process by which cells divide to produce progeny that retain developmental, survival and proliferative potential. Postnatally, self-renewal properties are normally restricted to tissue stem cells, and the numbers of cells capable of self-renewal are tightly regulated. The importance of this tight regulation is highlighted by the fact that uncontrolled proliferation of cells capable of limitless self-renewal is a characteristic of leukemias and other cancers. 1. While it had been thought that all neoplastic cells in a tumor were capable of self-renewal, accumulating evidence supports the notion that leukemias and most other cancers are composed of a mixture of neoplastic cells, only a fraction of which possess this property. 1-3.

Therefore, what is needed is a method which can effectively eradicate and specifically target those neoplastic cells within a tumor. Eradication of these so-called cancer stem cells is likely to be a critical part of any successful anti-cancer therapy, and may explain why conventional cancer therapies are often effective in reducing tumor burden, but only rarely curative. Significant interest has focused on the identification and characterization of this population, and the possibility that differences between normal and cancer stem cells might be exploited to develop highly effective, minimally toxic therapies.

Gene expression profiling is a very useful technique for characterizing the nature and state of cells, including disease progression, pharmacological response, as well as biological phenomena such as growth and development. Data generated from such profiling often point to specific sets of genes and gene pathways (“gene expression signature(s)”) that are specifically associated with the state, change in state, disease diagnosis or prognosis, and drug induced responses. The identification of these gene expression signatures often provides critical information relating to the biological state of a cancer cell, for example, or a cell's response to a potential therapeutic agent. The discovery of gene expression signatures is proving to be a powerful tool for disease diagnosis and drug discovery. For example, in the area of oncology, microarray analysis is being broadly applied toward the diagnosis and classification of a host of different cancers.

A detailed understanding of normal hematopoiesis, and characterization of normal hematopoietic stem and progenitor cells has aided the study of leukemia stem cells (LSC). 4,5. The initial studies that demonstrated the presence of LSC provided strong support for the cancer stem cell hypothesis. 6. These studies demonstrated that human leukemia cells expressing the early hematopoietic marker CD34 could be separated into CD38 positive and negative populations, and that only the CD34⁺/CD38⁻ population was capable of initiating leukemia in non-obese diabetic/severe combined immunodeficient (NOD/SCID) mice. 6,7. As normal stem cells are found in the CD34⁺/CD38⁻ population, these data suggest that leukemia stem cells are phenotypically similar to normal hematopoietic stem cells (HSC). However, recent data from murine models suggest that leukemia stem cell development may be initiated from either HSC or more committed progenitors that have no inherent self-renewal properties. 8,9. Furthermore, in vitro studies suggest LSC may be found in progenitor populations in chronic myelogenous leukemias that have progressed to blast crisis. 10. However, no study has identified a sufficiently enriched population to determine if LSC must be phenotypically similar to normal HSC or if they can retain the identity of committed progenitors. The successful development of targeted leukemia and cancer therapies having high therapeutic indices depends upon whether the therapy can effectively target the LSC. The more closely a LSC resembles a normal HSC, the more difficult it may be to develop efficacious and specific therapies.

A need exists for an accurate determination of gene expression and phenotypic analyses in order to characterize the cellular identity of LSCs and HSCs, and to further develop targeted therapies based upon these studies.

A major obstacle in cancer stem cell research is the general difficulty in determining which populations of cells to study. A need remains for methods directed to identifying population of cells that have the greatest potential to develop cancer stem cell activity. One such method would identify the cancer cells with the highest expression of a self-renewal stem cell signature.

A tumor with a greater self-renewal capability is generally thought to be more difficult to treat. Therefore, the potential for using data derived from gene expression analyses may benefit prediction models directed to responsiveness to therapy and therapy outcome. Indeed, multiple studies have demonstrated the potential for gene expression signatures to predict response to therapy and outcome in cancer. However, there has not yet been a study which has identified a sufficiently pure population of cancer stem cells to define a true self-renewal associated signature.

### SUMMARY OF THE INVENTION

The present invention provides methods, and therapeutic, diagnostic, and preventative compounds/reagents for use in the identification and treatment of leukemia and cancer. Furthermore, the present invention relates to compounds and methods which are useful in molecular investigations of target genes, as well as their encoded RNAs and protein, belonging to signature self renewal gene expression programs in leukemia and/or cancer stem cells. These compounds are, for example, stable nucleic acid agents, which may be used to knockdown or down regulate target genes; antibodies, which may be used to target specific leukemia and/or cancer stem cell antigens; nucleic acid oligonucleotides, for use as probes in the identification of normal, cancer and/or leukemia stem cells; and small molecule drugs, biologic and non-biologic. The nucleic acids of the present invention may be easily modified to adjust for single-nucleotide polymorphisms which may be reflected in the targeted DNA or RNA molecule(s).

The present invention is based upon studies, illustrated herein, which have identified, for example, a range of gene expression differences between stem cells, for example normal hematopoietic stem cells; committed progenitor cells; and leukemia stem cells. These studies demonstrate that leukemia stem cells can be generated from committed progenitors without widespread reprogramming of gene expression, and wherein a leukemia self-renewal associated signature is activated in the process. The gene expression signatures identified herein are correlated with clinical parameters to identify potentially new biomarkers and therapeutic targets of leukemia and/or cancer. Committed progenitor cells are well known in the art.

As described herein, a progenitor-derived leukemia stem cell (LSC) can possess an immunophenotype and a global gene expression profile most similar to a normal committed progenitor cell. However, a self-renewal associated program normally expressed in hematopoietic stem cells (HSC) is activated during the transformation from committed progenitor to LSC (see FIG. 1). This self-renewal program is described herein, as well as individual genes in the program that define pathways and genetic networks for the conversion of a normal progenitor cell to a leukemia cell.

In one embodiment, the invention relates to a method of treating a leukemia, for example a mixed lineage leukemia, comprising administering to an individual in need thereof a therapeutic amount of an agent that inhibits the activity of a gene product which is encoded by a self-renewal associated signature gene. Examples of self-renewal associated signature genes as identified herein are shown in Table 2. In a preferred embodiment, the gene product is encoded by a gene selected from the group consisting of HoxA6, HoxA7, HoxA9, HoxA10, Mef2c, EphA7, Runx2, Nln, Tcf4, Meis1, Galgt, Fdx1, Itf-2, and HoxA5.

In another embodiment, the invention relates to a method of treating a leukemia, for example a mixed lineage leukemia, comprising administering to an individual in need thereof a therapeutic amount of an agent that inhibits the expression of a gene identified as a self-renewal associated signature gene. In a preferred embodiment, the gene may be selected from the group consisting of HoxA6, HoxA7, HoxA9, HoxA10, Mef2c, EphA7, Runx2, Nln, Tcf4, Meis1, Galgt, Fdx1, Itf-2, and HoxA5.

In another embodiment, the present invention relates to the identification of individual genes that can be targeted for therapy. For example, the below-demonstration that shRNA mediated inhibition of Mef2c leads to decreased LSC proliferation and survival provides support for this idea. Furthermore, individual genes were tested as potential therapeutic targets in human leukemia. Expression of Mef2c, EphA7, RunxZ, Nln, Tcf4, Meis1, HoxA9, Galgt, Fdx1, HoxA10, and HoxA5 was suppressed in MLL-rearranged human leukemia cell lines. This suppression inhibited leukemia cell proliferation and survival, thus making these genes, for example, potential therapeutic targets. The self-renewal program described herein is likely to be at least partially active in all cancer stem cells; therefore, the therapeutics described herein for leukemia may be of benefit in other cancers.

The invention further relates to methods of diagnosing leukemias, comprising determining a gene expression profile of a gene expression product present in at least one or more stem cells, for example hematopoietic stem cells or leukemia stem cells, wherein the gene expression profile is correlated with the gene expression profile of a progenitor cell; for example, a leukemia-like granulocyte-macrophage progenitor cell.

The invention further relates to methods of diagnosing leukemias, comprising determining a gene expression profile of mRNA from one or more genes, wherein the mRNA is isolated from one or more cells of an individual, for example mononuclear blood cells and bone marrow cells; and comparing the obtained gene expression profile to a gene expression profile of a control leukemia sample, wherein the gene expression profile of the cell from the individual is indicative of a leukemia.

Methods for diagnosing a leukemia in a first tissue sample of an individual are disclosed, wherein such methods comprise, for example, the steps of (a) determining the expression profile of one or more self-renewal associated signature genes of cancer-like progenitor cells in the first tissue sample, and (b) comparing the pattern or level of expression profile observed with the pattern or level of expression of the same genes in a second tissue sample comprising committed progenitor cells, wherein increased expression of the one or more self-renewal associated signature genes in the first tissue sample indicates leukemia. The cancer-like progenitor cells include leukemia cells, for example. The first and second tissues include, but are not limited to, epithelial tissue, connective tissue, osseous tissue, vascular tissue, blood, muscle tissue, nervous tissue, and cartilage. In such methods, the first and the second tissue samples do not have to be of the same tissue. For example, the first and the second tissue may be of different types or come from different individuals (or, alternatively, the first and second tissue may be of different types but come from the same individual). Examples of committed progenitor cells include, but are not limited to, granulocyte-macrophage progenitors, common myeloid progenitors (CMP), and megakaryocyte erythroid progenitors (MEP). Table 2 is a list of self-renewal associated signature genes as identified herein. A self-renewal gene expression signature refers to a set of genes that are expressed in, for example, hemapoietic stem cells and leukemia stem cells, but not normal, non-self renewing, progenitor cells. In a preferred embodiment of the present invention a signature set of genes can consist of one or more genes. It another preferred embodiment, a signature set of genes consists of between about 5 and 15 genes. For example, a signature set of genes may consist of 11 genes; for example, HOXA9, HOXA10, MEF2c, HOXA5, Meis1, ITF2, MYLK, RUNX2, PELI1, LAPTM4b, and STAU2. It another preferred embodiment, a signature set of genes consists of between about 15 and 30 genes. In yet another embodiment, a signature set of genes consists of between about 50 and 100 genes. In other embodiments, the signature sets of genes may consist of between 100 and 200 genes, between 200 and 300 genes, or between 300 and 400 genes, for example. In the methods described herein the one or more self-renewal associated signature genes may be selected from the group consisting of HOXA9, HOXA10, MEF2c, HOXA5, Meis1, ITF2, MYLK, RUNX2, PELI1, LAPTM4b, and STAU2. In one example, if the one or more of the genes is expressed at higher levels in the first tissue sample than in the corresponding one or more genes in the second tissue sample, this is indicative of the presence of leukemia or cancer.

Methods for targeted therapeutic treatment of leukemia or cancer cells are also disclosed herein. These methods comprise, for example, administering to a patient in need thereof an effective amount of a therapeutic agent that targets one or more self-renewal signature genes or gene products (RNA or protein, for example) expressed in the leukemia or cancer cells. The therapeutic may comprise a drug conjugated to an immunoglobulin or aptamer that specifically recognizes an epitope on a protein encoded by the one or more self-renewal signature genes. Alternatively, the therapeutic may be a polynucleotide capable of binding to and reducing the expression of a nucleic acid encoding one or more of the self-renewal signature genes. Such therapeutics may reduce the in vivo expression of the one or more self-renewal signature genes. The polynucleotide can be an effective amount of a siNA complementary to a target 3′UTR mRNA encoded by one or more self-renewal signature genes; for example, a miRNA which can direct interference of the target mRNA (often resulting in mRNA degradation and/or translational repression). Such siNAs may be administered by a route selected from the group consisting of oral, intravenous, intramuscular, and intrapulmonary.

Transformed cell lines are also disclosed herein, wherein the cell line expresses an MLL-AF9 fusion protein. The cell line may be a committed progenitor selected from the group consisting of granulocyte-macrophage progenitors (GMP); common myeloid progenitors (CMP), and megakaryocyte erythroid progenitors (MEP). For example, see FIG. 15.

Methods for detecting the presence of leukemia stem cells in a tissue sample are disclosed, wherein such methods comprise reacting a first tissue sample with one or more antibodies that specifically bind to one or more gene products of the self-renewal associated signature genes, wherein detecting the antibody-gene product complex indicates the presence of leukemia stem cells. Methods well known in the art may be employed to isolate and purify the leukemia stem cells and/or the gene product. One such method is immunoprecipitation. In a preferred embodiment of the present invention, the antibody is specific for EPHA7.

As briefly described above, the present invention provides new cell populations and lines which express MLL-AF9 fusion proteins encoded by the t(9;11)(p22;q23) found in human acute myelogenous leukemia (AML). To produce such cells, MLL-AF9 is expressed in myeloid progenitor or hematopoietic stem cells and the cells are immediately injected into recipient mice. Upon leukemia development, IL-7⁻ lin⁻ Sca-1⁻ c-Kit⁺ CD34⁺ FcγRII/III⁺ (L-GMP), are isolated. Cell lines are subsequently generated by passage of the cells in culture in the presence of interleukin-3. These cell lines, for example L-GMP, can be used in any method directed to investigating the molecular mechanisms of leukemia, treatments for leukemia, screens for therapeutic agents, etc.

The present invention provides new nucleic acid molecules which regulate targeted gene expression and/or mRNA stability. Furthermore, the present invention relates to compounds and methods which are useful in molecular investigations of these target genes, and their encoded RNAs; and, additionally, in the diagnosis, prevention, and therapy of leukemia and/or cancer. These compounds are stable nucleic acid agents which may be used to knockdown or down regulate target genes. For example, one such nucleic acid agent is a siRNA as herein described. The nucleic acids of the present invention may be easily modified to adjust for single-nucleotide polymorphisms which may be reflected in the targeted DNA or RNA molecule(s).

The instant invention also features small nucleic acid molecules, such as short interfering nucleic acid (siNA), short interfering RNA (siRNA), double-stranded RNA (dsRNA), micro-RNA (miRNA), and short hairpin RNA (shRNA) molecules and methods used to modulate the expression of target leukemia and cancer genes. The siNAs of the present invention, for example miRNAs, regulate gene expression via target RNA transcript cleavage/degradation or translational repression of the target messenger RNA (mRNA). miRNAs are natively expressed, typically as final 19-22 non-translated RNA products. miRNAs exhibit their activity through sequence-specific interactions with the 3′ untranslated regions (UTR) of target mRNAs. These endogenously expressed miRNAs from hairpin precursors which are subsequently processed into a miRNA duplex, and further into a “mature” single stranded miRNA molecule. This mature miRNA guides a multiprotein complex, miRISC, which identifies target 3′ UTR regions of mRNAs based upon their complementarity to the mature miRNA.

siRNAs are exogenously expressed small RNAs that modulate gene expression through similar mechanisms as miRNA.

Endogenously produced miRNAs differ from siRNAs in terms of their synthesis, but both can direct cleavage or degradation of homologous targets, or repress the translation of partially complementary targets.

A siNA of the invention can be unmodified or chemically-modified. A siNA of the instant invention can be chemically synthesized, expressed from a vector or enzymatically synthesized. The instant invention also features various chemically-modified synthetic short interfering nucleic acid (siNA) molecules capable of modulating gene expression or activity in cells by RNA interference (RNAi). The use of chemically-modified siNA improves various properties of native siNA molecules through, for example, increased resistance to nuclease degradation in vivo and/or through improved cellular uptake. Furthermore, siNA having multiple chemical modifications may retain its RNAi activity. The siNA molecules of the instant invention provide useful reagents and methods for a variety of therapeutic, diagnostic, target validation, genomic discovery, genetic engineering, and pharmacogenomic applications.

As will be further described below, the present invention is also directed to methods for targeted therapeutic treatment of leukemia or cancer cells. These methods comprise, for example, administering to a patient in need thereof an effective amount of a therapeutic that targets one or more self-renewal signature gene products expressed in the leukemia or cancer cells. The therapeutic agent may comprise a drug conjugated to an immunoglobulin or aptamer that specifically recognizes an epitope on a protein encoded by the one or more self-renewal signature genes. Furthermore, the therapeutic may reduce in vivo expression of the one or more self-renewal signature genes. Aptamers include, but are not limited to DNA oligonucleotides (DNA aptamers), RNA oligonucleotides (RNA aptamers), peptide aptamers comprising, for example, a short variable peptide domain attached at both ends to a protein scaffold.

### FIGURES AND DRAWINGS

FIG. 1: Model for development of leukemia stem cells from committed progenitors. The herein described MLL-AF9 fusion can induce serial replating activity and leukemia from GMP. The signature induced soon after MLL-AF9 expression includes a subset of genes that are part of a larger self-renewal associated signature that accumulates in the developing leukemia stem cell. However, the leukemia stem cell retains a global identity that is most similar to the GMP from which it arose.

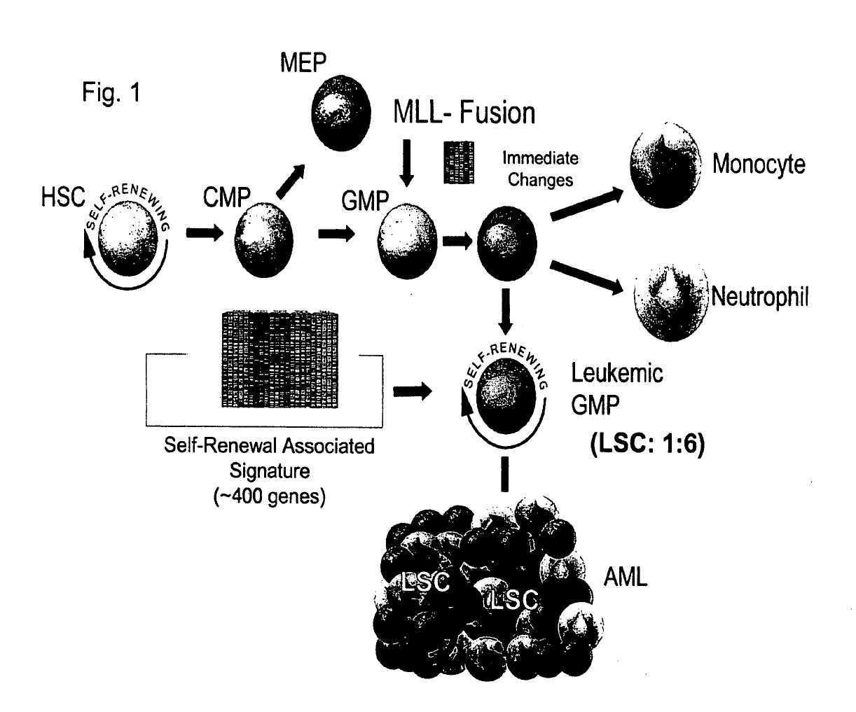

FIG. 2: MLL-AF9 induces leukemia from GMP. (A) Normal IISC and progenitor populations including the IL7R⁻ Lin⁻ Sca-1⁺ c-kit⁺ HSC-enriched population, IL-7R⁻ Lin⁻ Sca-1⁻ c-Kit⁺ CD34⁺ FcγRII/III^{lo} common myeloid progenitors (CMP), IL-7R⁻ Lin⁻ Sca-1⁻ c-Kit⁺ CD34⁺ FcγRII/III^{high} granulocyte macrophage progenitors (GMP), and IL-7R⁻ Lin⁻ Sca-1⁻ c-Kit⁺ CD34⁻ FcγRII/III⁻ megakaryocyte erythroid progenitors (MEP) were isolated as previously described and 5×10⁴-1×10⁵ GMP were transduced with either MSCV-GFP control or MLL-AF9-GFP retroviruses. Forty hours later the GFP positive cells were isolated for further experiments. 5,000 GMP transduced with the indicated retroviruses were plated in cytokine-supplemented methylcellulose. Every seven days the number of colonies was determined and 5,000 cells re-plated in methylcellulose. Only the MLL-AF9 transduced cells produced colonies past the 3^{rd} week.

FIG. 3: L-GMP are enriched for CFC-blast. Bone marrow from leukemic mice was assessed for the presence of myeloid progenitors. The rare non-leukemic Lin⁻ GFP⁻ Kit⁺ population contains cells possessing the immunophenotype of CMP and GMP. The GFP⁺ leukemia cells represent >90% of bone marrow cells and possess Lin⁻ IL7R⁻ Scal⁻ GFP⁺ Kit⁺ FcγRII/III⁺ CD34⁺ L-GMP. L-GMP were prospectively purified for injection to secondary recipients or gene expression analysis. The experiment was repeated 3 times with similar results. The bar graph illustrates a comparison of colony forming activity between L-GMP, Lin−, Kit−, and lin+ cells. L-GMP were sorted from two mice that developed AML after transduction of normal GMP with MLL-AF9. 250 cells were subsequently cultured in methylcellulose supplemented with SCF, IL3 and 1L6. This experiment was repeated two times with cells isolated from 4 different mice with similar results.

FIG. 4: Leukemic-GMPs are enriched for leukemia stem cells. a, Survival curves for mice injected with limiting dilution of 5×10³, 500, 100, 20 or 4 L-GMP from a representative experiment is shown. The experiment was repeated 3 times with similar results. In total 22 mice were injected with 20 cells and all succumbed to AML, and 6 mice were injected with 4 cells with one succumbing to AML. b, Immunophenotypic analysis of myeloid progenitors from a leukemia generated by injection of 20 L-GMP shows recapitulation of the initial disease shown in FIG. 3.

FIG. 5: L-GMP differentiate upon in vitro propagation. Immunophenotypic analysis of L-GMP propagated in liquid culture (AKLG cells) supplemented with IL3 demonstrates the majority of cells have differentiated into a Lin+, Kit-population. Survival curves for 3 mice injected with 5,000 AKLG cells and 3 mice injected with 100,000 AKLG cells demonstrate the requirement for >5,000 AKLG cells to initiate leukemia.

FIG. 6: Characterization of leukemias generated by introduction of 20 L-GMP into secondary recipients. Histological analysis shows infiltration of larger cells with large nuclei similar to the leukemias in primary recipients. FACS analysis demonstrates leukemia cells are GFP+, Macl+, Grl+, CD3−, B220− similar to primary leukemias. MLL-Af9 initiates leukemia from committed progenitors. Progenitor analysis shown in FIGS. 1 and 2 further demonstrate the similarity between the primary and secondary leukemias.

FIG. 7: Progenitor-derived leukemia stem cells maintain progenitor identity and reactivate a self-renewal associated program. RNA was isolated from multiple independently isolated samples containing normal HSC, CMP, GMP, MEP, and L-GMP. L-GMP samples 1 and 2 were isolated from the bone marrow and spleen of a single mouse. Other L-GMP samples are from 4 separate mice. We routinely isolated RNA from 2-5×10⁴ cells, and performed two rounds of in vitro transcription as previously described¹⁸. All amplified RNA samples were labeled and hybridized to Affymetrix murine 430A 2.0 microarrays. a, Hierarchical clustering was performed using the 9,100 probe sets that passed a filter of max/min>2 and max−min>80. The data shows the gene expression relationship between each of the sorted samples. The leukemic GMPs arc most closely related to the GMP population from which they arose, but make up their own branch of the dendrogram, b, K-means clustering was performed to identify major gene expression clusters. The figure shows the probe sets that passed a filter of max/min−3 and max−min=100. Signature 1a demonstrates genes that show increased expression in the normal GMP population and even greater expression in the L-GMP. Signature Ib is the inverse of 1a. Signature 2 shows a group of genes that are highly expressed in HSC population and show lower level expression in all other groups including L-GMP. Signature 3a shows genes that demonstrate high-level expression in the HSC population, decreased expression in committed progenitors and re-activation of high-level expression in L-GMP. Signature 3b is the inverse of 3a. c, The top 50 probe sets for genes that show elevated expression in the HSC population and L-GMP are shown. Permutation testing demonstrates 420 probe sets (363 genes) in this signature (p<0.001). The full signatures can be found at http://www.broad.mit.edu/personal/twomey/armstrong/. d, GSEA was performed to assess the 363-gene self-renewal associated signature in the L-GMP compared to AKLG cells and normal GMP. The analysis demonstrated significant enrichment of the signature in the L-GMP (p<0.001), thus demonstrating a decrease in the self-renewal associated signature in cells derived from L-GMP that have lost leukemia stem cell activity, e, CD48 expression in L-GMP and GMP was assessed by flow cytometry and compared to an isotype control antibody. The L-GMP demonstrated homogenous expression at a level lower than found on GMP.

FIG. 8: Hox Genes and Mef2c play a role in L-GMP: a, 5,000 GMP transduced with the indicated retroviruses were plated in cytokine-supplemented methylcellulose. Every seven days the number of colonies was determined, and 5000 cells re-plated in methylcellulose. HoxA6, HoxA7, HoxA9, and HoxAlO transduced GMP could be re-plated for >4 weeks. HoxA5 and control transduced cells produced no colonies past the 2^{nd} week, b, GMP transduced with Mef2c were subjected to serial replating as above. The colony forming activity was unchanged, but there were consistently 5-fold more cells in the Mef2c-transduced colonies as compared to control cells. The data represent 5 independent experiments.

FIG. 9. Mef2c shRNAs inhibit colony forming activity. L-GMP were transduced with lentiviruses expressing either a control shRNA (luciferase) or either of two Mef2c directed shRNA (Mef2c-F6, Mef2c-F7) and 1000 cells were plated in methylcellulose supplemented with cytokines and puromycin. After selection with puromycin, Mef2c RNA levels were assessed by real-time PCR. L-GMP colonies were counted on day 5 after transduction with either an empty lentivirus (pLKO:puro), or viruses expressing either luciferase, Mef2c-F6 or Mef2-fl shRNAs.

FIG. 10. Mef2c shRNAs decrease leukemogenic potential. Individual colonies isolated on day 5 after transduction with either a control lentivirus (n=30) or Mef2c-F6 shRNA (n=20) were injected into sublethally irradiated syngeneic recipients, and monitored for the development of AML. A survival curve for both groups is shown demonstrating a significant difference in survival when the experiment was terminated at day 112 (p=0.015). On day 112, 1 mouse in each group contained GFP positive cells and were thus counted as having developed AML.

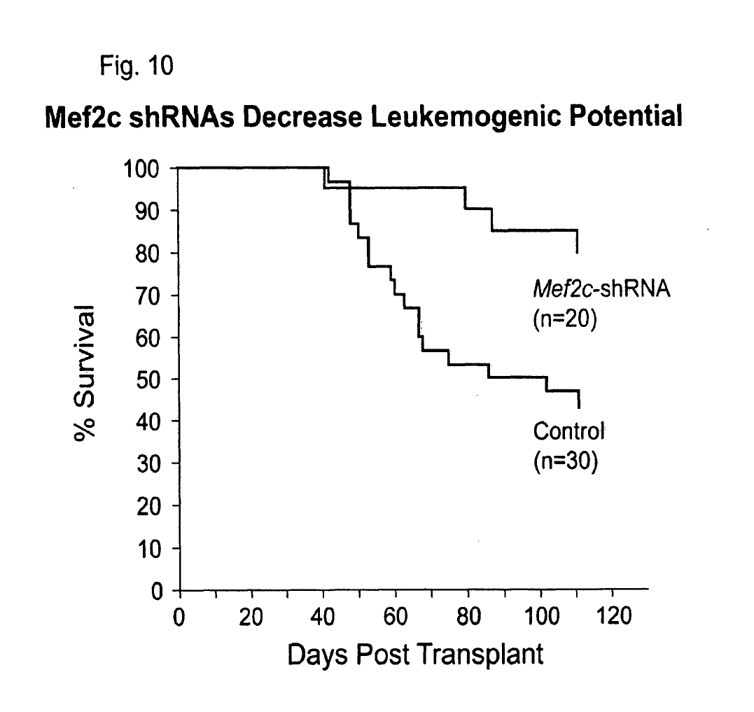

FIG. 11. Effects of HOXA9 knockdown in 17 human leukemia cells lines. Analysis of HOXA9 expression and of survival.

FIG. 12. Effects of HOXA9 knockdown in 17 human leukemia cell lines. Correlation between HOXA9 expression and survival after knockdown.

FIG. 13. Effects of HOXA9 knockdown in primary AML patient samples. AML patient samples consisted of 6 MLL-rearranged AML and 4 MLL-germline AML. Efficient HOXA9 knockdown was confirmed by Q-PCR.

FIG. 14. In vivo effects of HOXA9 knockdown—Post mortem analysis at day 14. The results show an 80% knockdown at day 0 and equal expression at day 14. SEMK2 cells that escaped HOXA9 knockdown show survival advantage in vivo.

FIG. 15. MLL-AF9 transforms human GMP.

### DETAILED DESCRIPTION OF THE INVENTION

The present invention provides methods, and therapeutic, diagnostic, and preventative compounds/reagents for use in the identification and treatment of leukemia and cancer. Furthermore, the present invention relates to compounds and methods which are useful in molecular investigations of target genes, as well as their encoded RNAs and protein, belonging to signature self renewal programs in leukemia and/or cancer stem cells. These compounds are, for example, stable nucleic acid agents, which may be used to knockdown or down regulate target genes; antibodies, which may be used to target specific leukemia and/or cancer stem cell antigens; nucleic acid oligonucleotides, for use as probes in the identification of normal, cancer and/or leukemia stem cells; and small molecule drugs, biologic and non-biologic. The nucleic acids of the present invention may be easily modified to adjust for single-nucleotide polymorphisms which may be reflected in the targeted DNA or RNA molecule(s).

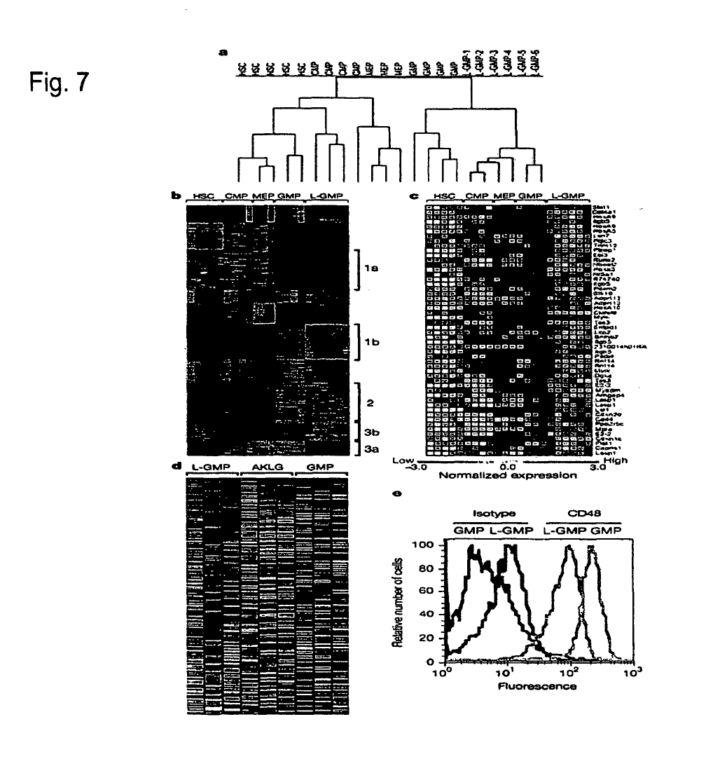

Herein described is an example of a signature self renewal program. This program is a group of genes which is (1) highly expressed in the hematopoietic stem cell-populations (HSC), (2) shows decreased expression in committed progenitors, but have re-activated high-level expression in a leukemia-like granulocyte-macrophage progenitor (L-GMP) population (signature 3a in FIG. 7), and the inverse (signature 3b in FIG. 7). These 363 genes that are more highly expressed in HSC and LSC (a self-renewal associated signature) represent only a subset of the approximately 1137 genes normally highly expressed in HSC compared to committed progenitors (FIG. 7).

The invention further relates to a method of identifying a compound for use in treating a leukemia, for example a mixed lineage leukemia, or cancer comprising determining a gene expression profile of a gene expression product from at least one gene of a signature self renewal program from one or more cells of an individual with leukemia or cancer; administering a test agent to the individual; determining a gene expression profile of a gene expression product from at least one gene from a signature self renewal program from one or more cells from the individual; and comparing the two gene expression profiles, wherein if the gene expression profile from the individual after administration of the agent is correlated with effective treatment of the leukemia or cancer, the test agent is a therapeutic agent. In one embodiment, the disease is a mixed lineage leukemia, and a decrease in the expression of the gene selected from the group consisting of HOXA9, HOXA10, MEF2c, HOXA5, Meis1, ITF2, MYLK, RUNX2, PELI1, LAPTM4b, and STAU2, is indicative of effective treatment of the leukemia. In another embodiment, the gene expression profiles compared prior to and after administration of the test agent consist of one or more of the same signature self renewal genes.

The invention also relates to a method for evaluating drug candidates for their effectiveness in treating leukemia, for example a mixed lineage leukemia, or a cancer comprising contacting a cell sample or lysate thereof with a candidate compound, wherein the cell; and detecting an alteration of a gene expression profile of a gene expression product from at least one signature self renewal gene from the cell sample or lysate thereof, wherein a compound that decreases the gene expression profile of at least one signature self renewal gene which is increased in the leukemia, it is a compound for use in treating leukemia. In a preferred embodiment, the disease is mixed lineage leukemia, and the signature self renewal gene is selected from the group consisting of HOXA9, HOXA10, MEF2c, HOXA5, Meis1, ITF2, MYLK, RUNX2, PELI1, LAPTM4b, and STAU2.

The invention further relates to a method of identifying a compound for use in treating leukemia, comprising contacting a cell sample or lysate thereof with a candidate compound, wherein the cell; and detecting an alteration of a gene expression profile of a gene expression product from at least one signature self renewal gene from the cell sample or lysate thereof, wherein a compound that increases the gene expression profile of at least one signature self renewal gene which is decreased in leukemia is a compound for use in treating leukemia.

The invention further relates to a method of identifying a compound for use in treating leukemia, comprising contacting a cell sample or lysate thereof with a candidate compound; and detecting an alteration of a gene expression profile of a gene expression product from at least one signature self renewal gene from the cell sample or lysate thereof, wherein a compound that increases the gene expression profile of at least one signature self renewal gene which is decreased in leukemia, is a compound for use in treating leukemia.

In another aspect, the invention relates to a method of identifying a compound that modulates (increases or decreases) the biological activity of one signature self renewal gene.

In still another aspect, the invention features a method of identifying a compound that decreases the biological activity of a signature self renewal gene product having increased expression in a leukemia, for example AML. The method comprises contacting the one signature self renewal gene expression product with a candidate compound under conditions suitable for activity of the one signature self renewal gene expression product; and assessing the biological activity level of the one signature self renewal gene expression product. A candidate compound that decreases the biological activity level of the signature self renewal gene product relative to a control is a compound that decreases the biological activity of the one signature self renewal gene expression product having increased expression in the leukemia. In one embodiment, the method is carried out in a cell or animal. In another embodiment, the method is carried out in a cell-free system. The invention further relates to a method of identifying a compound for use in treating, for example, mixed lineage leukemia, acute lymphoblastic leukemia, or acute myelogenous leukemia, comprising determining a gene expression profile of a gene expression product from at least one signature self renewal gene from one or more cells of an individual with a leukemia; administering a test agent to the individual; determining a gene expression profile of a gene expression product from at least one signature self renewal gene from one or more cells from the individual; and comparing the two gene expression profiles, wherein if the gene expression profile from the individual after administration of the agent is correlated with effective treatment of leukemia, the test agent is a therapeutic agent. In one embodiment, the disease is mixed lineage leukemia, and a decrease in the expression of the one signature self renewal gene selected from the group consisting of HOXA9, HOXA10, MEF2c, HOXA5, Meis1, ITF2, MYLK, RUNX2, PELI1, LAPTM4b, and STAU2, is indicative of effective treatment of mixed lineage leukemia. In another embodiment, the gene expression profiles compared prior to and after administration of the test agent consist of one or more of the same signature self renewal genes.

The invention also relates to a method for evaluating drug candidates for their effectiveness in treating mixed lineage leukemia, acute lymphoblastic leukemia, or acute myelogenous leukemia, comprising contacting a cell sample or lysate thereof with a candidate compound; and detecting an alteration of a gene expression profile of a gene expression product from at least one signature self renewal gene from the cell sample or lysate thereof, wherein a compound that increases the gene expression profile of at least one signature self renewal gene which is decreased in mixed lineage leukemia, acute lymphoblastic leukemia, or acute myelogenous leukemia is a compound for use in treating mixed lineage leukemia, acute lymphoblastic leukemia, or acute myelogenous leukemia.

The invention further relates to a method of identifying a compound for use in treating leukemia, comprising contacting a cell sample or lysate thereof with a candidate compound; and detecting an alteration of a gene expression profile of a gene expression product from at least one signature self renewal gene from the cell sample or lysate thereof, wherein a compound that decreases the gene expression profile of at one signature self renewal gene which is increased in the leukemia is a compound for use in treating leukemia. In one embodiment, the disease is mixed lineage leukemia, and a decrease in the expression of the one signature self renewal gene selected from the group consisting of HOXA9, HOXA10, MEF2c, HOXA5, Meis1, ITF2, MYLK, RUNX2, PELI1, LAPTM4b, and STAU2, is indicative of effective treatment of mixed lineage leukemia.

The invention further relates to a method of identifying a compound for use in treating leukemia, comprising contacting a cell sample or lysate thereof with a candidate compound; and detecting an alteration of a gene expression profile of a gene expression product from at least one signature self renewal gene from the cell sample or lysate thereof, wherein a compound that increases the gene expression profile of at least one signature self renewal gene which is decreased in the leukemia, is a compound for use in treating acute lymphoblastic leukemia.

In another aspect, the invention relates to a method of identifying a compound that modulates (increases or decreases) the biological activity of a signature self renewal gene.

In still another aspect, the invention features a method of identifying a compound that decreases the biological activity of a signature self renewal gene expression product having increased expression in a leukemia or cancer. The method comprises contacting the signature self renewal gene expression product with a candidate compound under conditions suitable for activity of the signature self renewal gene product; and assessing the biological activity level of the signature self renewal gene expression product. A candidate compound that decreases the biological activity level of the signature self renewal gene expression product relative to a control is a compound that decreases the biological activity of the signature self renewal gene expression product having increased expression in leukemia. In one embodiment, the method is carried out in a cell or animal. In another embodiment, the method is carried out in a cell-free system. In still another embodiment the signature self renewal gene expression product is selected from the gene expression products encoded by the genes in Table 2. In one embodiment, the disease is mixed lineage leukemia, and a decrease in the activity level of the one signature self renewal gene product selected from the group consisting of HOXA9, HOXA10, MEF2c, HOXA5, Meis1, ITF2, MYLK, RUNX2, PELI1, LAPTM4b, and STAU2, is indicative of effective treatment of mixed lineage leukemia.

In another aspect, the invention features a method of identifying a compound that increases the biological activity of a signature self renewal gene product having decreased expression in leukemia. The method comprises contacting the signature self renewal gene product with a candidate compound under conditions suitable for biological activity of the signature self renewal gene product; and assessing the biological activity level of signature self renewal gene product. A candidate compound that increases the biological activity level of the signature self renewal gene product relative to a control is a compound that increases the biological activity of the signature self renewal gene product having decreased expression in leukemia.

In one embodiment, the method is carried out in a cell or animal. In another embodiment, the method is carried out in a cell-free system. In still another embodiment the signature self renewal gene product is selected from the gene expression products encoded by the genes in Table 2. In other embodiments, screens can be carried out for compounds that further increase the expression of a gene or the biological activity of a gene expression product already overexpressed in leukemia, or that further decrease the expression of a gene or the biological activity of a gene expression product already underexpressed in leukemia. These compounds can be identified according the screening methods described herein. These compounds should be avoided during treatment regimens for leukemia.

In another aspect, the invention features a method of identifying a compound that increases the biological activity of a signature self renewal gene product having decreased expression in leukemia. The method comprises contacting the signature self renewal gene product with a candidate compound under conditions suitable for biological activity of the signature self renewal gene product; and assessing the biological activity level of the signature self renewal gene product. A candidate compound that increases the biological activity level of the signature self renewal gene product relative to a control is a compound that increases the biological activity of the signature self renewal gene product having decreased expression in leukemia.

In one embodiment, the method is carried out in a cell or animal. In another embodiment, the method is carried out in a cell-free system. In other embodiments, screens can be carried out for compounds that further increase the expression of a gene or the biological activity of a gene expression product already overexpressed in leukemia, or that further decrease the expression of a gene or the biological activity of a gene expression product already underexpressed in leukemia. These compounds can be identified according the screening methods described herein. These compounds should be avoided during treatment regimens for leukemia.

In yet another aspect of the present invention, leukemia stem cells of the present invention may be used to generate antibodies against gene products of genes belonging to the self renewal signature program and/or to products of genes that do not belong to the self renewal signature program.

### DEFINITIONS

As used herein, a “gene expression signature” refers generally to a group of genes that are determined to be differentially expressed after comparison of two or more gene expression profiles obtained from cells that are presumed to differ in some biologically important function. A gene expression signature can be specifically associated with, inter alia, a cell state, a change in a cell state, disease diagnosis or prognosis, and/or drug induced cell responses. For example, a “self-renewal gene expression signature” refers to a set of genes that are expressed in hemapoietic stem cells and leukemia stem cells, but not in normal, non-self renewing, progenitor cells. For example, these sets of genes may be highly expressed in hemapoietic stem cells and leukemia stem cells, but expressed at a lower level in non-self renewing, progenitor cells.

As used herein, a stem cell is an unspecialized cell that is capable of replicating or self renewing itself and developing into specialized cells of a variety of cell types. For example, a hematopoietic stem cell may produce a second generation stem cell and a neuron.

A progenitor cell (also known as a precursor cell) is unspecialized or has partial characteristics of a specialized cell that is capable of undergoing cell division and yielding two specialized cells. For example, a myeloid progenitor/precursor may undergo cell division to yield two specialized cells (a neutrophil and a monocyte). Progenitor cells of the present invention include, but are not limited to, common myeloid progenitors (CMP), granulocyte macrophage progenitors (GMP), and megakaryocyte erythroid progenitors (MEP).

A polynucleotide can be delivered to a cell to express an exogenous nucleotide sequence, to inhibit, eliminate, augment, or alter expression of an endogenous nucleotide sequence, or to affect a specific physiological characteristic not naturally associated with the cell. The polynucleotide can be a sequence whose presence or expression in a cell alters the expression or function of cellular genes or RNA. A delivered polynucleotide can stay within the cytoplasm or nucleus apart from the endogenous genetic material. Alternatively, DNA can recombine with (become a part of) the endogenous genetic material. Recombination can cause DNA to be inserted into chromosomal DNA by either homologous or non-homologous recombination.

A polynucleotide-based gene expression inhibitor comprises any polynucleotide containing a sequence whose presence or expression in a cell causes the degradation of or inhibits the function, transcription, or translation of a gene in a sequence-specific manner. Polynucleotide-based expression inhibitors may be selected from the group comprising: siRNA, microRNA, interfering RNA or RNAi, dsRNA, ribozymes, antisense polynucleotides, and DNA expression cassettes encoding siRNA, microRNA, dsRNA, ribozymes or antisense nucleic acids. SiRNA comprises a double stranded structure typically containing 15 to 50 base pairs and preferably 19 to 25 base pairs and having a nucleotide sequence identical or nearly identical to an expressed target gene or RNA within the cell. A siRNA may be composed of two annealed polynucleotides or a single polynucleotide that forms a hairpin structure. MicroRNAs (miRNAs) are small noncoding polynucleotides, about 22 nucleotides long that direct destruction or translational repression of their mRNA targets. Antisense polynucleotides comprise sequence that is complimentary to a gene or mRNA. Antisense polynucleotides include, but are not limited to: morpholinos, 2′-O-methyl polynucleotides, DNA, RNA and the like. The polynucleotide-based expression inhibitor may be polymerized in vitro, recombinant, contain chimeric sequences, or derivatives of these groups. The polynucleotide-based expression inhibitor may contain ribonucleotides, deoxyribonucleotides, synthetic nucleotides, or any suitable combination such that the target RNA and/or gene is inhibited.

Polynucleotides may contain an expression cassette coded to express a whole or partial protein, or RNA. An expression cassette refers to a natural or recombinantly produced polynucleotide that is capable of expressing a sequence. The cassette contains the coding region of the gene of interest along with any other sequences that affect expression of the sequence of interest. An expression cassette typically includes a promoter (allowing transcription initiation), and a transcribed sequence. Optionally, the expression cassette may include, but is not limited to, transcriptional enhancers, non-coding sequences, splicing signals, transcription termination signals, and polyadenylation signals. An RNA expression cassette typically includes a translation initiation codon (allowing translation initiation), and a sequence encoding one or more proteins. Optionally, the expression cassette may include, but is not limited to, translation termination signals, a polyadenosine sequence, internal ribosome entry sites (IRES), and non-coding sequences. The polynucleotide may contain sequences that do not serve a specific function in the target cell but are used in the generation of the polynucleotide. Such sequences include, but are not limited to, sequences required for replication or selection of the polynucleotide in a host organism.

A polynucleotide can be delivered to a cell to study gene function. Delivery of a polynucleotide to a cell can also have potential clinical applications. Clinical applications include treatment of muscle disorders or injury, circulatory disorders, endocrine disorders, immune modulation and vaccination, and metabolic disorders (Baumgartner et al. 1998, Blau et al. 1995, Svensson et al. 1996, Baumgartner et al. 1998, Vale et al. 2001, Simovic et al. 2001).

A transfection agent, or transfection reagent or delivery vehicle, is a compound or compounds that bind(s) to or complex(es) with oligonucleotides and polynucleotides, and enhances their entry into cells. Examples of transfection reagents include, but are not limited to, cationic liposomes and lipids, polyamines, calcium phosphate precipitates, polycations, histone proteins, polyethylenimine, polylysine, and polyampholyte complexes. For delivery in vivo, complexes made with sub-neutralizing amounts of cationic transfection agent may be preferred. Non-viral vectors includes protein and polymer complexes (polyplexes), lipids and liposomes (lipoplexes), combinations of polymers and lipids (lipopolyplexes), and multilayered and recharged particles. Transfection agents may also condense nucleic acids. Transfection agents may also be used to associate functional groups with a polynucleotide. Functional groups include cell targeting moieties, cell receptor ligands, nuclear localization signals, compounds that enhance release of contents from endosomes or other intracellular vesicles (such as membrane active compounds), and other compounds that alter the behavior or interactions of the compound or complex to which they are attached (interaction modifiers).

The term naked nucleic acids indicates that the nucleic acids are not associated with a transfection reagent or other delivery vehicle that is required for the nucleic acid to be delivered to a target cell.

“Inhibit” or “down-regulate” means that the expression of the gene, or level of RNAs or equivalent RNAs encoding one or more proteins or isoforms, or translation of RNAs, or activity of one or more proteins is reduced below that observed in the absence of the nucleic acid molecules of the invention. In one embodiment, inhibition or down-regulation with the presently described nucleic acid molecules preferably is below that level observed in the presence of an enzymatically inactive or attenuated molecule that is able to bind to the same site on the target RNA, but is unable to cleave or inhibit translation of that RNA. In another embodiment, inhibition or down-regulation with antisense oligonucleotides is preferably below that level observed in the presence of, for example, an oligonucleotide with scrambled sequence or with mismatches. In another embodiment, inhibition or down-regulation of a target gene with the nucleic acid molecule of the instant invention is greater in the presence of the nucleic acid molecule than in its absence.

By “up-regulate” is meant that the expression of the gene, or level of RNAs or equivalent RNAs encoding one or more proteins or isoforms, or translation of RNAs, or activity of one or more proteins is greater than that observed in the absence of the nucleic acid molecules of the invention. For example, the expression of a gene can be increased in order to treat, prevent, ameliorate, or modulate a pathological condition caused or exacerbated by an absence or low level of gene expression.

By “modulate” is meant that the expression of the gene, or level of RNAs or equivalent RNAs encoding one or more proteins or subunits, or translation of RNAs, or activity of one or more proteins or protein isoforms is up-regulated or down-regulated, such that the expression, level, or activity is greater than or less than that observed in the absence of the nucleic acid molecules of the invention.

By “gene” it is meant a nucleic acid that encodes an RNA, for example, nucleic acid sequences including but not limited to structural genes encoding a polypeptide.

“Complementarity” refers to the ability of a nucleic acid to form hydrogen bond(s) with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., enzymatic nucleic acid cleavage, antisense or triple helix inhibition. Determination of binding free energies for nucleic acid molecules is well known in the art (see, e.g., Turner et al., 1987, CSH Symp. Quant. Biol. LII pp. 123 133; Frier et al., 1986, Proc. Nat. Acad. Sci. USA 83:9373 9377; Turner et al., 1987, J. Am. Chem. Soc. 109:3783 3785). A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

By “RNA” is meant a molecule comprising at least one ribonucleotide residue. By “ribonucleotide” or “2′-OH” is meant a nucleotide with a hydroxyl group at the 2′ position of a β-D-ribo-furanose moiety.

Chemically synthesizing nucleic acid molecules with modifications (base, sugar and/or phosphate) that prevent their degradation by serum ribonucleases can increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; and Burgin et al., supra; all of these describe various chemical modifications that can be made to the base, phosphate and/or sugar moieties of the nucleic acid molecules herein). Modifications which enhance their efficacy in cells, and removal of bases from nucleic acid molecules to shorten oligonucleotide synthesis times and reduce chemical requirements are desired. (All these publications are hereby incorporated by reference herein).

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-fluoro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modification of nucleic acid molecules have been extensively described in the art (see Eckstein et al., International Publication PCT No. WO 92/07065; Perrault et al. Nature, 1990, 344, 565 568; Pieken et al. Science, 1991, 253, 314 317; Usman and Cedergren, Trends in Biochem. Sci., 1992, 17, 334 339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; Earnshaw and Gait, 1998, Biopolymers (Nucleic acid Sciences), 48, 39 55; Verma and Eckstein, 1998, Annu. Rev. Biochem., 67, 99 134; and Burlina et al., 1997, Bioorg. Med. Chem., 5, 1999 2010; all of the references are hereby incorporated in their totality by reference herein). Such publications describe general methods and strategies to determine the location of incorporation of sugar, base and/or phosphate modifications and the like into ribozymes without inhibiting catalysis, and are incorporated by reference herein. In view of such teachings, similar modifications can be used as described herein to modify the nucleic acid molecules of the instant invention.

While chemical modification of oligonucleotide internucleotide linkages with phosphorothioate, phosphorothioate, and/or 5′-methylphosphonate linkages improves stability, too many of these modifications can cause some toxicity. Therefore when designing nucleic acid molecules the amount of these internucleotide linkages should be minimized. The reduction in the concentration of these linkages should lower toxicity resulting in increased efficacy and higher specificity of these molecules.

Nucleic acid molecules having chemical modifications that maintain or enhance activity are provided. Such nucleic acid is also generally more resistant to nucleases than unmodified nucleic acid. Thus, in a cell and/or in vivo the activity can not be significantly lowered. Therapeutic nucleic acid molecules delivered exogenously are optimally stable within cells until translation of the target RNA has been inhibited long enough to reduce the levels of the undesirable protein. This period of time varies between hours to days depending upon the disease state. Nucleic acid molecules are preferably resistant to nucleases in order to function as effective intracellular therapeutic agents. Improvements in the chemical synthesis of RNA and DNA (Wincott et al., 1995 Nucleic Acids Res. 23, 2677; Caruthers et al., 1992, Methods in Enzymology 211,3 (incorporated by reference herein) have expanded the ability to modify nucleic acid molecules by introducing nucleotide modifications to enhance their nuclease stability as described above.

In one embodiment, the invention features antibodies that specifically bind a polypeptide, preferably an epitope, of a signature self renewal gene of the present invention (as determined, for example, by immunoassays, a technique well known in the art for assaying specific antibody-antigen binding). One such polypeptide is that of EPHA7, a molecule that is only expressed on the surface of leukemia cells. Antibodies of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, for example, anti-Id antibodies to antibodies of the invention), and epitope-binding fragments of any of the above.

The term “antibody,” as used herein, refers to immunoglobulin molecules and immunologically active portions of immunoglobulin molecules, and more specifically, molecules that contain an antigen binding site that specifically binds an antigen. The immunoglobulin molecules of the invention can be of any type (for example, IgG, IgE, IgM, IgD, IgA and IgY), and of any class (for example, IgG1, IgG2, IgG3, IgG4, IgA1 and IgA2) or subclass of an immunoglobulin molecule.

In one embodiment, the antibodies are antigen-binding antibody fragments and include, without limitation, Fab, Fab′ and F(ab′)₂, Fd, single-chain Fvs (scFv), single-chain antibodies, disulfide-linked Fvs (dsFv) and fragments comprising either a V_{L} or V_{H} domain. Antigen-binding antibody fragments, including single-chain antibodies, can comprise the variable region(s) alone or in combination with the entirety or a portion of one or more of the following: hinge region, CH1, CH2, and CH3 domains. Also included in the invention are antigen-binding fragments also comprising any combination of variable region(s) with a hinge region, CH1, CH2, and/or CH3 domains.

The antibodies of the invention may be from any animal origin including birds and mammals. Preferably, the antibodies are human, murine, donkey, sheep, rabbit, goat, guinea pig, hamster, horse, or chicken.

As used herein, “human” antibodies include antibodies having the amino acid sequence of a human immunoglobulin and include antibodies produced by human B cells, or isolated from human sera, human immunoglobulin libraries or from animals transgenic for one or more human immunoglobulins and that do not express endogenous immunoglobulins, as described in U.S. Pat. No. 5,939,598 by Kucherlapati et al., for example.

The antibodies of the present invention may be monospecific, bispecific, trispecific or of greater multispecificity. Multispecific antibodies may be specific for different epitopes of a polypeptide of the present invention or may be specific for both a polypeptide of the present invention as well as for a heterologous epitope, such as a heterologous polypeptide or solid support material.

Antibodies of the present invention may be described or specified in terms of the epitope(s) or portion(s) of a polypeptide of the present invention that they recognize or specifically bind. The epitope(s) or polypeptide portion(s) may be specified, for example, by N-terminal and/or C-terminal positions, or by size in contiguous amino acid residues. Antibodies that specifically bind any epitope or polypeptide encoded by a gene of the present invention, for example a self renewing signature gene, may also be excluded. Therefore, the present invention includes antibodies that specifically bind a polypeptide encoded by a gene of the present invention, and allows for the exclusion of the same.

The term “epitope,” as used herein, refers to a portion of a polypeptide which contacts an antigen-binding site(s) of an antibody or T cell receptor. Specific binding of an antibody to an antigen having one or more epitopes excludes non-specific binding to unrelated antigens, but does not necessarily exclude cross-reactivity with other antigens with similar epitopes.

Antibodies of the present invention may also be described or specified in terms of their cross-reactivity. Antibodies of the present invention may not display any cross-reactivity, such that they do not bind any other analog, ortholog, or homolog of a polypeptide of the present invention. Alternatively, antibodies of the invention can bind polypeptides with at least about 95%, 90%, 85%, 80%, 75%, 70%, 65%, 60%, 55%, or 50% identity (as calculated using methods known in the art) to a polypeptide encoded by a target gene of the present invention, for example an identified self renewal associated signature gene. Further included in the present invention are antibodies that bind polypeptides encoded by genes that hybridize to identified self renewal associated signature genes of the present invention under stringent hybridization conditions, as will be appreciated by one of skill in the art.

Antibodies of the present invention can also be described or specified in terms of their binding affinity to a polypeptide of the invention. Preferred binding affinities include those with a dissociation constant or Kd less than 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹M, 5×10⁻¹⁰ M, 10⁻¹⁰ M, 5×10⁻¹¹M, 10⁻¹¹ M, 5×10⁻¹² M, 10⁻¹² M, 5×10⁻¹³M, 10⁻¹³ M, 5×10⁻¹⁴M, 10⁻¹⁴ M, 5×10⁻¹⁵ M, and 10⁻¹⁵ M.

The invention also provides antibodies that competitively inhibit binding of an antibody to an epitope of a polypeptide of the invention, as determined by any method known in the art for determining competitive binding, for example, using immunoassays. In particular embodiments, the antibody competitively inhibits binding to the epitope by at least about 90%, 80%, 70%, 60%, or 50%.

Antibodies of the present invention can act as agonists or antagonists of polypeptides encoded by the self renewal associated signature gene of the present invention. For example, the present invention includes antibodies which disrupt interactions with the polypeptides encoded by the identified self renewal associated signature gene of the invention either partially or fully. The invention also includes antibodies that do not prevent binding, but prevent activation or activity of the polypeptide. Activation or activity (for example, signaling) may be determined by techniques known in the art. Also included are antibodies that prevent both binding to and activity of a polypeptide encoded by an identified self renewal associated signature gene. Likewise included are neutralizing antibodies.

Antibodies of the present invention may be used, for example, and without limitation, to purify, detect, and target the polypeptides encoded by the identified self renewal associated signature gene described herein, including both in vitro and in vivo diagnostic and therapeutic methods. For example, the antibodies have use in immunoassays for qualitatively and quantitatively measuring levels of the polypeptides in biological samples. See, for example, Harlow et al., Antibodies: A Laboratory Manual (Cold Spring Harbor Laboratory Press, 2nd ed. 1988).

As discussed in more detail below, the antibodies of the present invention may be used either alone or in combination with other compositions. The antibodies may further be recombinantly fused to a heterologous polypeptide at the N- and/or C-terminus or chemically conjugated (including covalent and non-covalent conjugations) to polypeptides or other compositions. For example, antibodies of the present invention may be recombinantly fused or conjugated to molecules useful as labels in detection assays, or effector molecules such as heterologous polypeptides, drugs, or toxins. The antibodies may also pertain to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes A variety of radionuclides are available for the production of radioconjugated antibodies. Examples include ²¹²Bi, ¹³¹I, ¹³¹In, ⁹⁰Y, and ¹⁸⁶Re. Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO 94/11026.

The antibodies of the invention include derivatives that are modified, for example, by the covalent attachment of any type of molecule to the antibody such that covalent attachment does not prevent the antibody from recognizing its epitope. For example, but not by way of limitation, the antibody derivatives include antibodies that have been modified, for example, by glycosylation, acetylation, pegylation, phosphorylation, amidation, derivatization by known protecting/blocking groups, proteolytic cleavage, or linkage to a cellular ligand or other protein. Any of numerous chemical modifications can be carried out by known techniques, including, but not limited to, specific chemical cleavage, acetylation, formylation, and metabolic synthesis of tunicamycin. Additionally, the derivative can contain one or more non-classical amino acids.

The antibodies of the present invention can be generated by any suitable method known in the art. Polyclonal antibodies to an antigen-of-interest can be produced by various procedures well known in the art. For example, a polypeptide of the invention can be administered to various host animals including, but not limited to, rabbits, mice, rats, or the like, to induce the production of sera containing polyclonal antibodies specific for the antigen. Various adjuvants can be used to increase the immunological response, depending on the host species, and include, but are not limited to, Freund's adjuvant (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and corynebacterium parvum. Such adjuvants are well known in the art.

Monoclonal antibodies can be prepared using a wide variety of techniques also known in the art, including hybridoma cell culture, recombinant, and phage display technologies, or a combination thereof. For example, monoclonal antibodies can be produced using hybridoma techniques as is known in the art and taught, for example, in Harlow et al., Antibodies: A Laboratory Manual (Cold Spring Harbor Laboratory Press, 2nd ed. 1988). The term “monoclonal antibody” as used herein is not necessarily limited to antibodies produced through hybridoma technology, but also refers to an antibody that is derived from a single clone, including any eukaryotic, prokaryotic, or phage clone.

Human antibodies are desirable for therapeutic treatment of human patients. These antibodies can be made by a variety of methods known in the art including phage display methods using antibody libraries derived from human immunoglobulin sequences. Human antibodies can also be produced using transgenic mice that are incapable of expressing functional endogenous immunoglobulins, but which can express human immunoglobulin genes. The transgenic mice are immunized with a selected antigen, for example, all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For a detailed discussion of this technology for producing human antibodies and human monoclonal antibodies and protocols for producing such antibodies, see, for example, PCT publications WO 98/24893; WO 96/34096; WO 96/33735; and U.S. Pat. Nos. 5,413,923; 5,625,126; 5,633,425; 5,569,825; 5,661,016; 5,545,806; 5,814,318; and 5,939,598.

In another embodiment, antibodies to the polypeptides encoded by the identified self renewal associated signature genes as described herein can, in turn, be utilized to generate anti-idiotype antibodies that “mimic” polypeptides of the invention using techniques well known to those skilled in the art. (See, for example, Greenspan & Bona, FASEB J. 7(5):437-444 (1989) and Nissinoff, J. Immunol. 147(8):2429-2438 (1991)). For example, antibodies that bind to and competitively inhibit polypeptide multimerization and/or binding of a polypeptide to a ligand can be used to generate anti-idiotypes that “mimic” the polypeptide multimerization and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a polypeptide encoded by an identified self renewal associated signature gene and/or to bind its ligands, and thereby block its biological activity.

The antibodies or fragments thereof of the present invention can be fused to marker sequences, such as a peptide to facilitate their purification. In one embodiment, the marker amino acid sequence is a hexa-histidine peptide, an HA tag, or a FLAG tag, as will be readily appreciated by one of skill in the art.

The present invention further encompasses antibodies or fragments thereof conjugated to a diagnostic or therapeutic agent. The antibodies can be used diagnostically, for example, to monitor the development or progression of a tumor as part of a clinical testing procedure to determine the efficacy of a given treatment regimen. Detection can be facilitated by coupling the antibody to a detectable substance. Examples of detectable substances include enzymes (such as, horseradish peroxidase, alkaline phosphatase, β-galactosidase, or acetylcholinesterase), prosthetic group (such as streptavidin/biotin and avidin/biotin), fluorescent materials (such as umbelliferone, fluorescein, fluorescein isothiocyanate, rhodamine, dichlorotriazinylamine fluorescein, dansyl chloride or phycoerythrin), luminescent materials (such as luminol), bioluminescent materials (such as luciferase, luciferin, and aequorin), radioactive materials (such as, ¹²⁵I, ¹³¹I, ¹¹¹In or ⁹⁹Tc), and positron emitting metals using various positron emission tomographies, and nonradioactive paramagnetic metal ions.

In an additional embodiment, an antibody or fragment thereof can be conjugated to a therapeutic moiety such as a cytotoxin, for example, a cytostatic or cytocidal agent, a therapeutic agent or a radioactive metal ion. A cytotoxin or cytotoxic agent includes any agent that is detrimental to cells. Examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (for example, daunorubicin (formerly daunomycin) and doxorubicin), antibiotics (for example, actinomycin, bleomycin, mithramycin, and anthramycin (AMC)), and anti-mitotic agents (for example, vincristine and vinblastine).

The conjugates of the invention can be used for modifying a given biological response, the therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator, a thrombotic agent or an anti-angiogenic agent, for example, angiostatin or endostatin; or, biological response modifiers such as, for example, lymphokines, interleukins, granulocyte macrophase colony stimulating factor (“GM-CSF”), granulocyte colony stimulating factor (“G-CSF”), or other growth factors.

Antibodies of the invention can also be attached to solid supports. These are particularly useful for immunoassays or purification of the target antigen. Such solid supports include, but are not limited to, glass, cellulose, silicon, polyacrylamide, nylon, polystyrene, polyvinyl chloride or polypropylene. Techniques for conjugating such therapeutic moiety to antibodies are well known in the art, see, for example, Amon et al., “Monoclonal Antibodies For Immunotargeting Of Drugs In Cancer Therapy”, in Monoclonal Antibodies And Cancer Therapy, Reisfeld et al. eds., pp. 243-56 (Alan R. Liss, Inc. 1985).

Alternatively, an antibody can be conjugated to a second antibody to form an antibody heteroconjugate as described by Segal in U.S. Pat. No. 4,676,980.

An antibody of the invention, with or without conjugation to a therapeutic moiety, administered alone or in combination with cytotoxic factor(s) and/or cytokine(s), can be used as a therapeutic.

Antisense antagonists of the present invention are also included. Antisense technology can be used to control gene expression through antisense DNA or RNA, or through triple-helix formation. Antisense techniques are discussed for example, in Okano, J., Neurochem. 56:560 (1991). The methods are based on binding of a polynucleotide to a complementary DNA or RNA. In one embodiment, an antisense sequence is generated internally by the organism, in another embodiment, the antisense sequence is separately administered (see, for example, O'Connor, J., Neurochem. 56:560 (1991)).

In one embodiment, the 5′ coding portion of an identified self renewal associated signature gene can be used to design an antisense RNA oligonucleotide from about 10 to 40 base pairs in length. Generally, a DNA oligonucleotide is designed to be complementary to a region of the gene involved in transcription thereby preventing transcription and the production of the receptor. The antisense RNA oligonucleotide hybridizes to the mRNA in vivo and blocks translation of the mRNA molecule into receptor polypeptide.

In one embodiment, the antisense nucleic acid of the invention is produced intracellularly by transcription from an exogenous sequence. For example, a vector or a portion thereof, is transcribed, producing an antisense nucleic acid of the invention. Such a vector contains the sequence encoding the antisense nucleic acid. The vector can remain episomal or become chromosomally integrated, as long as it can be transcribed to produce the desired antisense RNA. Vectors can be constructed by recombinant DNA technology and can be plasmid, viral, or otherwise, as is known to one of skill in the art.

Expression can be controlled by any promoter known in the art to act in the target cells, such as vertebrate cells, and preferably human cells. Such promoters can be inducible or constitutive and include, without limitation, the SV40 early promoter region (Bemoist and Chambon, Nature 29:304-310 (1981), the promoter contained in the 3′ long terminal repeat of Rous sarcoma virus (Yamamoto et al., Cell 22:787-797 (1980)), the herpes thymidine promoter (Wagner et al., Proc. Natl. Acad. Sci. U.S.A. 78:1441-1445 (1981)), and the regulatory sequences of the metallothionein gene (Brinster et al., Nature 296:3942 (1982)).

The antisense nucleic acids of the invention comprise a sequence complementary to at least a portion of an RNA transcript of an identified self renewal associated signature gene. Absolute complementarity, although preferred, is not required. A sequence “complementary to at least a portion of an RNA,” referred to herein, means a sequence having sufficient complementarity to be able to hybridize with the RNA, forming a stable duplex. The ability to hybridize will depend on both the degree of complementarity and the length of the antisense nucleic acid. Generally, the larger the hybridizing nucleic acid, the more base mismatches with the RNA it may contain and still form a stable duplex. One skilled in the art can ascertain a tolerable degree of mismatch by use of standard procedures to determine the melting point of the hybridized complex.

Oligonucleotides that are complementary to the 5′ end of the RNA, for example, the 5′ untranslated sequence up to and including the AUG initiation codon, are generally regarded to work most efficiently at inhibiting translation. However, sequences complementary to the 3′ untranslated sequences of mRNAs have been shown to be effective at inhibiting translation of mRNAs as well. Thus, oligonucleotides complementary to either the 5′- or 3′-non-translated, non-coding regions of a nucleotide sequence can be used in an antisense approach to inhibit mRNA translation. Oligonucleotides complementary to the 5′ untranslated region of the mRNA can include the complement of the AUG start codon. Antisense oligonucleotides complementary to mRNA coding regions can also be used in accordance with the invention. In one embodiment, the antisense nucleic acids are at least six nucleotides in length, and are preferably oligonucleotides ranging from about 6 to about 50 nucleotides in length. In other embodiments, the oligonucleotide is at least about 10, 17, 25 or 50 nucleotides in length.

The antisense oligonucleotides of the invention can be DNA or RNA, or chimeric mixtures, or derivatives or modified versions thereof, single-stranded or double-stranded. The oligonucleotide can be modified at the base moiety, sugar moiety, or phosphate backbone, for example, to improve stability of the molecule, hybridization, and the like. The oligonucleotide can include other appended groups such as peptides (for example, to target host cell receptors in vivo), or agents that facilitate transport across the cell membrane, or the blood-brain barrier, or intercalating agents.

The antisense oligonucleotide may comprise at least one modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, a-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, β-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including, but not limited to, arabinose, 2-fluoroarabinose, xylulose, and hexose.

In yet another embodiment, the antisense oligonucleotide comprises at least one modified phosphate backbone selected from the group including, but not limited to, a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier et al., Nucl. Acids Res. 15:6625-6641 (1987)). The oligonucleotide is a 2′-O-methylribonucleotide (Inoue et al., Nucl. Acids Res. 15:6131-6148 (1987)), or a chimeric RNA-DNA analog (Inoue et al., FEBS Lett. 215:327-330 (1987)).

Antisense oligonucleotides of the invention may be synthesized by standard methods known in the art, for example, by use of an automated DNA synthesizer.

Potential antagonists according to the invention also include catalytic RNA, or a ribozyme. Hammerhead ribozymes cleave mRNAs at locations dictated by flanking regions that form complementary base pairs with the target mRNA. The target mRNA has the following sequence of two bases: 5′-UG-3′. The construction and production of hammerhead ribozymes is well known in the art and is described more fully in Haseloff and Gerlach (Nature 334:585-591 (1988)). Preferably, the ribozyme is engineered so that the cleavage recognition site is located near the 5′ end of the mRNA in order to increase efficiency and minimize the intracellular accumulation of non-functional mRNA transcripts.

Ribozymes of the invention can be composed of modified oligonucleotides (for example for improved stability, targeting, and the like). DNA constructs encoding the ribozyme can be under the control of a strong constitutive promoter, such as, for example, pol III or pol II promoter, so that a transfected cell will produce sufficient quantities of the ribozyme to destroy endogenous target mRNA and inhibit translation. Since ribozymes, unlike antisense molecules, are catalytic, a lower intracellular concentration is generally required for efficiency.

The present invention also provides pharmaceutical compositions, including both therapeutic and prophylatic compositions. Compositions within the scope of this invention include all compositions wherein the therapeutic abent, antibody, fragment or derivative, antisense oligonucleotide or ribozyme is contained in an amount effective to achieve its intended purpose. While individual needs vary, determination of optimal ranges of effective amounts of each component is within the skill of the art. The effective dose is a function of a number of factors, including the specific antibody, the antisense construct, ribozyme or polypeptide of the invention, the presence of a conjugated therapeutic agent (see below), the patient and their clinical status.

Mode of administration may be by parenteral, subcutaneous, intravenous, intramuscular, intraperitoneal, transdermal, or buccal routes. Alternatively, or concurrently, administration may be orally. The dosage administered will be dependent upon the age, health, and weight of the recipient, kind of concurrent treatment, if any, frequency of treatment, and the nature of the effect desired.

Such compositions generally comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skimmed milk, glycerol, propylene, glycol, water, ethanol and the like. The composition, if desired, can also contain minor amounts of wetting or emulsifying agents, or pH buffering agents.

These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, and the like. Such compositions will contain a therapeutically effective amount of the compound, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to a human. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with anions such as those derived from hydrochloric, phosphoric, acetic, oxalic, tartaric acids, and the like, and those formed with cations such as those derived from sodium, potassium, ammonium, calcium, ferric hydroxides, isopropylamine, triethylamine, 2-ethylamino ethanol, histidine, procaine, and the like.

The compositions of the invention can be administered alone or in combination with other therapeutic agents. Therapeutic agents that can be administered in combination with the compositions of the invention, include but are not limited to chemotherapeutic agents, antibiotics, steroidal and non-steroidal anti-inflammatories, conventional immunotherapeutic agents, cytokines and/or growth factors. Combinations may be administered either concomitantly, for example, as an admixture, separately but simultaneously or concurrently; or sequentially. This includes presentations in which the combined agents are administered together as a therapeutic mixture, and also procedures in which the combined agents are administered separately but simultaneously, for example, as through separate intravenous lines into the same individual. Administration “in combination” further includes the separate administration of one of the compounds or agents given first, followed by the second.

Conventional nonspecific immunosuppressive agents, that may be administered in combination with the compositions of the invention include, but are not limited to, steroids, cyclosporine, cyclosporine analogs, cyclophosphamide methylprednisone, prednisone, azathioprine, FK-506, 15-deoxyspergualin, and other immunosuppressive agents.

In a further embodiment, the compositions of the invention are administered in combination with an antibiotic agent. Antibiotic agents that may be administered with the compositions of the invention include, but are not limited to, tetracycline, metronidazole, amoxicillin, β-lactamases, aminoglycosides, macrolides, quinolones, fluoroquinolones, cephalosporins, erythromycin, ciprofloxacin, and streptomycin.

In an additional embodiment, the compositions of the invention are administered alone or in combination with an anti-inflammatory agent. Anti-inflammatory agents that can be administered with the compositions of the invention include, but are not limited to, glucocorticoids and the nonsteroidal anti-inflammatories, aminoarylcarboxylic acid derivatives, arylacetic acid derivatives, arylbutyric acid derivatives, arylcarboxylic acids, arylpropionic acid derivatives, pyrazoles, pyrazolones, salicylic acid derivatives, thiazinecarboxamides, e-acetamidocaproic acid, S-adenosylmethionine, 3-amino-4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole, and tenidap.

In another embodiment, compositions of the invention are administered in combination with a chemotherapeutic agent. Chemotherapeutic agents that may be administered with the compositions of the invention include, but are not limited to, antibiotic derivatives (e.g., doxorubicin, bleomycin, daunorubicin, and dactinomycin); antiestrogens (e.g., tamoxifen); antimetabolites (e.g., fluorouracil, 5-FU, methotrexate, floxuridine, interferon α-2b, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cis-platin, and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chorambucil, mechlorethamine (nitrogen mustard) and thiotepa); steroids and combinations (e.g., bethamethasone sodium phosphate); and others (e.g., dicarbazine, asparaginase, mitotane, vincristine sulfate, vinblastine sulfate, and etoposide).

In an additional embodiment, the compositions of the invention are administered in combination with cytokines. Cytokines that may be administered with the compositions of the invention include, but are not limited to, IL2, IL3, IL4, IL5, IL6, IL7, IL10, IL12, IL13, IL15, anti-CD40, CD40L, IFN-gamma and TNF-α.

In additional embodiments, the compositions of the invention are administered in combination with other therapeutic or prophylactic regimens, such as, for example, radiation therapy.

The present invention is further directed to therapies which involve administering pharmaceutical compositions of the invention to an animal, preferably a mammal, and most preferably a human patient for treating one or more of the described disorders. Therapeutic compositions of the invention include, for example, therapeutic agents identified in screening assays, antibodies of the invention (including fragments, analogs and derivatives thereof as described herein), antisense oligonucleotides, ribozymes and nucleic acids encoding same. The compositions of the invention can be used to treat, inhibit, prognose, diagnose or prevent diseases, disorders or conditions associated with aberrant expression and/or activity of a polypeptide of the invention, including, but not limited to, any one or more of the diseases, disorders, or conditions such as, for example, MLL, AML, or ALL.

The treatment and/or prevention of diseases and disorders associated with aberrant expression and/or activity of a polypeptide of the invention includes, but is not limited to, alleviating symptoms associated with those diseases and disorders.

The amount of the compound of the invention which will be effective in the treatment, inhibition and prevention of a disease or disorder associated with aberrant expression and/or activity of a polypeptide of the invention can be determined by standard clinical techniques. In addition, in vitro assays may optionally be employed to help identify optimal dosage ranges. The precise dose to be employed in the formulation will also depend on the route of administration, and the seriousness of the disease or disorder. Effective doses may be extrapolated from dose-response curves derived from in vitro or animal model test systems.

For antibodies, the dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Furthermore, the dosage and frequency of administration of antibodies of the invention may be reduced by enhancing uptake and tissue penetration of the antibodies by modifications such as, for example, lipidation or addition of cell-specific tags.

The compounds or pharmaceutical compositions of the invention can be tested in vitro, and then in vivo for the desired therapeutic or prophylactic activity, prior to use in humans. For example, in vitro assays to demonstrate the therapeutic or prophylactic utility of a compound or pharmaceutical composition include, the effect of a compound on a cell line or a patient tissue sample. The effect of the compound or composition on the cell line and/or tissue sample can be determined utilizing techniques known to those of skill in the art including, but not limited to, rosette formation assays and cell lysis assays. In accordance with the invention, in vitro assays which can be used to determine whether administration of a specific compound is indicated, include in vitro cell culture assays in which a patient tissue sample is grown in culture, and exposed to or otherwise administered a compound, and the effect of such compound upon the tissue sample is observed. One example of a cell line or cell population is the MLL-AF9 cell line, wherein MLL-AF9 is expressed in myeloid progenitor or hematopoietic stem cells and the cells are immediately injected into recipient mice. Upon leukemia development, IL-7⁻ lin− Sca-1⁻ c-Kit⁺ CD34⁺ FcγRII/III⁺ (L-GMP), are isolated. Cell lines are subsequently generated by passage of the cells in culture in the presence of interleukin-3.

The invention provides methods of treatment, inhibition and prophylaxis by administration to a subject of an effective amount of a compound or pharmaceutical composition of the invention. In one aspect, the compound is substantially purified such that the compound is substantially free from substances that limit its effect or produce undesired side-effects. The subject is preferably an animal, including but not limited to animals such as cows, pigs, horses, chickens, cats, dogs, etc., and is preferably a mammal, and most preferably human.

Various delivery systems are known and can be used to administer a composition of the invention, for example, encapsulation in liposomes, microparticles, microcapsules, recombinant cells capable of expressing the compound, receptor-mediated endocytosis (see, e.g., Wu and Wu, J. Biol. Chem. 262:4429-4432 (1987)), construction of a nucleic acid as part of a retroviral or other vector, and the like as will be known by one of skill in the art.

Methods of introduction include, but are not limited to, intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, for example, by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, for example, in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the invention, care must be taken to use materials to which the protein does not absorb.

In another embodiment, the compound or composition can be delivered in a vesicle, such as a liposome (Langer, Science 249:1527-1533 (1990)).

In yet another embodiment, the compound or composition can be delivered in a controlled release system. Furthermore, a controlled release system can be placed in proximity of the therapeutic target, thus requiring only a fraction of the systemic dose (see, e.g., Goodson, in Medical Applications of Controlled Release, supra, vol. 2, pp. 115-138 (1984)). In a further embodiment, a pump may be used. In another embodiment, polymeric materials can be used.

In a particular embodiment where the compound of the invention is a nucleic acid encoding a protein, the nucleic acid can be administered in vivo to promote expression of its mRNA and encoded protein, by constructing it as part of an appropriate nucleic acid expression vector and administering, for example, by use of a retroviral vector, or by direct injection, or by use of microparticle bombardment for example, a gene gun, or coating with lipids or cell-surface receptors or transfecting agents, or by administering it in linkage to a homeobox-like peptide which is known to enter the nucleus (see e.g., Joliot et al., Proc. Natl. Acad. Sci. USA 88:1864-1868 (1991)). Alternatively, a nucleic acid can be introduced intracellularly and incorporated within host cell DNA for expression, by homologous recombination.

Therapeutic nucleic acid molecules (e.g., enzymatic nucleic acid molecules and antisense nucleic acid molecules) delivered exogenously are optimally stable within cells until translation of the target RNA has been inhibited long enough to reduce the levels of the undesirable protein. This period of time varies between hours to days depending upon the disease state. These nucleic acid molecules should be resistant to nucleases in order to function as effective intracellular therapeutic agents. Improvements in the chemical synthesis of nucleic acid molecules described in the instant invention and in the art have expanded the ability to modify nucleic acid molecules by introducing nucleotide modifications to enhance their nuclease stability as described above.

In still another embodiment, the present invention relates to the identification of individual genes that can be targeted for therapy. For example, the below-demonstration that shRNA mediated inhibition of Mef2c leads to decreased LSC proliferation and survival provides support for this idea. Furthermore, individual genes were tested as potential therapeutic targets in human leukemia. Expression of Mef2c, EphA7, RunxZ, Nln, Tcf4, Meis1, HoxA9, Galgt, Fdx1, HoxA10, and HoxA5 was suppressed in MLL-rearranged human leukemia cell lines. This suppression inhibited leukemia cell proliferation and survival, thus making these genes, for example, potential therapeutic targets. The self-renewal program described herein is likely to be at least partially active in all cancer stem cell; therefore, the therapeutics described herein for leukemia may be of benefit in other cancers.

In one embodiment, nucleic acid catalysts having chemical modifications that maintain or enhance enzymatic activity are provided. Such nucleic acids are also generally more resistant to nucleases than unmodified nucleic acid. Thus, in a cell and/or in vivo the activity of the nucleic acid can not be significantly lowered. As exemplified herein such enzymatic nucleic acids are useful in a cell and/or in vivo even if activity over all is reduced about 10 fold (Burgin et al., 1996, Biochemistry, 35, 14090). Such enzymatic nucleic acids herein are said to “maintain” the enzymatic activity of an all RNA ribozyme or all DNA DNAzyme.

In another aspect of the invention, vectors, preferably expression vectors, contain nucleic acids encoding one or more siNAs, for example miRNAs. As used herein, the term “vector” refers to a nucleic acid molecule capable of transporting another nucleic acid to which it has been linked. One type of a vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g. bacterial vectors having a bacterial origin of replication and episomal mammalian vectors. Other vectors (e.g. non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form plasmids. In the present specification, “plasmid” and “vector” can be used interchangeably as the plasmid is the most commonly used from of vector. However, the invention is intended to include such other forms of expression vectors, such as viral vectors (e.g. replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g. in an in vitro transcription/translation system or in a host cell when the vector is introduced into the host cell). The term “regulatory sequence” is intended to included promoters, enhancers, and other expression control elements (e.g. polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g. tissue-specific regulatory sequences). It will be appreciated by those skilled in the art that the design of the expression vector can depend on such factors as the choice of the host cell to be transformed, the level of expression of protein or RNA desired, etc. The expression vectors of the invention can be introduced into host cells to thereby produce siNAs, RNAs, proteins or peptides, including fusion proteins or peptides.

In another embodiment, a nucleic acid of the invention is expressed in mammalian cells using a mammalian expression vector. The recombinant mammalian expression vector may be capable of directing expression of the nucleic acid preferentially in a particular cell type (e.g. tissue-specific regulatory elements are used to express the nucleic acid). Tissue specific regulatory elements are known in the art. Non-limiting examples of suitable tissue-specific promoters include the albumin promoter, lymphoid-specific promoters, neuron specific promoters, pancreas specific promoters, and mammary gland specific promoters. Developmentally-regulated promoters are also encompassed, for example the murine hox promoters and the α-fetoprotein promoter.

In another aspect the nucleic acid molecules comprise a 5′ and/or a 3′-cap structure. By “cap structure” is meant chemical modifications, which have been incorporated at either terminus of the oligonucleotide (see for example Wincott et al, WO 97/26270, incorporated by reference herein). These terminal modifications protect the nucleic acid molecule from exonuclease degradation, and can help in delivery and/or localization within a cell. The cap can be present at the 5′-terminus (5′-cap) or at the 3′-terminus (3′-cap) or can be present on both terminus. In non-limiting examples, the 5′-cap includes inverted abasic residue (moiety), 4′,5′-methylene nucleotide; 1-(β-D-erythrofuranosyl) nucleotide, 4′-thio nucleotide, carbocyclic nucleotide; 1,5-anhydrohexitol nucleotide; L-nucleotides; α-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270, incorporated by reference herein).

In another embodiment the 3′-cap includes, for example 4′,5′-methylene nucleotide; 1-(β-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; α-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

The administration of the herein described nucleic acid molecules to a patient can be intravenous, intraarterial, intraperitoneal, intramuscular, subcutaneous, intrapleural, intrathecal, by perfusion through a regional catheter, or by direct intralesional injection. When administering these nucleic acid molecules by injection, the administration may be by continuous infusion, or by single or multiple boluses. The dosage of the administered nucleic acid molecule will vary depending upon such factors as the patient's age, weight, sex, general medical condition, and previous medical history. Typically, it is desirable to provide the recipient with a dosage of the molecule which is in the range of from about 1 pg/kg to 10 mg/kg (amount of agent/body weight of patient), although a lower or higher dosage may also be administered.

A composition is said to be a “pharmaceutically acceptable carrier” if its administration can be tolerated by a recipient patient. Sterile phosphate-buffered saline is one example of a pharmaceutically acceptable carrier. Other suitable carriers are well-known in the art. See, for example, REMINGTON'S PHARMACEUTICAL SCIENCES, 18^{th} Ed. (1990).

For purposes of immunotherapy, an immunoconjugate and a pharmaceutically acceptable carrier are administered to a patient in a therapeutically effective amount. A combination of an immunoconjugate and a pharmaceutically acceptable carrier is said to be administered in a “therapeutically effective amount” if the amount administered is physiologically significant. An agent is physiologically significant if its presence results in a detectable change in the physiology of a recipient patient.

Additional pharmaceutical methods may be employed to control the duration of action of an immunoconjugate in a therapeutic application. Control release preparations can be prepared through the use of polymers to complex or adsorb an immunoconjugate. For example, biocompatible polymers include matrices of poly(ethylene-co-vinyl acetate) and matrices of a polyanhydride copolymer of a stearic acid dimer and sebacic acid. Sherwood et al., Bio/Technology 10:1446-1449 (1992). The rate of release of nucleic acid molecule from such a matrix depends upon the molecular weight of the molecule, the amount of molecule within the matrix, and the size of dispersed particles. Saltzman et al., Biophysical. J. 55:163-171 (1989); and Sherwood et al., Bio/Technology 10:1446-1449 (1992). Other solid dosage forms are described in REMINGTON'S PHARMACEUTICAL SCIENCES, 18th Ed. (1990).

Having now generally described the invention, the same will be more readily understood through reference to the following Examples which are provided by way of illustration, and are not intended to be limiting of the present invention.

### Example 1

### Acute Myelogenous Leukemia Induction

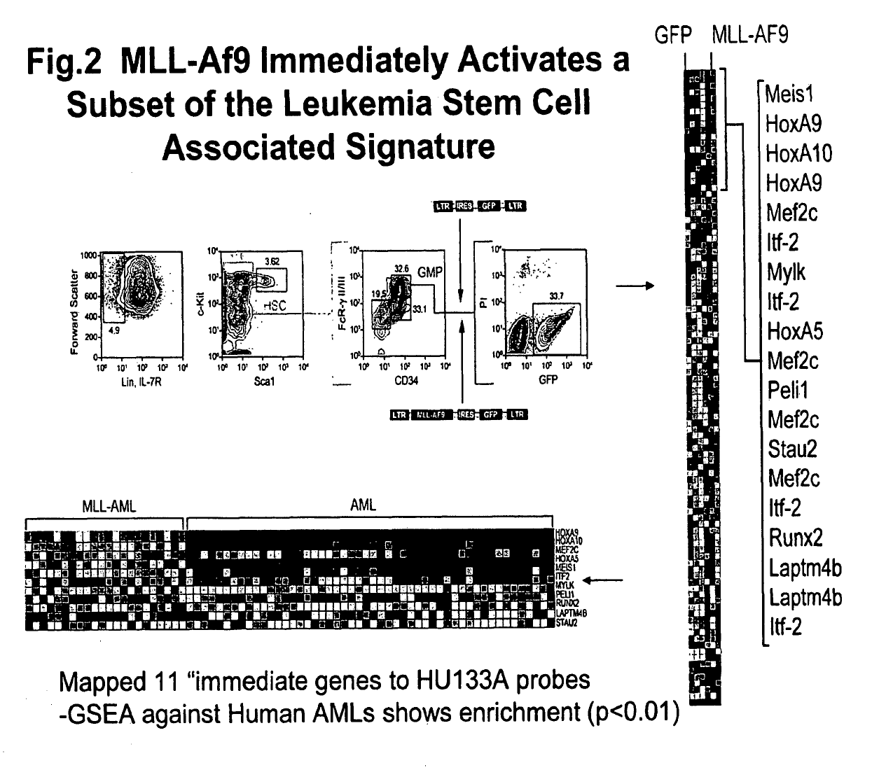

Fusion proteins encoded by translocations involving the mixed lineage leukemia (MLL) gene have been reported to be capable of imparting leukemia stem cell properties on committed progenitors. 9,11. A MLL-AF9 fusion protein was expressed in highly purified IL-7R⁻ Lin⁻ Sca-1⁻ c-Kit⁺ CD34⁺ FcγRII/III^{hi} granulocyte-macrophage progenitors (GMP) 12 (FIG. 2). The purity and identity of the sorted GMP population was verified both by assays of colony forming activity, and by comparison of gene expression profiles with previously published reports (data not shown). 13, 14. Transduction of GMP with a retrovirus encoding MLL-AF9 (MLL-AF9-GFP), and subsequent cultivation in methylcellulose media in the absence of stroma demonstrated enhanced colony formation in a serial replating assay in cultures initiated by MLL-AF9, but not a control retrovirus that encodes only GFP (MCV-GFP). 9,15. MLL-AF9 transduced GMP also induced AML in vivo. Sublethally irradiated mice were transplanted with a limiting dilution of MLL-AF9-GFP transduced GMPs or 1×10⁴ MSCV-GFP transduced GMP (Table 1). Mice injected with MSCV-GFP demonstrated a transient population of GFP+ peripheral blood leukocytes within 2-3 weeks after transplantation, but there was no sustained hematopoiesis from GFP+ cells. Also, non of the animals transplanted with MSCV-GFP transduced GMP (n=7) developed AML (Table 1). In contrast, within 80 days all animals transplanted with 10,000 or 5,000 GMPs transduced with MSCV-MLL-AF9 developed an oligoclonal Mac-1+, GR-1+, CD3−, B220− AML similar to other MLL-fusion leukemia models. 9, 16-19. Only 1 out of 5 mice transplanted with 1000 cells developed AML. None of the mice transplanted with either 100 (n=5) or 20 (n=5) cells developed AML (Table 1). Next, a determination was made as to whether the AMLs initiated from GMP were transferable to secondary recipients. Syngeneic recipient mice were sublethally irradiated and injected with anywhere from 20 to 1×10⁴ bone marrow cells from leukemic mice. All of the secondary recipient mice that received 500 or greater leukemic cells died of AML. Seven of 14 mice that received 100 cells and none of the mice that received 20 cells succumbed to AML (Table 1). Therefore, the calculated frequency of leukemia initiating cells in bone marrow is approximately 1:150. Thus, MLL-AF9 induces self-renewal and leukemia from prospectively purified GMP.

### Example 2

### Leukemia Stem Cells are Present in GMP-Like Leukemic Cell Populations

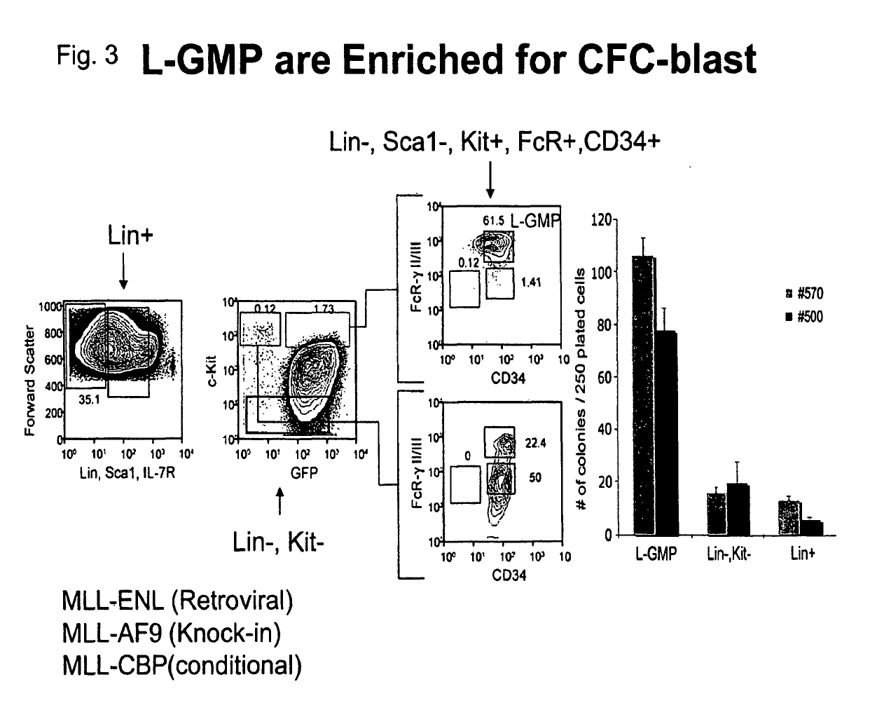

Retroviral or knock-in models of MLL-fusion induced leukemias demonstrate the presence of GMP-like leukemic cells (Leukemic-GMP) (FIG. 3). 9,18. Therefore, Leukemic-GMP (L-GMP) might contain LSC. An initial assessment of L-GMP was undertaken to determine whether these cells were enriched for LSC. We sorted Lin⁺, IL-7R⁻ Lin⁻ Sca-1⁻ c-Kit⁻, or IL-7R⁻ lin⁻ Sca-1⁻ c-Kit⁺ CD34⁺ FcγRII/III⁺ (L-GMP) cells from mice that developed AML initiated from MLL-AF9 transduced GMP (FIG. 3), and cultured them in methylcellulose containing interleukin-3 (IL3), stem cell factor (SCF), and interleukin-6 (IL6). L-GMP cells possessed higher colony forming activity than the other populations (FIG. 3), further directing efforts toward characterization of this population.

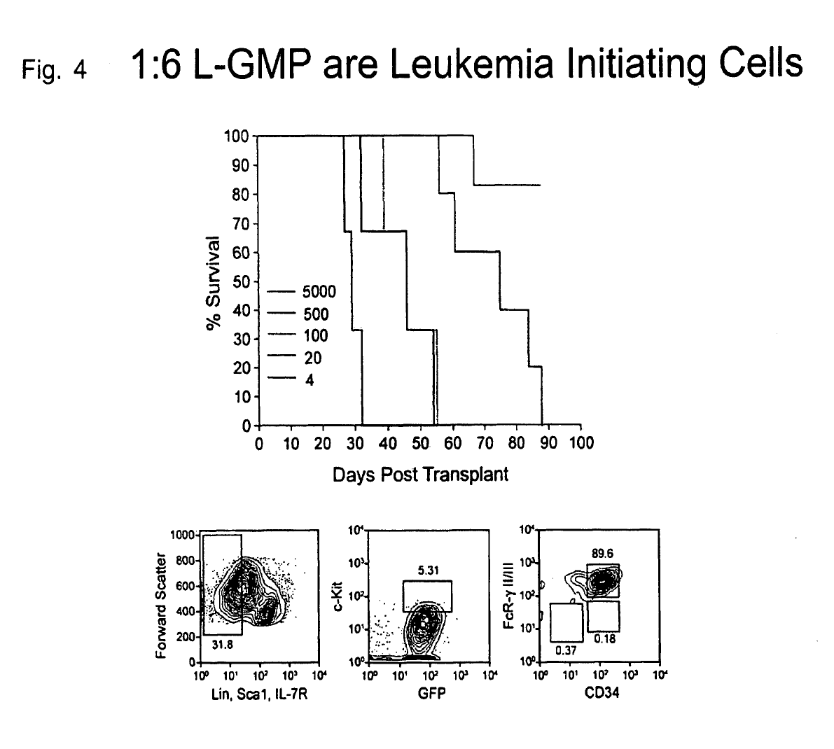

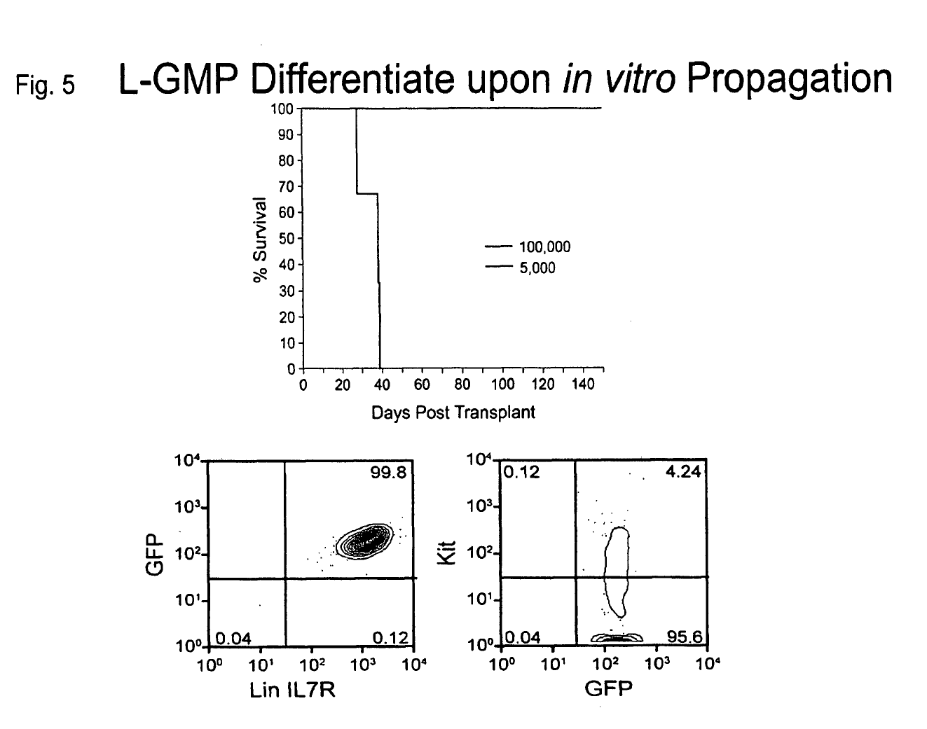

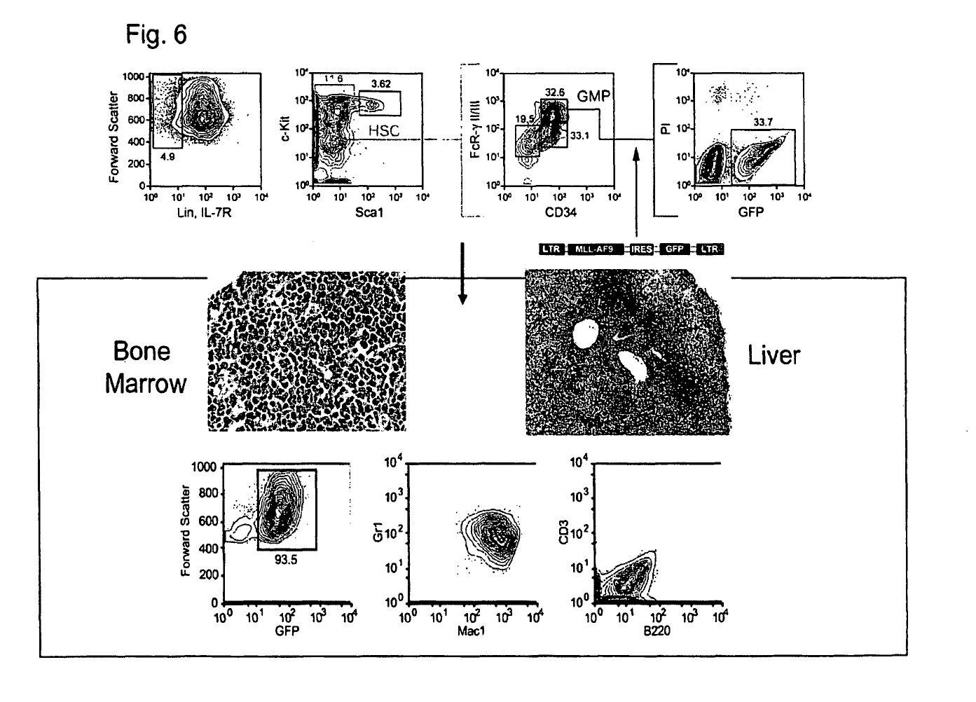

Sublethally irradiated syngeneic recipients were transplanted with 5,000 (n=11), 500 (n=7), 100 (n=11), 20 (n=22), or 4 (n=6) sorted L-GMP and assessed for leukemia. Within 80+/−7 days, each of the secondary recipients transplanted with from about 20 to 5×10³ L-GMP developed AML that was phenotypically identical to the primary disease (FIGS. 4, 5, and 6). One of 6 mice injected with 4 L-GMP died of AML (FIG. 4). The remaining 5 mice injected with 4 L-GMP had no GFP positive bone marrow, spleen, or peripheral blood cells when the experiment was terminated at 100 days. Thus, the L-GMP population contains leukemia initiating cells with a calculated frequency of approximately 1:6 (/-2). In addition, a high frequency of leukemia initiating cells in the L-GMP populations was confirmed by plating L-GMP in methylcellulose. Single colonies were isolated after 5 days in culture and injected into sublethally irradiated syngeneic recipients. Fifteen out of 30 mice receiving cells from single colonies died of AML within 70 days. The remaining mice did not have GFP positive bone marrow cells when the experiment was terminated at 100 days (data not shown). To further characterize the stem cell like properties of L-GMP, we assessed their potential to generate differentiated progeny. L-GMP were easily propagated in liquid culture containing IL-3, a well-described characteristic of murine MLL-fusion induced leukemias. 16. However, after conversion to liquid culture the majority of the cells (AKLG cells) demonstrated a more differentiated, lineage+, Kit− immunophenotype (FIG. 5). This more differentiated phenotype was seen in conjunction with an increase in the number of cells needed to initiate leukemia in secondary recipients to >5,000 (FIG. 5). These data demonstrate that leukemia cells with an immunophenotype similar to normal GMP are highly enriched for leukemia stem cells that can both initiate leukemia in secondary recipient mice and produce more differentiated progeny incapable of transferring the disease.

### Example 3

### Gene Expression Alterations During the Transition from Committed Progenitor to Leukemia Stem Cell

Having prospectively purified a population highly enriched for LSC, gene expression analysis was used as a tool to assess cellular identity and to characterize gene expression changes that occur during the transition from committed progenitor to leukemia stem cell. Given that isolation of L-GMP is dependent upon the expression of a limited number of immunophenotypeic markers, the gene expression profile of L-GMP was assessed to determine whether it remained similar to the normal GMP from which they arose; or if global cellular reprogramming had occurred during the transformation.

Morphologically, the L-GMP and normal GMP were uniformly small cells with indented nuclei consistent with some degree of myelomoncytic differentiation. In order to compare gene expression profiles between normal progenitors and L-GMP, we isolated the IL7R⁻ Lin⁻ Sca-1⁺ c-kit⁺ HSC-enriched population, IL-7R⁻ Lin⁻ Sca-1⁻ c-Kit⁺ CD34⁺ FcγRII/III^{lo} common myeloid progenitors (CMP), IL-7R⁻ Lin⁻ Sca-1⁻ c-Kit⁺ CD34⁻ FcγRII/III⁻ megakaryocyte erythroid progenitors (MEP), and GMP from 6-8 week old C57BL/6 mice. We also isolated L-GMP from mice that were approximately 60 days post transplant and had clinically evident leukemia. RNA was isolated from each of the populations, amplified and hybridized to Affymetrix murine 430A 2.0 microarrays. Unsupervised analysis using hierarchical clustering demonstrated that the L-GMP have a stable gene expression program that is different from any of the normal populations (FIG. 7). However, the L-GMP population has a gene expression signature that is globally more similar to the GMP from which they arose than any other normal population (FIG. 7). K-means clustering identified a large group of genes that demonstrate high-level expression in normal GMP as compared to other normal progenitors and HSCs, and expression that is further elevated in the L-GMP population (FIG. 7, signature 1a). The inverse of this profile was similarly prominent (FIG. 7, signature 1b). These data show the L-GMP population possesses a global cellular program that is distinct from normal cells, but most similar to the GMP from which they arose.

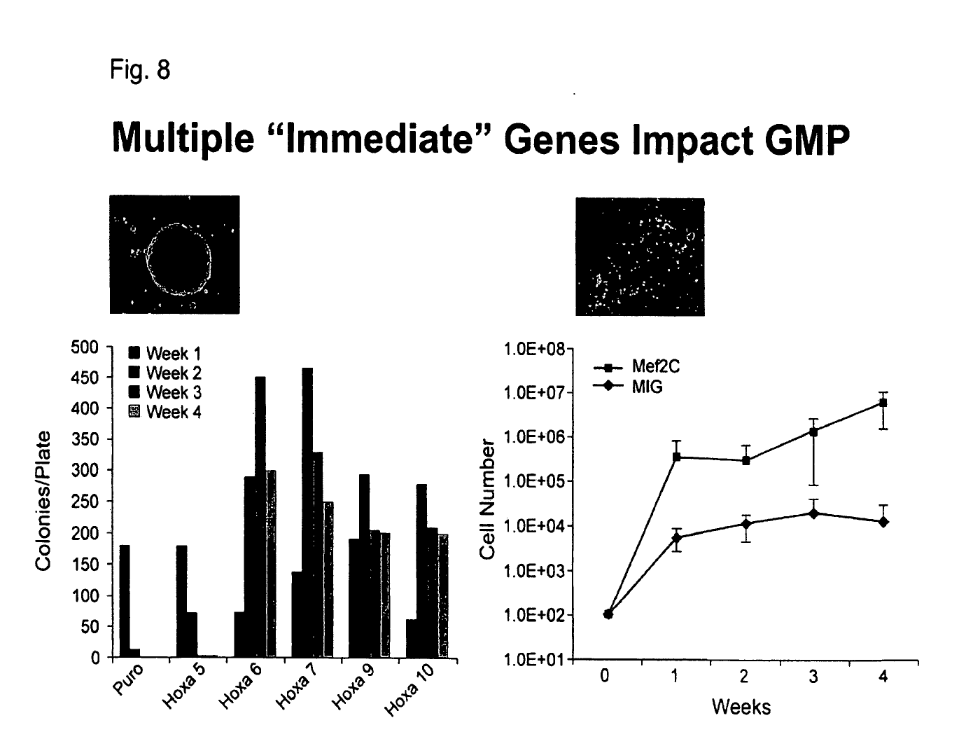

A group of genes was highly expressed in the HSC-population, showed decreased expression in committed progenitors, but have re-activated high-level expression in the L-GMP population (signature 3a in FIG. 7), and the inverse (signature 3b in FIG. 7). Supervised analysis identified approximately 420 probe sets for 363 genes that were more highly expressed in populations with self-renewal potential (HSC and L-GMP) than in progenitors with no self-renewal potential (FIG. 7), and approximately 300 probe sets for 262 genes that are more highly expressed in committed progenitors than either HSC or LSC. Of interest, the 363 genes that are more highly expressed in HSC and LSC (a self-renewal associated signature) represents only a subset of the approximately 1137 genes normally highly expressed in HSC compared to committed progenitors (FIG. 7). To further validate our HSC signature and the ˜363-gene self-renewal associated signature as genes enriched in stem cells we compared our data to a previously published gene expression analysis that assessed gene expression in long-term hematopoietic stem cells (LT-HSC). 20. Both the self-renewal associated signature and the remainder of our HSC signature showed significant overlap with the previous LT-ITSC data. Next, we assessed if the self-renewal associated signature is lost upon propagation of the L-GMP in liquid culture under conditions that support robust in vitro growth, but leads to a decrease in the LSC frequency (FIG. 5 c,d). When L-GMP differentiate into a non self-renewing population (AKLG cells) the self-renewal associated signature largely reverts back to the levels found in normal GMP, even though the cells remain immortal (FIG. 8d). Finally, the presence of a number of transcription factors that are important in normal hematopoietic development, and in leukemogenesis mediated by MLL-fusions including multiple Hox genes (15, 21-24), supports the relevance of this signature. These data show that acquisition of self-renewal properties in committed progenitors is associated with activation of a program highly expressed in normal hematopoietic stem cells.

### Example 4

### Assessing LSC/L-GMP Population Homogeneity

Given that the calculated frequency of LSC in L-GMP is 1:6, we wanted to further assess the homogeneity of this population. First, a search was conducted for genes in the self-renewal associated signature encoding cell surface molecules that might be assessed by flow cytometry. One such gene, CD48 is a member of the Slam family of cell surface molecules recently shown to be part of a Slam-family signature in normal hematopoietic stem cells²⁵. Gene expression data demonstrated CD48 is more highly expressed in GMP than in either HSC or L-GMP (signature 3b in FIG. 7). Assessment of CD48 expression on the surface of GMP and L-GMP confirmed decreased expression in L-GMP, and also demonstrated homogeneous expression in the L-GMP population (FIG. 7). While this homogenous staining pattern of CD48 in L-GMP suggests genes in the self-renewal associated signature are not merely a “visible” portion of the full HSC signature expressed in 1:6 L-GMPs, we further assessed this using a computational approach. If the self-renewal associated signature is only the visible portion of the full HSC signature, then the genes in this signature should be the most differentially expressed genes in a comparison between HSC and normal committed progenitors. However, the genes in the self-renewal associated signature are distributed randomly throughout the HSC signature. These data show L-GMP is a homogeneous population, and demonstrate the potential utility of the self-renewal associated signature for the identification of markers that might be used to further characterize LSC.

### Example 5

### Expression of Self-Renewal Associated Gene Expression Program in Human MLL-Rearranged AML

To assess the relevance of the murine signature in human AML, we analyzed the expression of this self-renewal associated gene expression program in human MLL-rearranged AML as compared to AML with other chromosomal translocations. Gene set enrichment analysis (GSEA)²⁶ demonstrated significant overlap between the murine self-renewal associated signature and the human MLL-AML signature with approximately 91 genes more highly expressed in MLL-rearranged AMI- (p=0.016). Thus, a portion of the self-renewal associated program is highly expressed in MLL-rearranged AML supporting the relevance of this signature in human AML. Future studies will determine if the genes that are similarly expressed in all AML samples are part of a universal AML self-renewal signature.

Having identified a self-renewal associated signature present in leukemia stem cells, we wished to determine if it is fully activated immediately after MLL-AF9 expression or if there is a hierarchy of gene expression changes that might help us focus on individual genes. We transduced 5×10⁴-1×10⁵ GMP with either MLL-AF9-GFP or MSCV-GFP retroviruses. Forty hours later 2×10⁴-5×10⁴ GFP⁺ PI⁻ cells were re-sorted from both samples, RNA was isolated and hybridized to microarrays. We assessed changes in expression of the genes in the self-renewal associated signature and found that a small subset of these genes showed increased expression in the MLL-AF9-GFP transduced GMP. These “immediate” genes included genes previously implicated in MLL-rearranged leukemias HoxA5, HoxA9, HoxA10, Meis1, and new genes not known to have an association with MLL-rearrangement such as Mef2c, Runx.2 and the wnt pathway associated gene Ift-2. Thus, a subset of genes are immediately activated by MLL-AF9 expression suggesting a hierarchy of gene expression where the total self-renewal associated signature is a summation of multiple gene expression programs.

In an attempt to further validate the findings in our murine system, we determined if there was significant enrichment of the 11-gene “immediate” signature in human MLL-rearranged AMLs as compared to AMLs with other chromosomal translocations. GSEA demonstrated significant enrichment (p˜0.004) of the immediate signature in human MLL-rearranged AMLs. Remarkably, 6 of 11 genes in the signature are more highly expressed in the MLL-AML samples than in AMLs with other chromosomal translocations. These data provide further support for the relevance of findings in our murine model to human leukemia, and point to specific genes as potentially relevant for leukemia stem cell development.

### Example 6

### Temporal Self-Renewal Signature Gene Expression

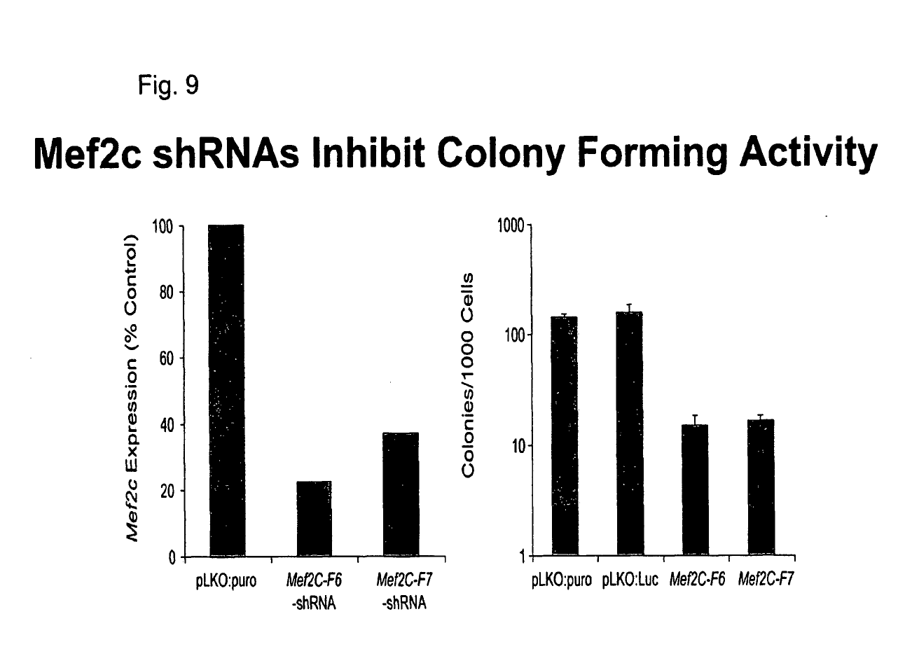

Next we focused our attention on genes whose expression increases immediately after MLL-AF9 expression in GMP, are part of the self-renewal associated signature, and are more highly expressed in human MLL-rearranged AML. Given the prominent role HoxA cluster genes play in MLL-rearranged and other leukemias²⁷, and the fact that MLL may regulate chromatin structure near the 5′ HoxA cluster^{28,29} we were interested to know if any of the 5′ HoxA cluster genes could impart serial replating activity on GMP. We cloned cDNAs encoding HoxA5-A10 and expressed them in isolated GMP. Remarkably, HoxA6, HoxA7, HoxA9 or HoxA10, but not HoxA5, were capable of inducing serial replating activity (FIG. 8). Also, the tight correlation between MLL-AF9 and Mef2c expression prompted a similar but more detailed assessment. Expression of Mef2c, a member of the myocyte enhancer factor 2 family of transcription factors, in GMP consistently produced an approximate 5-10 fold increase in the number of cells obtained from GMP cultures, but did not induce serial replating activity (FIG. 8). This observation is in keeping with a recent retroviral mutagenesis study that identified Mef2c as a cooperating oncogene in leukemogenesis that is unable to induce leukemia when expressed alone³⁰. Next, we expressed two Mef2c-directed shRNA and a control shRNA in L-GMP. The two Mef2c-directed shRNAs were encoded by (1) Mef2c-F6-GCCTCAGTGATACAGTATAAA (SEQ ID NO:1) and (2) Mef2c-F7-CCATCAGTGAATCAAAGGATA (SEQ ID NO:2). The Mef2c-shRNAs not only suppressed Mef2c expression, but also inhibited L-GMP colony forming activity (FIG. 9). Finally, we isolated individual colonies grown from L-GMP transduced with a control virus or a virus expressing the Mef2c-F6 shRNA, and injected them into sublethally irradiated syngeneic recipient mice. As noted previously, 56% ( 17/30) of the mice injected with control L-GMP colonies developed a lethal disease consistent with AML. However, only 20% ( 4/20) of mice injected with Mef2c-F6 transduced L-GMP developed AML. These data support a role for Hox genes and Mef2c in leukemia stem cell survival/proliferation. Furthermore, the identification of genes that influence the phenotype of L-GMP provides support for the notion that genes in the self-renewal associated signature play a critical role in leukemia stem cell development.

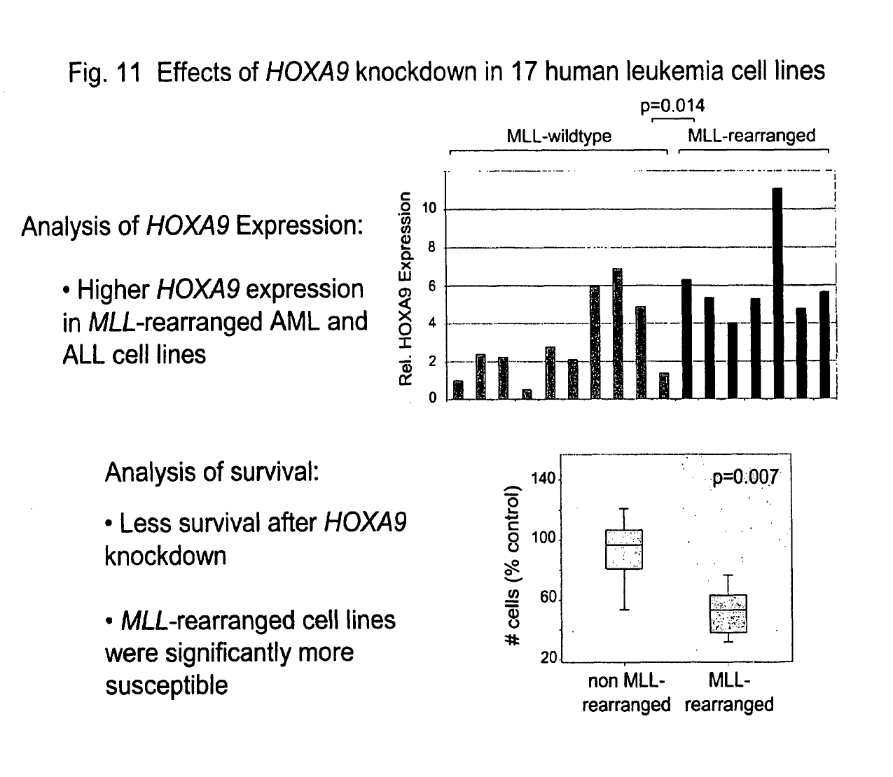

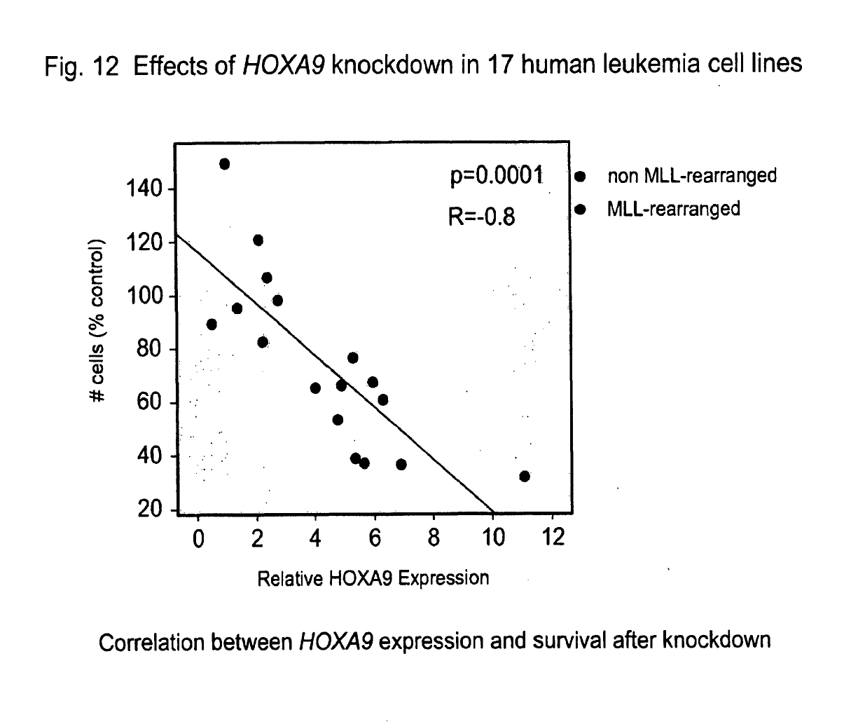

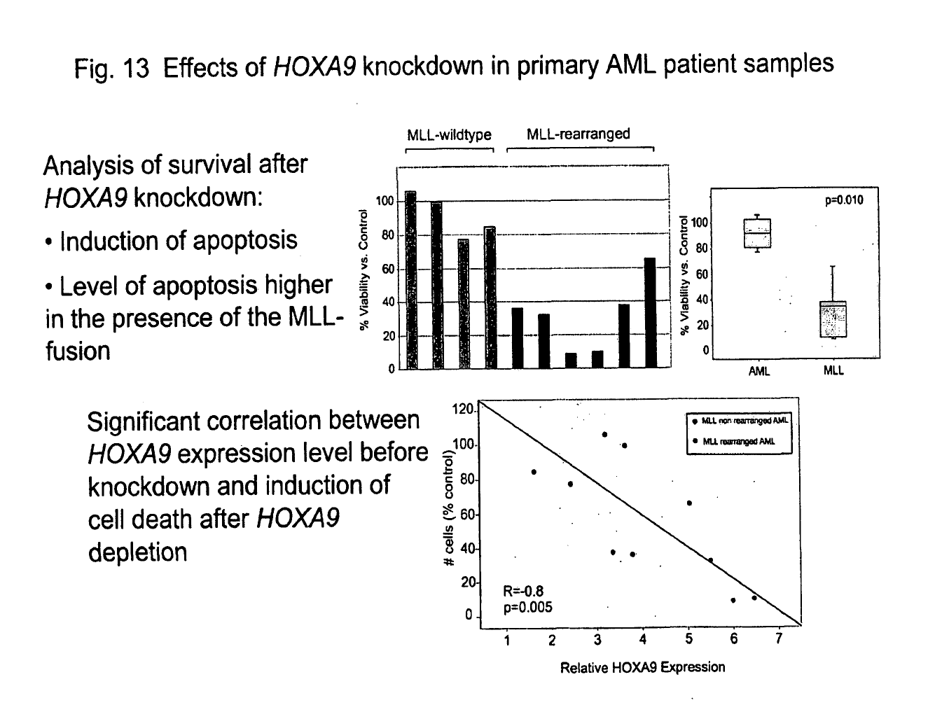

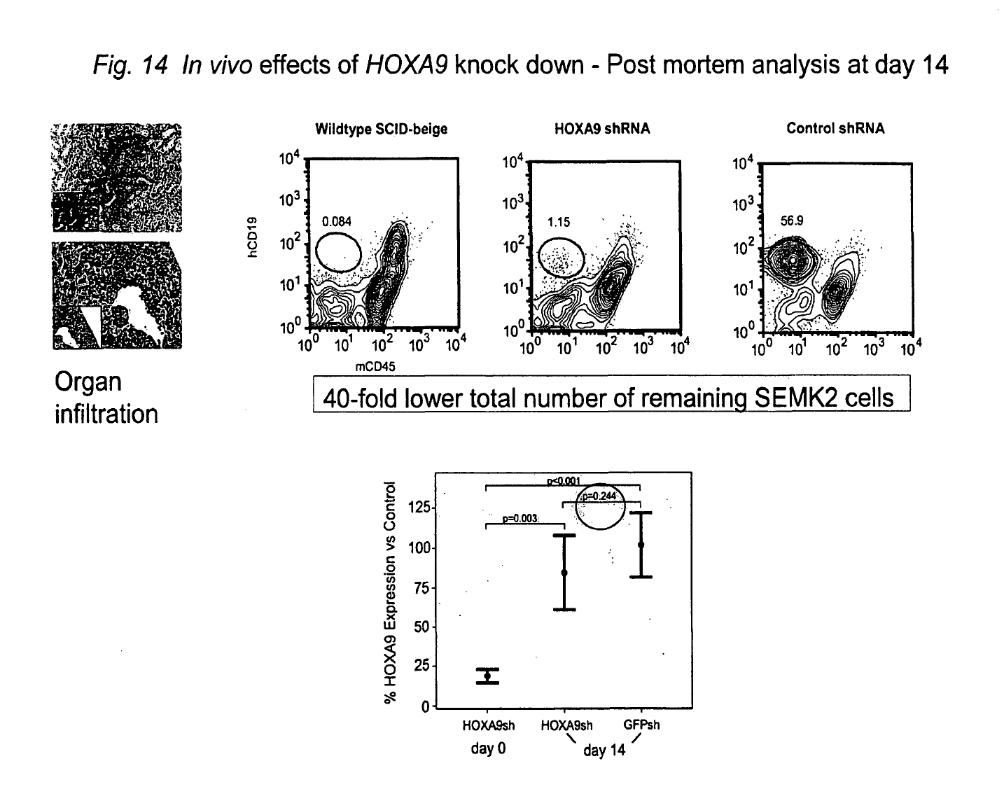

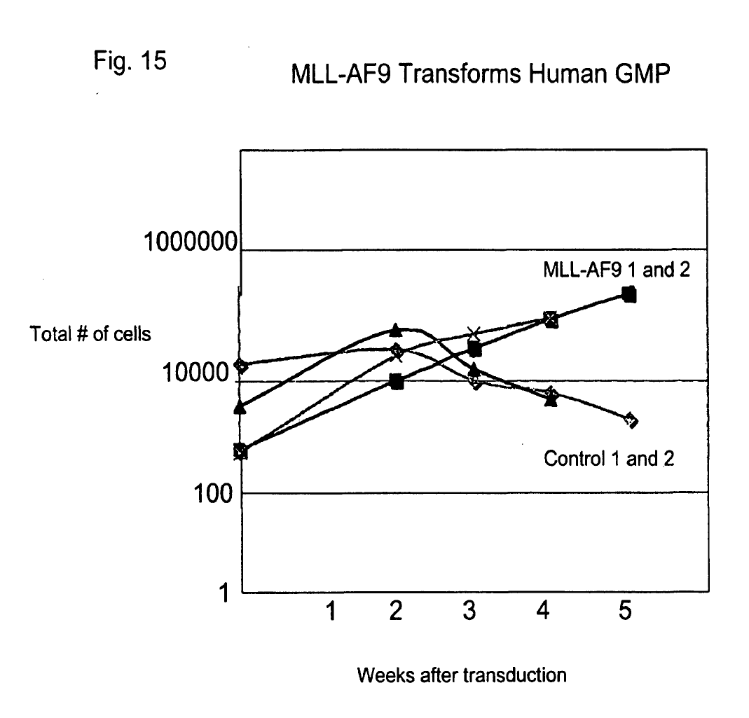

HoxA9 is highly expressed in the vast majority of MLL-ALL and MLL-AML, and is expressed in many AMLs with normal cytogenetics. Translocations involving NUP98 and HOXA9 are found these AMLs, and overexpression in mice induces AML. FIGS. 11-15 illustrate the effects of HOXA9 knockdown in 17 human leukemia cell lines (FIGS. 11 and 12), primary AML patient samples (FIG. 13), and in vivo (FIG. 14). In the present study, SEMK2 ALL cells were transduced with HOXA9 shRNA or control shRNA. The cells were subsequently transplanted into SCID/beige mice and the mice were then subjected to in vivo bioluminescence imaging. Equal numbers of transduced cells were transplanted into each mouse. Results show a significantly reduced leukemia burden and prolonged survival of those mice subjected to HOXA9 shRNA treatment. With regard to FIG. 14 (the in vivo effects of HOXA9 knockdown—Post mortem analysis at day 14), the results show an 80% knockdown at day 0 and equal expression at day 14. SEMK2 cells that escaped HOXA9 knockdown show survival advantage in vivo.

### Example 7

The self-renewal associated signature is a subset of the HSC signature-comparison with a previously published HSC signature. Due to the presence of MPP in our HSC population, it was possible that some portion of our HSC-associated signature is actually part of the MPP signature, and thus the true HSC signature might be identified only when we add the L-GMP to the analysis. To assess this, we first identified the genes highly expressed in our HSC population compared to other normal progenitors (approximately 1300 genes), and ranked them based on correlation with the profile of high expression in HSC and L-GMP and low level expression in progenitors. Next, we divided our HSC signature into those genes that are also highly expressed in L-GMP and those that are not, and looked for enrichment (with GSEA) of these genes in a previously published dataset that compares highly purified long-term-HSC (LT-HSC) to total bone marrow¹⁹. The genes highly expressed in HSC and L-GMP are enriched in the LT-HSC signature, as is the remainder of the HSC signature. Therefore the self-renewal associated signature we have identified is a subset of a larger HSC signature.

### Example 8

The self-renewal associated signature is not merely a prominent portion of the HSC signature. As L-GMP contain leukemia stem cells with a calculated frequency of 1:6, it remained possible that the self-renewal associated signature was merely a prominent portion of the complete HSC signature expressed in only 1:6 L-GMP. To address this, we first ranked genes based on correlation with high-level expression in HSC compared to normal progenitors (leaving out L-GMP expression in determination of ranking) and assessed where genes in the self-renewal signature fell in a ranked list. Genes in the self-renewal associated signature are distributed throughout the list without obvious clustering toward the top. Members of the 420-gene self-renewal associated signature were identified. Next, we performed a GSEA using the 420-gene self-renewal associated as a gene set against the HSC signature. As expected, there was no enrichment of this signature toward the top of the list (p=0.6). Thus, it is not the case that the self-renewal signature is a prominent portion of the full HSC signature that is identifiable above the background “GMP-like” signature.

### Example 9

Potential therapeutic targets in the LSC gene expression profile. An MLL-rearranged leukemia cell line and a control cell line were treated with shRNA constructs that suppress expression of select genes expressed in LSC. Cell proliferation/survival was quantified using a colorimetric assay (MTT). The relative number of surviving cells were calculated. The data shows decreased survival for the MLL-rearranged cells incubated with the shRNA constructs.

### Example 10

The self-renewal associated signature is found in human MLL-rearranged AML and is activated as a hierarchy of gene expression. GSEA was performed to assess the murine self-renewal associated signature in human MLL-AML as compared to other AML. The 363 genes for the murine signature were mapped to the appropriate human probe sets on the HU133A microarray and used as a gene set. The genes on the HU133A microarrays were ranked based on their correlation with the MLL-AML vs. AML distinction. GSEA demonstrated significant overlap (p=0.016) with 91/363 genes enriched in the human MLL-AML signature. This general approach can be used, for example, to determine if the “self-renewal associated signature” is present in a tissue sample in order to provide diagnostic or prognostic information. For example, the gene expression profile from a bone marrow sample taken from a patient believed to have leukemia could be compared to a gene expression profile from control RNA isolated from normal human progenitor cells. Using the algorithm described above (GSEA) one could determine if there is a statistically significant enrichment of the self-renewal signature in the patient sample. A significant enrichment (p<0.05) would identify the signature as present in the sample and thus provide diagnostic or prognostic information about the sample of interest. The human data set was previously published³² and includes all samples that have a defined translocation.

We transduced 5×10⁴-1×10⁵ GMP with either MLL-AF9-GFP or MSCV-GFP retroviruses, and isolated RNA 40 hours later sorted from 2×10⁴-5×10⁴ GFP⁺ PI⁻ cells. Two rounds of in vitro transcription was performed and RNA hybridized to Affymetrix 430A 2.0 microarrays. This experiment was repeated 3 times. The 420 probe sets from the self-renewal associated signature were ranked according to the distinction of high-level expression in MLL-AF9-GFP transduced cells compared to MSCV-GFP transduced cells using the t-test statistic. The top 100 probe sets in this ranking are shown. 11 genes had a t-test score >2.0 and are labeled to the right. Genes with a score <2.0 are shaded.

The 11-gene set was assessed in MLL-rearranged human AMLs as compared to human AMLs with other translocations³². GSEA demonstrated a significant overlap between the gene set and genes highly expressed in MLL-AML (p=0.004).

### Example 11

Characterization of primary murine leukemias induced by transduction of GMP with an MLL-AF9 retrovirus. Histological evaluations of bone marrow, liver and spleen from a mouse with leukemia were studied. The bone marrow is completely replaced by large cells, with large nuclei and prominent nucleoli. The spleen is enlarged and engorged with cells similar to those in the bone marrow. The liver shows infiltration of leukemic cells into the parenchyma. FACS analysis of bone marrow demonstrates 93% of bone marrow cells are GFP positive. Most leukemic cells are Macl+/Grl+. Leukemic cells do not express the lymphoid markers B220 and CD3. Southern blot analysis was performed on genomic DNA isolated from 4 MLL-AF9 induced leukemias and demonstrates the leukemias are oligoclonal. The southern blot was performed using a probe for GFP.

### Example 12

### Methods Mice, Antibodies, and Sorting of Hematopoietic Progenitors

8 to 12 week C57B1/6 mice (Charles River Laboratories) were used as bone marrow donors and recipients. Myeloid hematopoietic progenitors were sorted as previously described^{u}. Please refer to details in supplemental methods. Since L-GMP expressed GFP we included anti-mouse Seal (Caltag) in the lineage mix, and used anti-mouse CD34-bio (BD) and Streptavidin-APC-Cy7 (Caltag) when we sorted this population from mice with AML. The CD48 antibody #13-0481-81 was obtained from eBioscience.

Retroviruses, Infections, Culture of Hematopoietic Progenitors

The MLL-AF9 cDNA was generously provided by Dr. Jay Hess and re-cloned into an MSCV based vector followed by IRES-GFP cassette (pMIG). Mef2C, HoxA9, HoxA7, HoxA10, and HoxA5 were amplified from total RNA isolated from L-GMP using primers specific for each gene. The resultant PCR product was cloned into pMSCV-puro (Clontech) and fully sequenced.

Ecotropic retroviral supernatants were produced by transient co-transfection of 293T cells as previously described³¹. ShRNA in lentiviral vectors were obtained from the RNAi Consortium, and viral particles generated by co-transfection of 293T cells with viral packaging plasmids. The Mef2c sequences were Mef2c-F6-GCCTCAGTGATACAGTATAAA (SEQ ID NO:1) and Mef2c-F7-CCATCAGTGAATCAAAGGATA (SEQ ID NO:2).

For GMP transduction, 5×10⁴ to 5×10⁵ GMP were incubated with retroviral supernatant including 20% PCS; 20 ng/ml mSCF (Peprotech) 10 ng/ml mIL-3 (Peprotech), 10 ng/ml mIL-6 (Peprotech), 1× penicillin-streptomycin (Invitrogen), 7 μg/ml polybrene (Sigma), and spun at 2000 rpm for 60 minutes at 37° C. 40 hours after the infection GMP were re-sorted for GFP PI-cells; then GFP⁺ cells were either injected (tail vein) into sub-lethally (600 RAD) irradiated recipient mice, collected in Trizol for RNA extraction, or plated in Methylcellulose media M3234 (Stem Cell Technologies) supplemented with 20 ng/ml mSCF (Peprotech) 10 ng/ml IL-3 (Peprotech), 10 ng/ml IL-6 (Peprotech), 10 ng/ml GM-C SF (Peprotech) and 1× penicillin/streptomycin. L-GMP sorted from leukemic mice were incubated with lentiviral vectors as described above for retroviruses and plated in methylcellulose media M3234 or liquid culture (Stem Cell Technologies) supplemented with 10 ng/ml IL-3 (Peprotech), and 1× penicillin/streptomycin (Gibco) with or without 2.5 mg/ml puromycin (Sigma).

Quantitative PCR

RNA was isolated from colonies derived from L-GMP and cDNA was generated using standard techniques. Real time PCR was performed using SYBR green detection reagents on a Sequence Detection System 7700 (Applied Biosystems) using primers for Mef2c and Gapdh. Ct values normalized against Gapdh as a housekeeping gene. Relative changes in concentrations were calculated according ΔΔCt method.

RNA Extraction, Amplification, and Microarrays

RNA was purified, amplified and labeled as described¹⁸. Detailed protocols for are available from the Broad Institute Molecular Pattern Recognition website (http://www.broad.mit.edu/mpr/publications/projects/leukemia/protocol.html

Data Analysis and Statistical Methods

After hybridization, the raw expression data was normalized as previously described to account for differences in chip intensities²³. Gene expression was then analyzed using the GeneCluster 2/GenePattern software or gene set enrichment analysis (GSEA) software (available at http://www.broad.mit.edu/tools/software.html). Details regarding GSEA can be found in supplemental methods and at the website. Hierarchical and K-means clustering were performed using the cluster software obtained from http://rana.lbl.gov/EisenSoftware.htm. The data were preprocessed using minimum and maximum expression values, a max/min filter, and max−min filter. The filters are shown for each signature. For comparisons of gene expression between two groups, the expression level correlated with a particular class was determined by comparing the means between the two groups using the signal-to-noise statistic23. To assess the murine signatures in human gene expression data we mapped the murine probes on the 430A 2.0 microarrays to probes on the human HU133A microarrays. First, we converted the murine probe set numbers to gene symbols using the latest Affymetrix annotation. Next we converted those gene symbols to (HU133A) probes using Affymetrix annotation. This new list of human probe sets was then used to assess human gene expression data. Stem cell frequency was calculated with the L-calc program from stem cell technologies.

Progenitor Sorting

Bone marrow was collected from both femur and tibia of C57Bl/6 donors by grinding the muscle free bones. Red blood cells were lysed on ice using red blood cells lysis buffer Puregene RBC Lysis Solution (cat#D-5001 Centra Systems). 5×10⁸ of nucleated bone marrow cells were incubated 40 min on ice with 100 u,l of each of the following lineage specific antibodies: Anti-mouse CD3 (Cat#RM3400, Caltag, CA), anti-mouse CD4 (Cat#MCD0400, Caltag, CA), anti-mouse CD8a (Cat#MCD0800, Caltag, CA), anti-mouse CD 19 (Cat#RM7700, Caltag, CA), anti-mouse B220 (CD45R) (Cat#RM2600, Caltag, CA), anti-mouse Grl (Cat#RM3000, Caltag, CA), anti-mouse TER119 (Cat#MTEROO, Caltag, CA) and anti-mouse CD127 (IL-7R) (Cat#14127181, Bioscience). After double washing in PBS the cell suspension was incubated with 50 u.l of secondary Goat-anti-rat F(ab)2 fragments labeled with Tri-Color (TC) (Cat#R40106, Caltag, CA). The cells were washed again and unbound Goat-anti-rat F(ab)2 fragments were blocked with 100 ng of rat-IgG (Cat#1-8015, Sigma, Mo.) for 10 min on ice. The cells were labeled with 50 fil of each Seal-bio (Cat#553334, BD, CA), Anti-mouse CD16/32 (Cat#553145, BD, CA); Anti-mouse CD117 (c-Kit) (Cat#553356, BD, CA); Anti-mouse CD34 (Cat#553733, BD, CA) for 30 min on ice, washed in PBS and Seal-bio antibody was then developed with 2 uJ of Streptavidine-APC-Cy7 (Cat#SA1014, Caltag, CA), dead cells were labeled with 7-AAD (Cat#A-1310, Molecular Probes, OR) for 15 min before the sorting. Mouse population enriched in hematopoietic stem cells (Lin⁻; 1L-7R⁻; Kit⁺; Scal⁺), and myeloid progenitors: CMP (IL-7R⁻ Scal⁻ Lin⁻ Kit⁺ CD34⁺ FcγRII/III^{hi}); GMP (IL⁻7R⁻Scal⁻ Lin⁻ Kit⁺ CD34⁺ FcγRII/III^{hi}) and MEP (1L-7R⁻ Scal⁻ Lin⁻ Kit⁺ CD34⁻ FcγRII/III¹⁰) were sorted using FACSAria multicolor cell sorter equipper with 488 and 635 nm lasers (BD, San Diego, Calif.).

Gene Set Enrichment Analysis (GSEA)

GSEA provides a general statistical method to test for the enrichment of sets of genes in expression data, and has been particularly useful in identifying molecular pathways at play in complex gene expression signatures, as we have recently reported²³ GSEA considers a priori defined Gene Sets, for example, genes in a signature such as the self-renewal associated signature or members of a pathway. It then provides a method to determine whether the members of these sets are over-represented at the top (or bottom) of a Gene List of markers which have been ordered by their correlation with a specific phenotype or class distinction, and produces a Gene Set-Gene List specific Enrichment Score (ES). The current implementation of GSEA is based on a Kolmogorov-Smirnov (KS) score to estimate the difference between the empirical cumulative distribution functions Pₕᵢₜ and Pₘᵢₛₛ, representing the fraction of genes from the of Gene Set G that are present (“hits”) or absent (“misses”) in the Gene List up to a given position:

$${P}_{hit} ( i ) = {\sum }_{j = 1}^{i} M ( j ) / {N}_{H}$$

$${P}_{miss} ( i ) = {\sum }_{j = 1}^{i} ( 1 - M ( j ) ) / {N}_{M}$$

The membership function M(J) takes the value 1 for a hit (i.e., the gene is in G) and 0 for a miss (i.e., the gene is not in G) at location j in the Gene List. NH (NM) is the total number of genes from G that are found (not found) in the Gene List. The difference between the two distributions is a “running” enrichment score S(i) and the maximum is the Maximum Enrichment Score (ES)

$$ES = {max}_{i = 1 , … , N} S ( i ) = {max}_{i = 1 , … , N} [ {P}_{hit} ( i ) - {P}_{miss} ( i ) ]$$

The significance of an observed ES(G) is obtained by permutation testing: reshuffling the phenotype labels and re-sorting the Gene List to determine how often an observed ES(G) occurs by chance. Statistical significance is computed by comparing the observed ES(G) with a histogram of ES(G,Π) values corresponding to the enrichment of the same Gene Set G but with reshuffled data according to a set of permutations Π=(1, . . . Π).

The running enrichment score is graphed vs. the gene # in gene list ordered based on the correlation of interest. Simply, the higher the ES score and the earlier in the ordered gene list the max ES score is obtained, the greater the enrichment of the gene set.

It is understood that the disclosed invention is not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a host cell” includes a plurality of such host cells, reference to “the miRNA” is a reference to one or more miRNAs and equivalents thereof known to those skilled in the art, and so forth.

Unless defined otherwise, all technical and scientific terms used herein have the same meanings as commonly understood by one of skill in the art to which the disclosed invention belongs. Although any methods and materials similar or equivalent to those described herein can be used in the practice or testing of the present invention, the preferred methods, devices, and materials are as described. Publications cited herein and the material for which they are cited are specifically incorporated by reference. Nothing herein is to be construed as an admission that the invention is not entitled to antedate such disclosure by virtue of prior invention.

TABLE 1
Mouse transplantation data

| | Transduction | | Estimated | | |
|---|---|---|---|---|---|
| | efficiency | Transplanted | transduced | Transplanted | |
| Cell population | (% GFP*) | cells (#) | cells (#) | mice (#) | #AML |
| Normal GMP | 30-36 | | 10000 | 7 | 7/7 (100%) |
| (MLL-AF9) | | | 5000 | 10 | 10/10 (100%) |
| | | | 1000 | 5 | 1/5 (20%) |
| | | | 100 | 5 | 0/5 (0%) |
| | | | 20 | 5 | 0/5 (0%) |
| leukemic BM | | 10000 | | 6 | 6/6 (100%) |
| | | 5000 | | 3 | 3/3 (100%) |
| | | 1000 | | 7 | 7/7 (100%) |
| | | 500 | | 2 | 2/2 (100%) |
| | | 100 | | 12 | 7/14 (50%) |
| | | 20 | | 5 | 0/5 (0%) |
| L-GMP | | 10000 | | 1 | 1/1 (100%) |
| | | 5000 | | 11 | 11/11 (100%) |
| | | 500 | | 7 | 7/7 (100%) |
| | | 100 | | 11 | 11/11 (100%) |
| | | 20 | | 22 | 22/22 (100%) |
| | | 4 | | 6 | 1/6 (17%) |
| Normal GMP | 40-50 | 20000-25000 | 10000 | 7 | 0 |
| (MSCV-GFP) | | | | | |
| AKLG Cells | | 100,000 | | 3 | 3/3 (100%) |
| | | 5.000 | | 3 | 0/3 (0%) |

TABLE 2

| | | | | GO Cellular |
|---|---|---|---|---|
| | | | Entrez | Component |
| Probe Set ID | Gene Title | Gene Symbol | Gene | Description |
| 1415677_at | dehydrogenase/reductase | Dhrs1 | 52585 | mitochondrial inner |
| | (SDR | | | membrane /// |
| | family) member 1 | | | endoplasmic |
| | | | | reticulum |
| 1415717_at | RIKEN cDNA | 4931406I20Rik | 66743 | — |
| | 4931406I20 gene | | | |
| 1415745_a_at | Down syndrome | Dscr3 | 13185 | retromer complex |
| | critical region | | | |
| | gene 3 | | | |
| 1415776_at | aldehyde | Aldh3a2 | 11671 | mitochondrial inner |
| | dehydrogenase | | | membrane /// |
| | family 3, | | | endoplasmic |
| | subfamily A2 | | | reticulum /// |
| | | | | microsome /// |
| | | | | membrane /// integral |
| | | | | to membrane /// |
| | | | | integral to membrane |
| 1415812_at | gelsolin | Gsn | 227753 | extracellular region /// |
| | | | | extracellular space /// |
| | | | | cytoplasm /// cytosol |
| | | | | /// cytoskeleton /// |
| | | | | actin cytoskeleton /// |
| | | | | actin cytoskeleton /// |
| | | | | lamellipodium /// |
| | | | | extracellular region /// |
| | | | | cytosol /// actin |
| | | | | cytoskeleton |
| 1415818_at | annexin A6 | Anxa6 | 11749 | perinuclear region |
| 1415886_at | SH2 domain | Sh2d3c | 27387 | intracellular |
| | containing 3C | | | |
| 1415983_at | lymphocyte | Lcp1 | 18826 | ruffle /// phagocytic |
| | cytosolic protein 1 | | | cup /// cytoplasm /// |
| | | | | cytosol /// actin |
| | | | | filament /// cytoplasm |
| 1416001_a_at | coactosin-like 1 | Cotl1 | 72042 | cellular_component /// |
| | (Dictyostelium) | | | intracellular /// |
| | | | | cytoskeleton |
| 1416013_at | phospholipase D | Pld3 | 18807 | membrane fraction /// |
| | family, member 3 | | | membrane /// integral |
| | | | | to membrane |
| 1416118_at | similar to mouse | LOC630539 | 630539 | intracellular /// |
| | RING finger 1 | | | membrane /// integral |
| | | | | to membrane |
| 1416148_at | lysosomal- | Laptm4b | 114128 | membrane /// integral |
| | associated protein | | | to membrane /// |
| | transmembrane | | | integral to membrane |
| | 4B | | | |
| 1416206_at | signal-induced | Sipa1 | 20469 | intracellular /// |
| | proliferation | | | nucleus /// nucleus /// |
| | associated gene 1 | | | nucleus |
| 1416246_a_at | coronin, actin | Coro1a | 12721 | lysosomal membrane |
| | binding protein | | | /// actin cytoskeleton |
| | 1A | | | |
| 1416253_at | cyclin-dependent | Cdkn2d | 12581 | nucleus /// cytoplasm |
| | kinase inhibitor | | | |
| | 2D (p19, inhibits | | | |
| | CDK4) | | | |
| 1416268_at | E26 avian | Ets2 | 23872 | intracellular /// |
| | leukemia | | | nucleus |
| | oncogene 2, 3′ | | | |
| | domain | | | |
| 1416401_at | CD82 antigen | Cd82 | 12521 | plasma membrane /// |
| | | | | integral to plasma |
| | | | | membrane /// |
| | | | | membrane /// integral |
| | | | | to membrane /// |
| | | | | integral to membrane |
| 1416614_at | EP300 interacting | Eid1 | 58521 | nucleus |
| | inhibitor of | | | |
| | differentiation 1 | | | |
| 1416635_at | sphingomyelin | Smpdl3a | 57319 | extracellular region /// |
| | phosphodiesterase, | | | extracellular space |
| | acid-like 3A | | | |
| 1416723_at | transcription | Tcf4 | 21413 | nucleus /// |
| | factor 4 | | | transcription factor |
| | | | | complex |
| 1416724_x_at | transcription | Tcf4 | 21413 | nucleus /// |
| | factor 4 | | | transcription factor |
| | | | | complex |
| 1416767_a_at | RIKEN cDNA | 1110003E01Rik | 68552 | membrane /// integral |
| | 1110003E01 gene | | | to membrane /// |
| | | | | integral to membrane |
| 1416768_at | RIKEN cDNA | 1110003E01Rik | 68552 | membrane /// integral |
| | 1110003E01 gene | | | to membrane /// |
| | | | | integral to membrane |
| 1416824_at | RIKEN cDNA | B230118H07Rik | 68170 | — |
| | B230118H07 | | | |
| | gene | | | |
| 1416861_at | signal transducing | Stam | 20844 | membrane |
| | adaptor molecule | | | |
| | (SH3 domain and | | | |
| | ITAM motif) 1 | | | |
| 1416935_at | transient receptor | Trpv2 | 22368 | intracellular /// plasma |
| | potential cation | | | membrane /// |
| | channel, | | | membrane /// integral |
| | subfamily V, | | | to membrane /// |
| | member 2 | | | membrane /// integral |
| | | | | to membrane |
| 1416966_at | solute carrier | Slc22a8 | 19879 | membrane /// integral |
| | family 22 | | | to membrane /// |
| | (organic anion | | | membrane /// integral |
| | transporter), | | | to membrane |
| | member 8 | | | |
| 1417109_at | tubulointerstitial | Tinagl | 94242 | cellular_component /// |
| | nephritis antigen- | | | extracellular region |
| | like | | | |
| 1417288_at | pleckstrin | Plekha2 | 83436 | nucleus /// membrane |
| | homology | | | |
| | domain- | | | |
| | containing, family A | | | |
| | (phosphoinositide | | | |
| | binding specific) | | | |
| | member 2 | | | |
| 1417302_at | REST corepressor 2 | Rcor2 | 104383 | nucleus /// nucleus |
| 1417371_at | pellino 1 | Peli1 | 67245 | — |
| 1417372_a_at | pellino 1 | Peli1 | 67245 | — |
| 1417378_at | immunoglobulin | Igsf4a | 54725 | plasma membrane /// |
| | superfamily, | | | intercellular junction |
| | member 4A | | | /// synaptic vesicle /// |
| | | | | integral to membrane |
| | | | | /// basolateral plasma |
| | | | | membrane /// axon /// |
| | | | | dendrite /// synapse /// |
| | | | | membrane |
| 1417379_at | IQ motif | Iqgap1 | 29875 | intracellular /// |
| | containing | | | cytoplasm /// |
| | GTPase | | | membrane /// actin |
| | activating protein 1 | | | filament |
| 1417423_at | glutamate | Grina | 66168 | integral to membrane |
| | receptor, | | | |
| | ionotropic, N- | | | |
| | methyl D- | | | |
| | asparate- | | | |
| | associated protein | | | |
| | 1 (glutamate | | | |
| | binding) | | | |
| 1417434_at | glycerol | Gpd2 | 14571 | extracellular space /// |
| | phosphate | | | mitochondrion /// |
| | dehydrogenase 2, | | | mitochondrion /// |
| | mitochondrial | | | mitochondrial inner |
| | | | | membrane /// |
| | | | | glycerol-3-phosphate |
| | | | | dehydrogenase |
| | | | | complex /// glycerol- |
| | | | | 3-phosphate |
| | | | | dehydrogenase |
| | | | | complex |
| 1417492_at | cathepsin B | Ctsb | 13030 | intracellular /// |
| | | | | mitochondrion /// |
| | | | | lysosome |
| 1417512_at | ecotropic viral | Evi5 | 14020 | nucleus |
| | integration site 5 | | | |
| 1417513_at | ecotropic viral | Evi5 | 14020 | nucleus |
| | integration site 5 | | | |
| 1417519_at | pleiomorphic | Plagl2 | 54711 | intracellular /// |
| | adenoma gene- | | | nucleus /// nucleus |
| | like 2 | | | |
| 1417533_a_at | integrin beta 5 | Itgb5 | 16419 | extracellular space /// |
| | | | | integrin complex /// |
| | | | | membrane /// integral |
| | | | | to membrane /// |
| | | | | integral to membrane |
| 1417534_at | integrin beta 5 | Itgb5 | 16419 | extracellular space /// |
| | | | | integrin complex /// |
| | | | | membrane /// integral |
| | | | | to membrane /// |
| | | | | integral to membrane |
| 1417551_at | ceroid | Cln3 | 12752 | Golgi membrane /// |
| | lipofuscinosis, | | | membrane fraction /// |
| | neuronal 3, | | | nucleus /// cytoplasm |
| | juvenile (Batten, | | | /// mitochondrion /// |
| | Spielmeyer-Vogt | | | lysosome /// lysosome |
| | disease) | | | /// early endosome /// |
| | | | | early endosome /// |
| | | | | late endosome /// |
| | | | | autophagic vacuole /// |
| | | | | endoplasmic |
| | | | | reticulum /// Golgi |
| | | | | apparatus /// Golgi |
| | | | | stack /// Golgi transface |
| | | | | /// plasma |
| | | | | membrane /// caveola |
| | | | | /// synaptic vesicle /// |
| | | | | membrane /// integral |
| | | | | to membrane /// |
| | | | | integral to membrane |
| | | | | /// integral to |
| | | | | endoplasmic |
| | | | | reticulum membrane |
| | | | | /// neuron projection |
| | | | | /// lipid raft /// |
| | | | | lysosome /// integral |
| | | | | to membrane |
| 1417604_at | calcium/calmodulin- | Camk1 | 52163 | nucleus /// calcium- |
| | dependent | | | and calmodulin- |
| | protein kinase I | | | dependent protein |
| | | | | kinase complex |
| 1417605_s_at | calcium/calmodulin- | Camk1 /// | 52163 | nucleus /// calcium- |
| | dependent | LOC669267 | /// | and calmodulin- |
| | protein kinase I /// | | 669267 | dependent protein |
| | similar to | | | kinase complex |
| | calcium/calmodulin- | | | |
| | dependent | | | |
| | protein kinase I | | | |
| 1417649_at | cyclin-dependent | Cdkn1c | 12577 | nucleus /// nucleus |
| | kinase inhibitor | | | |
| | 1C (P57) | | | |
| 1417707_at | RIKEN cDNA | B230342M21Rik | 100637 | — |
| | B230342M21 | | | |
| | gene | | | |
| 1417721_s_at | lysosomal- | Laptm5 /// | 16792 | lysosome /// |
| | associated protein | LOC669058 | /// | membrane /// integral |
| | transmembrane 5 | | 669058 | to membrane /// |
| | /// similar to | | | integral to membrane |
| | Lysosomal- | | | /// integral to plasma |
| | associated | | | membrane |
| | multitransmembrane | | | |
| | protein | | | |
| | (Retinoic acid- | | | |
| | inducible E3 | | | |
| | protein) | | | |
| 1417751_at | serine/threonine | Stk10 | 20868 | — |
| | kinase 10 | | | |
| 1417786_a_at | regulator of G- | Rgs19 | 56470 | membrane fraction /// |
| | protein signaling | | | Golgi apparatus /// |
| | 19 | | | heterotrimeric G- |
| | | | | protein complex /// |
| | | | | membrane |
| 1417801_a_at | protein tyrosine | Ppfibp2 | 19024 | — |
| | phosphatase, | | | |
| | receptor-type, F | | | |
| | interacting | | | |
| | protein, binding | | | |
| | protein 2 | | | |
| 1417840_at | RIKEN cDNA | 1500031L02Rik | 66994 | — |
| | 1500031L02 gene | | | |
| 1417896_at | tight junction | Tjp3 | 27375 | tight junction /// |
| | protein 3 | | | membrane /// tight |
| | | | | junction |
| 1417995_at | protein tyrosine | Ptpn22 | 19260 | — |
| | phosphatase, non- | | | |
| | receptor type 22 | | | |
| | (lymphoid) | | | |
| 1418025_at | basic helix-loop- | Bhlhb2 | 20893 | nucleus /// nucleus |
| | helix domain | | | |
| | containing, class | | | |
| | B2 | | | |
| 1418097_a_at | transmembrane | Tpte2 | 57914 | extracellular space /// |
| | phosphoinositide | | | membrane /// integral |
| | 3-phosphatase | | | to membrane |
| | and tensin | | | |
| | homolog 2 | | | |
| 1418196_at | telomerase | Tep1 | 21745 | chromosome, |
| | associated protein 1 | | | telomeric region /// |
| | | | | soluble fraction /// |
| | | | | nucleus /// |
| | | | | chromosome /// |
| | | | | telomerase |
| | | | | holoenzyme complex |
| | | | | /// cytoplasm /// |
| | | | | cytoplasm /// nuclear |
| | | | | matrix /// nuclear |
| | | | | matrix /// |
| | | | | ribonucleoprotein |
| | | | | complex |
| 1418201_at | pleckstrin | Plekhg2 | 101497 | intracellular |
| | homology domain | | | |
| | containing, family | | | |
| | G (with RhoGef | | | |
| | domain) member 2 | | | |
| 1418219_at | interleukin 15 | Il15 | 16168 | extracellular region /// |
| | | | | extracellular space /// |
| | | | | membrane fraction /// |
| | | | | cytoplasm /// |
| | | | | endosome /// Golgi |
| | | | | apparatus /// integral |
| | | | | to plasma membrane |
| 1418315_at | nuclear receptor | Nr5a1 | 26423 | nucleus /// nucleus |
| | subfamily 5, | | | |
| | group A, member 1 | | | |
| 1418344_at | transmembrane | Tmem8 | 60455 | extracellular space /// |
| | protein 8 (five | | | integral to plasma |
| | membrane- | | | membrane /// |
| | spanning | | | membrane /// integral |
| | domains) | | | to membrane /// |
| | | | | integral to plasma |
| | | | | membrane /// integral |
| | | | | to membrane /// |
| | | | | integral to plasma |
| | | | | membrane |
| 1418364_a_at | ferritin light chain 1 | Ftl1 | 14325 | — |
| 1418375_at | methyl-CpG | Mbd6 | 110962 | — |
| | binding domain | | | |
| | protein 6 | | | |
| 1418396_at | G-protein | Gpsm3 | 106512 | — |
| | signalling | | | |
| | modulator 3 | | | |
| | (AGS3-like, C. elegans) | | | |
| 1418472_at | aspartoacylase | Aspa | 11484 | — |
| | (aminoacylase) 2 | | | |
| 1418479_at | vacuolar protein | Vps54 | 245944 | cellular_component |
| | sorting 54 (yeast) | | | |
| 1418539_a_at | protein tyrosine | Ptpre | 19267 | extracellular space /// |
| | phosphatase, | | | plasma membrane /// |
| | receptor type, E | | | membrane /// integral |
| | | | | to membrane /// |
| | | | | soluble fraction /// |
| | | | | cytoplasm /// integral |
| | | | | to plasma membrane |
| | | | | /// integral to |
| | | | | membrane |
| 1418553_at | rho/rac guanine | Arhgef18 | 102098 | cellular_component /// |
| | nucleotide | | | intracellular |
| | exchange factor | | | |
| | (GEF) 18 | | | |
| 1418578_at | diacylglycerol | Dgka | 13139 | cytosol /// plasma |
| | kinase, alpha | | | membrane |
| 1418641_at | lymphocyte | Lcp2 | 16822 | — |
| | cytosolic protein 2 | | | |
| 1418686_at | 2′-5′ | Oas1c | 114643 | — |
| | oligoadenylate | | | |
| | synthetase 1C | | | |
| 1418812_a_at | BarH-like 1 | Barhl1 | 54422 | nucleus /// nucleus |
| | (Drosophila) | | | |
| 1418842_at | hematopoietic | Hcls1 | 15163 | nucleus /// DNA- |
| | cell specific Lyn | | | directed RNA |
| | substrate 1 | | | polymerase II, core |
| | | | | complex /// cytoplasm |
| 1418890_a_at | RAB3D, member | Rab3d | 19340 | intracellular /// |
| | RAS oncogene | | | membrane /// |
| | family | | | zymogen granule |
| 1418894_s_at | pre B-cell | Pbx2 | 18515 | nucleus /// nucleus |
| | leukemia | | | |
| | transcription | | | |
| | factor 2 | | | |
| 1418895_at | src family | Skap2 | 54353 | cytoplasm /// plasma |
| | associated | | | membrane |
| | phosphoprotein 2 | | | |
| 1419005_at | crystallin, beta B3 | Crybb3 | 12962 | cytoplasm |
| 1419103_a_at | abhydrolase | Abhd6 | 66082 | integral to membrane |
| | domain | | | |
| | containing 6 | | | |
| 1419120_at | lymphoblastomic | Lyl1 | 17095 | nucleus |
| | leukemia | | | |
| 1419125_at | protein tyrosine | Ptpn18 | 19253 | nucleus /// cytoplasm |
| | phosphatase, non- | | | /// nucleus |
| | receptor type 18 | | | |
| 1419193_a_at | glia maturation | Gmfg | 63986 | intracellular /// |
| | factor, gamma | | | intracellular |
| 1419198_at | chromobox | Cbx8 | 30951 | ubiquitin ligase |
| | homolog 8 | | | complex /// chromatin |
| | (Drosophila Pc | | | /// nucleus /// nucleus |
| | class) | | | /// PcG protein |
| | | | | complex |
| 1419206_at | CD37 antigen | Cd37 | 12493 | plasma membrane /// |
| | | | | integral to plasma |
| | | | | membrane /// |
| | | | | membrane /// integral |
| | | | | to membrane /// |
| | | | | integral to membrane |
| 1419224_at | cat eye syndrome | Cecr6 | 94047 | intracellular /// |
| | chromosome | | | integral to membrane |
| | region, candidate | | | |
| | 6 homolog | | | |
| | (human) | | | |
| 1419296_at | Rho GTPase | Arhgap4 | 171207 | cellular_component /// |
| | activating protein 4 | | | intracellular |
| 1419455_at | interleukin 10 | Il10rb | 16155 | integral to plasma |
| | receptor, beta | | | membrane /// |
| | | | | membrane /// integral |
| | | | | to membrane /// |
| | | | | plasma membrane /// |
| | | | | interleukin-28 |
| | | | | receptor complex |
| 1419631_at | Wiskott-Aldrich | Was | 22376 | cytoskeleton /// |
| | syndrome | | | vesicle membrane /// |
| | homolog (human) | | | actin cytoskeleton |
| 1419668_at | sarcoglycan, beta | Sgcb | 24051 | cytoskeleton /// |
| | (dystrophin- | | | integral to plasma |
| | associated | | | membrane /// |
| | glycoprotein) | | | sarcoglycan complex |
| | | | | /// membrane /// |
| | | | | integral to membrane |
| 1419742_at | RIKEN cDNA | 1700037H04Rik | 67326 | — |
| | 1700037H04 | | | |
| | gene | | | |
| 1419810_x_at | Rho GTPase | Arhgap9 | 216445 | cellular_component /// |
| | activating protein 9 | | | intracellular |
| 1419971_s_at | solute carrier | Slc35a5 | 74102 | Golgi membrane /// |
| | family 35, | | | integral to membrane |
| | member A5 | | | |
| 1420073_s_at | — | — | — | — |
| 1420091_s_at | microrchidia 3 | Morc3 | 338467 | — |
| 1420382_at | apolipoprotein | Apob48r | 171504 | cellular_component |
| | B48 receptor | | | |
| 1420453_at | crystallin, gamma S | Crygs | 12970 | cytoplasm |
| 1420464_s_at | paired-Ig-like | Pira1 /// Pira11 | 18722 | cellular_component /// |
| | receptor A1 /// | /// Pira2 /// Pira3 | /// | endoplasmic |
| | paired-Ig-like | /// Pira4 /// Pira6 | 18724 | reticulum /// |
| | receptor A11 /// | /// Lilrb3 /// | /// | endoplasmic |
| | paired-Ig-like | LOC619608 /// | 18725 | reticulum /// |
| | receptor A2 /// | LOC669449 /// | /// | membrane |
| | paired-Ig-like | LOC669458 /// | 18726 | |
| | receptor A3 /// | LOC675749 | /// | |
| | paired-Ig-like | | 18727 | |
| | receptor A4 /// | | /// | |
| | paired-Ig-like | | 18729 | |
| | receptor A6 /// | | /// | |
| | leukocyte | | 18733 | |
| | immunoglobulin- | | /// | |
| | like receptor, | | 619608 | |
| | subfamily B (with | | /// | |
| | TM and ITIM | | 669449 | |
| | domains), | | /// | |
| | member 3 /// | | 669458 | |
| | hypothetical | | /// | |
| | LOC619608 /// | | 675749 | |
| | similar to paired- | | | |
| | Ig-like receptor | | | |
| | A11 /// similar to | | | |
| | paired-Ig-like | | | |
| | receptor A11 /// | | | |
| | similar to paired- | | | |
| | Ig-like receptor | | | |
| | A1 | | | |
| 1420609_at | membrane- | 7-Mar | 57438 | cellular_component |
| | associated ring | | | |
| | finger (C3HC4) 7 | | | |
| 1420617_at | cytoplasmic | Cpeb4 | 67579 | — |
| | polyadenylation | | | |
| | element binding | | | |
| | protein 4 | | | |
| 1420704_at | colony | Csf2ra /// | 12982 | extracellular space /// |
| | stimulating factor | LOC671366 | /// | membrane /// integral |
| | 2 receptor, alpha, | | 671366 | to membrane /// |
| | low-affinity | | | integral to membrane |
| | (granulocyte- | | | |
| | macrophage) /// | | | |
| | similar to | | | |
| | Granulocyte- | | | |
| | macrophage | | | |
| | colony- | | | |
| | stimulating factor | | | |
| | receptor alpha | | | |
| | chain precursor | | | |
| | (GM-CSF-R- | | | |
| | alpha) (GMR) | | | |
| | (CD116 antigen) | | | |
| 1421023_at | phosphatidylinositol | Pik3c2a | 18704 | phosphoinositide 3- |
| | 3-kinase, C2 | | | kinase complex |
| | domain | | | |
| | containing, alpha | | | |
| | polypeptide | | | |
| 1421027_a_at | myocyte enhancer | Mef2c | 17260 | nucleus /// nucleus |
| | factor 2C | | | |
| 1421028_a_at | myocyte enhancer | Mef2c | 17260 | nucleus /// nucleus |
| | factor 2C | | | |
| 1421129_a_at | ATPase, Ca++ | Atp2a3 | 53313 | endoplasmic |
| | transporting, | | | reticulum /// |
| | ubiquitous | | | membrane /// integral |
| | | | | to membrane /// |
| | | | | endoplasmic |
| | | | | reticulum |
| 1421193_a_at | pre B-cell | Pbx3 | 18516 | nucleus /// nucleus /// |
| | leukemia | | | transcription factor |
| | transcription | | | complex /// |
| | factor 3 | | | transcription factor |
| | | | | complex |
| 1421392_a_at | baculoviral IAP | Birc3 | 11796 | intracellular |
| | repeat-containing 3 | | | |
| 1421525_a_at | baculoviral IAP | Birc1e | 17951 | intracellular |
| | repeat-containing | | | |
| | 1e | | | |
| 1421579_at | homeo box A9 | Hoxa9 | 15405 | nucleus /// |
| | | | | transcription factor |
| | | | | complex /// cytoplasm |
| | | | | /// nucleus |
| 1421863_at | vesicle-associated | Vamp1 | 22317 | membrane /// integral |
| | membrane protein 1 | | | to membrane /// |
| | | | | synaptosome /// |
| | | | | synapse |
| 1421923_at | SH3-domain | Sh3bp5 | 24056 | cytoplasm /// |
| | binding protein 5 | | | mitochondrion |
| | (BTK-associated) | | | |
| 1422124_a_at | protein tyrosine | Ptprc | 19264 | integral to plasma |
| | phosphatase, | | | membrane /// focal |
| | receptor type, C | | | adhesion /// external |
| | | | | side of plasma |
| | | | | membrane /// |
| | | | | membrane /// integral |
| | | | | to membrane /// focal |
| | | | | adhesion /// integral to |
| | | | | membrane |
| 1422467_at | palmitoyl-protein | Ppt1 | 19063 | extracellular space /// |
| | thioesterase 1 | | | extracellular space /// |
| | | | | lysosome /// lysosome |
| | | | | /// axon /// dendrite /// |
| | | | | cell soma /// |
| | | | | extracellular region /// |
| | | | | membrane fraction /// |
| | | | | nucleus /// lysosome |
| | | | | /// Golgi apparatus /// |
| | | | | cytosol /// synaptic |
| | | | | vesicle /// lysosome /// |
| | | | | axon /// lipid raft |
| 1422532_at | xeroderma | Xpc | 22591 | nucleus /// nucleus |
| | pigmentosum, | | | |
| | complementation | | | |
| | group C | | | |
| 1422568_at | nuclear | Ndel1 | 83431 | centrosome /// |
| | distribution gene | | | cytoskeleton /// |
| | E-like homolog 1 | | | microtubule /// |
| | (A. nidulans) | | | microtubule |
| | | | | associated complex /// |
| | | | | axon |
| 1422575_at | Max dimerization | Mxd4 /// | 17122 | nucleus /// |
| | protein 4 /// | LOC635153 | /// | transcription factor |
| | similar to Max | | 635153 | complex |
| | dimerization | | | |
| | protein 4 | | | |
| 1422653_at | centrosomal | Cep70 | 68121 | — |
| | protein 70 | | | |
| 1422721_at | tyrosine kinase, | Tnk1 | 83813 | cytoplasm /// cytosol |
| | non-receptor, 1 | | | /// membrane |
| 1422743_at | phosphorylase | Phka1 | 18679 | membrane |
| | kinase alpha 1 | | | |
| 1422760_at | peptidyl arginine | Padi4 /// | 18602 | nucleus |
| | deiminase, type | LOC670543 | /// | |
| | IV /// similar to | | 670543 | |
| | Protein-arginine | | | |
| | deiminase type IV | | | |
| | (Peptidylarginine | | | |
| | deiminase IV) | | | |
| 1422805_a_at | inhibitor of | Ing3 | 71777 | nucleus |
| | growth family, | | | |
| | member 3 | | | |
| 1422808_s_at | dedicator of cyto- | Dock2 | 94176 | cellular_component /// |
| | kinesis 2 | | | cytoskeleton /// |
| | | | | membrane |
| 1423002_at | phosphoprotein | Pag1 | 94212 | extracellular space /// |
| | associated with | | | plasma membrane /// |
| | glycosphingolipid | | | membrane /// integral |
| | microdomains 1 | | | to membrane /// lipid |
| | | | | raft |
| 1423182_at | tumor necrosis | Tnfrsf13b | 57916 | integral to plasma |
| | factor receptor | | | membrane /// external |
| | superfamily, | | | side of plasma |
| | member 13b | | | membrane /// |
| | | | | membrane /// integral |
| | | | | to membrane /// |
| | | | | integral to plasma |
| | | | | membrane /// integral |
| | | | | to membrane |
| 1423321_at | myeloid- | Myadm | 50918 | membrane /// integral |
| | associated | | | to membrane /// |
| | differentiation | | | integral to membrane |
| | marker | | | |
| 1423326_at | ectonucleoside | Entpd1 | 12495 | basal lamina /// |
| | triphosphate | | | plasma membrane /// |
| | diphosphohydrolase 1 | | | membrane /// integral |
| | | | | to membrane /// |
| | | | | integral to membrane |
| 1423383_a_at | oxysterol binding | Osbpl9 | 100273 | — |
| | protein-like 9 | | | |
| 1423414_at | prostaglandin- | Ptgs1 | 19224 | extracellular space /// |
| | endoperoxide | | | nucleus /// cytoplasm |
| | synthase 1 | | | /// microsome /// |
| | | | | membrane |
| 1423432_at | pleckstrin | Phip | 83946 | — |
| | homology domain | | | |
| | interacting | | | |
| | protein | | | |
| 1423461_a_at | ubiquitin-like 3 | Ubl3 | 24109 | cellular_component /// |
| | | | | membrane |
| 1423606_at | periostin, | Postn | 50706 | extracellular matrix |
| | osteoblast | | | (sensu Metazoa) /// |
| | specific factor | | | extracellular space /// |
| | | | | extracellular matrix |
| | | | | (sensu Metazoa) |
| 1423669_at | procollagen, type | Col1a1 | 12842 | extracellular matrix |
| | I, alpha 1 | | | (sensu Metazoa) /// |
| | | | | collagen /// collagen |
| | | | | type I /// extracellular |
| | | | | space /// cytoplasm |
| 1423760_at | CD44 antigen | Cd44 | 12505 | integral to plasma |
| | | | | membrane /// external |
| | | | | side of plasma |
| | | | | membrane /// |
| | | | | membrane /// integral |
| | | | | to membrane |
| 1423854_a_at | RAS-like, family | Rasl11b | 68939 | membrane |
| | 11, member B | | | |
| 1423914_at | RIKEN cDNA | C630004H02Rik | 217310 | membrane /// integral |
| | C630004H02 | | | to membrane |
| | gene | | | |
| 1423946_at | PDZ and LIM | Pdlim2 | 213019 | cytoskeleton |
| | domain 2 | | | |
| 1424027_at | paxillin | Pxn | 19303 | cytoskeleton /// focal |
| | | | | adhesion /// focal |
| | | | | adhesion /// |
| | | | | lamellipodium |
| 1424089_a_at | transcription | Tcf4 | 21413 | nucleus /// |
| | factor 4 | | | transcription factor |
| | | | | complex |
| 1424112_at | insulin-like | Igf2r | 16004 | extracellular space /// |
| | growth factor 2 | | | nucleus /// nuclear |
| | receptor | | | envelope lumen /// |
| | | | | lysosome /// |
| | | | | endosome /// |
| | | | | endosome /// integral |
| | | | | to plasma membrane |
| | | | | /// membrane /// |
| | | | | integral to membrane |
| | | | | /// trans-Golgi |
| | | | | network transport |
| | | | | vesicle /// trans-Golgi |
| | | | | network transport |
| | | | | vesicle /// integral to |
| | | | | membrane |
| 1424131_at | procollagen, type | Col6a3 /// | 12835 | extracellular matrix |
| | VI, alpha 3 /// | LOC674521 | /// | (sensu Metazoa) /// |
| | similar to alpha 3 | | 674521 | collagen /// |
| | type VI collagen | | | extracellular space /// |
| | isoform 1 | | | cytoplasm |
| | precursor | | | |
| 1424194_at | RCSD domain | Rcsd1 | 226594 | — |
| | containing 1 | | | |
| 1424249_a_at | Rho GTPase | Arhgap9 | 216445 | cellular_component /// |
| | activating protein 9 | | | intracellular |
| 1424250_a_at | Rho guanine | Arhgef3 | 71704 | cellular_component /// |
| | nucleotide | | | intracellular |
| | exchange factor | | | |
| | (GEF) 3 | | | |
| 1424256_at | retinol | Rdh12 | 77974 | extracellular space /// |
| | dehydrogenase 12 | | | intracellular /// |
| | | | | intracellular |
| 1424351_at | WAP four- | Wfdc2 | 67701 | extracellular space /// |
| | disulfide core | | | extracellular space |
| | domain 2 | | | |
| 1424383_at | transmembrane | Tmem51 | 214359 | membrane /// integral |
| | protein 51 | | | to membrane /// |
| | | | | integral to membrane |
| 1424384_a_at | zinc and ring | Znrf1 | 170737 | cellular_component |
| | finger 1 | | | |
| 1424523_at | engulfment and | Elmo1 | 140580 | cytoplasm /// |
| | cell motility 1, | | | cytoskeleton /// |
| | ced-12 homolog | | | plasma membrane /// |
| | (C. elegans) | | | membrane /// |
| | | | | cytoplasm /// plasma |
| | | | | membrane |
| 1424602_s_at | X-ray repair | Xrcc4 | 108138 | nucleus |
| | complementing | | | |
| | defective repair in | | | |
| | Chinese hamster | | | |
| | cells 4 | | | |
| 1424607_a_at | similar to novel | LOC545174 | 545174 | — |
| | protein | | | |
| 1424704_at | runt related | Runx2 | 12393 | nucleus /// cytoplasm |
| | transcription | | | /// nucleus |
| | factor 2 | | | |
| 1424778_at | receptor | Reep3 | 28193 | membrane /// integral |
| | accessory protein 3 | | | to membrane |
| 1424780_a_at | receptor | Reep3 | 28193 | membrane /// integral |
| | accessory protein 3 | | | to membrane |
| 1424781_at | receptor | Reep3 | 28193 | membrane /// integral |
| | accessory protein 3 | | | to membrane |
| 1424783_a_at | UDP | Ugt1a2 /// | 22236 | mitochondrial inner |
| | glucuronosyltrans | Ugt1a6a /// | /// | membrane /// |
| | ferase 1 family, | Ugt1a10 /// | 394430 | endoplasmic |
| | polypeptide A2 /// | Ugt1a7c /// | /// | reticulum /// |
| | UDP | Ugt1a5 /// | 394432 | microsome /// |
| | glucuronosyltransferase | Ugt1a9 /// | /// | membrane /// integral |
| | 1 family, | Ugt1a6b /// | 394433 | to membrane /// |
| | polypeptide A6A | Ugt1a1 | /// | integral to plasma |
| | /// UDP | | 394434 | membrane |
| | glycosyltransferase | | /// | |
| | 1 family, | | 394435 | |
| | polypeptide A10 | | /// | |
| | /// UDP | | 394436 | |
| | glucuronosyltransferase | | /// | |
| | 1 family, | | 94284 | |
| | polypeptide A7C | | | |
| | /// UDP | | | |
| | glucuronosyltransferase | | | |
| | 1 family, | | | |
| | polypeptide A5 /// | | | |
| | UDP | | | |
| | glucuronosyltransferase | | | |
| | 1 family, | | | |
| | polypeptide A9 /// | | | |
| | UDP | | | |
| | glucuronosyltransferase | | | |
| | 1 family, | | | |
| | polypeptide A6B | | | |
| | /// UDP | | | |
| | glucuronosyltransferase | | | |
| | 1 family, | | | |
| | polypeptide A1 | | | |
| 1424784_at | RIKEN cDNA | 1700029I01Rik | 433791 | intracellular |
| | 1700029I01 gene | /// LOC433791 | /// | |
| | /// similar to zinc | /// LOC666532 | 666532 | |
| | finger protein 665 | /// LOC671566 | /// | |
| | /// similar to zinc | | 671566 | |
| | finger protein 665 | | /// | |
| | /// similar to zinc | | 70005 | |
| | finger protein 665 | | | |
| 1424829_at | RIKEN cDNA | A830007P12Rik | 227612 | endoplasmic |
| | A830007P12 | | | reticulum |
| | gene | | | |
| 1424850_at | mitogen activated | Map3k1 /// | 26401 | — |
| | protein kinase | LOC670493 /// | /// | |
| | kinase kinase 1 /// | LOC674573 | 670493 | |
| | similar to | | /// | |
| | Mitogen-activated | | 674573 | |
| | protein kinase | | | |
| | kinase kinase 1 | | | |
| | (MAPK/ERK | | | |
| | kinase kinase 1) | | | |
| | (MEK kinase 1) | | | |
| | (MEKK 1) /// | | | |
| | similar to | | | |
| | Mitogen-activated | | | |
| | protein kinase | | | |
| | kinase kinase 1 | | | |
| | (MAPK/ERK | | | |
| | kinase kinase 1) | | | |
| | (MEK kinase 1) | | | |
| | (MEKK 1) | | | |
| 1424906_at | PQ loop repeat | Pqlc3 | 217430 | membrane /// integral |
| | containing | | | to membrane |
| 1424988_at | myosin regulatory | Mylip | 218203 | cytoplasm /// |
| | light chain | | | cytoskeleton /// |
| | interacting | | | membrane /// |
| | protein | | | cytoskeleton |
| 1424990_at | transmembrane | Tmem142a | 109305 | integral to plasma |
| | protein 142A | | | membrane /// |
| | | | | membrane /// integral |
| | | | | to membrane /// |
| | | | | integral to membrane |
| 1424996_at | CASP8 and | Cflar | 12633 | cytoplasm |
| | FADD-like | | | |
| | apoptosis | | | |
| | regulator | | | |
| 1425077_at | DnaJ (Hsp40) | Dnajc18 | 76594 | membrane /// integral |
| | homolog, | | | to membrane |
| | subfamily C, | | | |
| | member 18 | | | |
| 1425169_at | testis serine | Tessp2 | 235628 | — |
| | protease 2 | | | |
| 1425266_a_at | RAP1, GTP-GDP | Rap1gds1 | 229877 | — |
| | dissociation | | | |
| | stimulator 1 | | | |
| 1425469_a_at | Coronin, actin | Coro2a | 107684 | — |
| | binding protein | | | |
| | 2A | | | |
| 1425470_at | — | — | — | — |
| 1425471_x_at | — | — | — | — |
| 1425481_at | CCR4-NOT | Cnot61 | 231464 | cellular_component |
| | transcription | | | |
| | complex, subunit | | | |
| | 6-like | | | |
| 1425505_at | myosin, light | Mylk | 107589 | cytoskeleton |
| | polypeptide | | | |
| | kinase | | | |
| 1425506_at | myosin, light | Mylk | 107589 | cytoskeleton |
| | polypeptide | | | |
| | kinase | | | |
| 1425548_a_at | leukocyte specific | Lst1 | 16988 | cytoplasm /// Golgi |
| | transcript 1 | | | apparatus /// |
| | | | | membrane /// integral |
| | | | | to membrane /// |
| | | | | integral to membrane |
| | | | | /// integral to |
| | | | | membrane |
| 1425567_a_at | annexin A5 | Anxa5 | 11747 | intracellular /// |
| | | | | cytoplasm |
| 1425584_x_at | Coronin, actin | Coro2a | 107684 | — |
| | binding protein | | | |
| | 2A | | | |
| 1425704_at | cDNA sequence | BC022224 | 192970 | — |
| | BC022224 | | | |
| 1425736_at | CD37 antigen | Cd37 | 12493 | plasma membrane /// |
| | | | | integral to plasma |
| | | | | membrane /// |
| | | | | membrane /// integral |
| | | | | to membrane /// |
| | | | | integral to membrane |
| 1425747_at | RIKEN cDNA | 1110060D06Rik | 277133 | cellular_component |
| | 1110060D06 | /// Dock5 | /// | |
| | gene /// dedicator | | 68813 | |
| | of cytokinesis 5 | | | |
| 1426025_s_at | lysosomal- | Laptm5 | 16792 | lysosome /// |
| | associated protein | | | membrane /// integral |
| | transmembrane 5 | | | to membrane /// |
| | | | | integral to membrane |
| | | | | /// integral to plasma |
| | | | | membrane |
| 1426060_at | — | — | — | — |
| 1426098_a_at | calpastatin | Cast | 12380 | cellular_component |
| 1426100_a_at | thymidine kinase | Tk2 | 57813 | mitochondrion /// |
| | 2, mitochondrial | | | mitochondrial inner |
| | | | | membrane /// |
| | | | | mitochondrion |
| 1426204_a_at | opioid receptor- | Oprl1 | 18389 | integral to plasma |
| | like 1 | | | membrane /// |
| | | | | membrane /// integral |
| | | | | to membrane /// |
| | | | | integral to membrane |
| 1426210_x_at | poly (ADP- | Parp3 | 235587 | nucleus |
| | ribose) | | | |
| | polymerase | | | |
| | family, member 3 | | | |
| 1426239_s_at | arrestin, beta 2 | Arrb2 | 216869 | intracellular /// |
| | | | | nucleus |
| 1426249_at | adrenergic | Adrbk1 | 110355 | soluble fraction /// |
| | receptor kinase, | | | cytoplasm /// |
| | beta 1 | | | membrane |
| 1426261_s_at | UDP | Ugt1a2 /// | 22236 | mitochondrial inner |
| | glucuronosyltransferase | Ugt1a6a /// | /// | membrane /// |
| | 1 family, | Ugt1a10 /// | 394430 | endoplasmic |
| | polypeptide A2 /// | Ugt1a7c /// | /// | reticulum /// |
| | UDP | Ugt1a5 /// | 394432 | microsome /// |
| | glucuronosyltransferase | Ugt1a9 /// | /// | membrane /// integral |
| | 1 family, | Ugt1a6b /// | 394433 | to membrane /// |
| | polypeptide A6A | Ugt1a1 | /// | integral to plasma |
| | /// UDP | | 394434 | membrane |
| | glycosyltransferase | | /// | |
| | 1 family, | | 394435 | |
| | polypeptide A10 | | /// | |
| | /// UDP | | 394436 | |
| | glucuronosyltransferase | | /// | |
| | 1 family, | | 94284 | |
| | polypeptide A7C | | | |
| | /// UDP | | | |
| | glucuronosyltransferase | | | |
| | 1 family, | | | |
| | polypeptide A5 /// | | | |
| | UDP | | | |
| | glucuronosyltransferase | | | |
| | 1 family, | | | |
| | polypeptide A9 /// | | | |
| | UDP | | | |
| | glucuronosyltransferase | | | |
| | 1 family, | | | |
| | polypeptide A6B | | | |
| | /// UDP | | | |
| | glucuronosyltransferase | | | |
| | 1 family, | | | |
| | polypeptide A1 | | | |
| 1426272_at | limb region 1 | Lmbr1 | 56873 | membrane /// integral |
| | | | | to membrane |
| 1426299_at | RIKEN cDNA | 9130017C17Rik | 71607 | — |
| | 9130017C17 gene | | | |
| 1426400_a_at | calpain, small | Capns1 | 12336 | — |
| | subunit 1 | | | |
| 1426430_at | jagged 2 | Jag2 | 16450 | extracellular space /// |
| | | | | integral to plasma |
| | | | | membrane /// |
| | | | | membrane /// integral |
| | | | | to membrane /// |
| | | | | integral to membrane |
| 1426524_at | glucosamine-6- | Gnpda2 | 67980 | — |
| | phosphate | | | |
| | deaminase 2 | | | |
| 1426545_at | trinucleotide | Tnrc6b | 213988 | — |
| | repeat containing | | | |
| | 6b | | | |
| 1426586_at | solute carrier | Slc25a11 | 67863 | mitochondrion /// |
| | family 25 | | | mitochondrial inner |
| | (mitochondrial | | | membrane /// integral |
| | carrier | | | to plasma membrane |
| | oxoglutarate | | | /// membrane /// |
| | carrier), member | | | integral to membrane |
| | 11 | | | /// mitochondrial inner |
| | | | | membrane /// |
| | | | | mitochondrion |
| 1426587_a_at | signal transducer | Stat3 | 20848 | nucleus /// cytoplasm |
| | and activator of | | | /// plasma membrane |
| | transcription 3 | | | /// nucleus |
| 1426589_at | growth factor | Gab3 | 210710 | cellular_component |
| | receptor bound | | | |
| | protein 2- | | | |
| | associated protein 3 | | | |
| 1426597_s_at | expressed | C79267 | 212632 | — |
| | sequence C79267 | | | |
| 1426672_at | transmembrane | Tmem16k | 102566 | — |
| | protein 16K | | | |
| 1426686_s_at | mitogen activated | Map3k3 | 26406 | — |
| | protein kinase | | | |
| | kinase kinase 3 | | | |
| 1426716_at | tudor domain | Tdrd7 | 100121 | — |
| | containing 7 | | | |
| 1426725_s_at | E26 avian | Ets1 | 23871 | nucleus /// nucleus /// |
| | leukemia | | | nucleus |
| | oncogene 1, 5′ | | | |
| | domain | | | |
| 1426819_at | homeodomain | Hipk3 | 15259 | nucleus /// cytoplasm |
| | interacting | | | /// nucleus /// nucleus |
| | protein kinase 3 | | | |
| 1426833_at | eukaryotic | Eif4g3 | 230861 | eukaryotic translation |
| | translation | | | initiation factor 4F |
| | initiation factor 4 | | | complex /// membrane |
| | gamma, 3 | | | /// integral to |
| | | | | membrane |
| 1426883_at | expressed | AW491445 | 107375 | mitochondrial inner |
| | sequence | | | membrane /// |
| | AW491445 | | | membrane /// integral |
| | | | | to membrane /// |
| | | | | mitochondrial inner |
| | | | | membrane /// |
| | | | | membrane |
| 1426900_at | jumonji domain | Jmjd1c | 108829 | nucleus |
| | containing 1C | | | |
| 1426914_at | MARVEL | Marveld2 | 218518 | cellular_component /// |
| | (membrane- | | | membrane |
| | associating) | | | |
| | domain | | | |
| | containing 2 | | | |
| 1426926_at | phospholipase C, | Plcg2 | 234779 | — |
| | gamma 2 | | | |
| 1426953_at | high mobility | Hmgb2l1 | 70823 | chromatin /// nucleus |
| | group box 2-like 1 | | | |
| 1427001_s_at | hepatic nuclear | Hnf4a | 15378 | nucleus /// |
| | factor 4, alpha | | | transcription factor |
| | | | | complex /// nucleus |
| 1427155_at | FCH and double | Fchsd1 | 319262 | — |
| | SH3 domains 1 | | | |
| 1427242_at | DEAD (Asp-Glu- | Ddx4 | 13206 | nucleus /// cytoplasm |
| | Ala-Asp) box | | | |
| | polypeptide 4 | | | |
| 1427325_s_at | expressed | AI597013 | 100182 | — |
| | sequence | | | |
| | AI597013 | | | |
| 1427329_a_at | immunoglobulin | Igh-6 | 16019 | external side of |
| | heavy chain 6 | | | plasma membrane /// |
| | (heavy chain of | | | membrane /// integral |
| | IgM) | | | to membrane /// B cell |
| | | | | receptor complex /// |
| | | | | immunoglobulin |
| | | | | complex, circulating |
| | | | | /// MHC class I |
| | | | | protein complex /// |
| | | | | perinuclear region /// |
| | | | | multivesicular body |
| 1427334_s_at | RIKEN cDNA | 2810474O19Rik | 67246 | — |
| | 2810474O19 | | | |
| | gene | | | |
| 1427433_s_at | homeo box A3 | Hoxa3 | 15400 | nucleus /// |
| | | | | transcription factor |
| | | | | complex |
| 1427447_a_at | TRIO and F-actin | Triobp | 110253 | cellular_component /// |
| | binding protein | | | cytoskeleton /// actin |
| | | | | cytoskeleton /// actin |
| | | | | cytoskeleton |
| 1427535_s_at | expressed | AW822216 | 98733 | — |
| | sequence | | | |
| | AW822216 | | | |
| 1427689_a_at | TNFAIP3 | Tnip1 | 57783 | nucleus /// cytoplasm |
| | interacting | | | |
| | protein 1 | | | |
| 1427705_a_at | nuclear factor of | Nfkb1 | 18033 | nucleus /// nucleus /// |
| | kappa light chain | | | cytoplasm /// nucleus |
| | gene enhancer in | | | |
| | B-cells 1, p105 | | | |
| 1427717_at | CD80 antigen | Cd80 | 12519 | extracellular space /// |
| | | | | plasma membrane /// |
| | | | | membrane /// integral |
| | | | | to membrane /// |
| | | | | integral to membrane |
| 1427718_a_at | transformed | Mdm2 | 17246 | intracellular /// |
| | mouse 3T3 cell | | | nucleus /// |
| | double minute 2 | | | nucleoplasm /// |
| | | | | nucleolus /// |
| | | | | cytoplasm /// nucleus |
| 1427735_a_at | actin, alpha 1, | Actal | 11459 | stress fiber /// |
| | skeletal muscle | | | cytoskeleton /// |
| | | | | striated muscle thin |
| | | | | filament /// actin |
| | | | | filament /// actin |
| | | | | filament /// actin |
| | | | | cytoskeleton /// stress |
| | | | | fiber /// striated |
| | | | | muscle thin filament |
| | | | | /// actin filament |
| 1427892_at | myosin IG | Myo1g | 246177 | myosin complex |
| 1427918_a_at | ras homolog gene | Rhoq | 104215 | intracellular /// plasma |
| | family, member Q | | | membrane |
| 1427920_at | PHD finger | Phf19 | 74016 | — |
| | protein 19 | | | |
| 1427964_at | CKLF-like | Cmtm8 | 70031 | extracellular space /// |
| | MARVEL | | | membrane /// integral |
| | transmembrane | | | to membrane |
| | domain | | | |
| | containing 8 | | | |
| 1428015_at | DNA segment, | D12Wsu118e | 28115 | — |
| | Chr 12, Wayne | | | |
| | State University | | | |
| | 118, expressed | | | |
| 1428067_at | RAS-like, family | Rasl12 | 70784 | membrane |
| | 12 | | | |
| 1428217_at | RIKEN cDNA | 1600012H06Rik | 67912 | — |
| | 1600012H06 | | | |
| | gene | | | |
| 1428406_s_at | host cell factor | Hcfc1r1 | 353502 | — |
| | C1 regulator 1 | | | |
| | (XPO1- | | | |
| | dependent) | | | |
| 1428752_at | solute carrier | Slc5a11 | 233836 | membrane /// integral |
| | family 5 | | | to membrane /// |
| | (sodium/glucose | | | membrane |
| | cotransporter), | | | |
| | member 11 | | | |
| 1428784_at | Gem-interacting | Gmip | 78816 | intracellular |
| | protein | | | |
| 1429104_at | LIM domain | Limd2 | 67803 | — |
| | containing 2 | | | |
| 1429265_a_at | ring finger protein | Rnf130 | 59044 | membrane /// integral |
| | 130 | | | to membrane /// |
| | | | | integral to membrane |
| 1429569_a_at | coiled-coil | Ccdc46 | 76380 | — |
| | domain | | | |
| | containing 46 | | | |
| 1430549_at | blocked early in | Bet1l | 54399 | Golgi membrane /// |
| | transport 1 | | | membrane /// integral |
| | homolog (S. cerevisiae)- | | | to membrane /// |
| | like | | | integral to membrane |
| 1430676_at | procollagen, type | Col19a1 | 12823 | extracellular matrix |
| | XIX, alpha 1 | | | (sensu Metazoa) /// |
| | | | | collagen /// |
| | | | | extracellular space /// |
| | | | | cytoplasm |
| 1431299_a_at | RIKEN cDNA | 2310014H01Rik | 76448 | — |
| | 2310014H01 | | | |
| | gene | | | |
| 1431359_a_at | RIKEN cDNA | 1110007C09Rik | 68480 | intracellular /// |
| | 1110007C09 gene | | | nucleus /// cytosol |
| 1431475_a_at | homeo box A10 | Hoxa10 | 15395 | nucleus /// |
| | | | | transcription factor |
| | | | | complex |
| 1431914_at | phosphodiesterase | Pde3a | 54611 | membrane |
| | 3A, cGMP | | | |
| | inhibited | | | |
| 1432013_a_at | RIKEN cDNA | 2610016C23Rik | 71804 | cellular_component |
| | 2610016C23 gene | | | |
| 1432533_a_at | solute carrier | Slc35a2 | 22232 | Golgi membrane /// |
| | family 35 (UDP- | | | membrane /// integral |
| | galactose | | | to membrane /// Golgi |
| | transporter), | | | membrane |
| | member 2 | | | |
| 1433488_x_at | glucosamine (N- | Gns | 75612 | — |
| | acetyl)-6- | | | |
| | sulfatase | | | |
| 1433546_at | glucosamine (N- | Gns | 75612 | — |
| | acetyl)-6- | | | |
| | sulfatase | | | |
| 1433593_at | yippee-like 5 | Ypel5 | 383295 | cellular_component |
| | (Drosophila) | | | |
| 1433773_at | ribonucleotide | Rrm2b | 382985 | cellular_component /// |
| | reductase M2 B | | | nucleus |
| | (TP53 inducible) | | | |
| 1433829_a_at | heterogeneous | Hnrpa2b1 | 53379 | nucleus /// |
| | nuclear | | | spliceosome complex |
| | ribonucleoprotein | | | /// spliceosome |
| | A2/B1 | | | complex /// |
| | | | | ribonucleoprotein |
| | | | | complex /// |
| | | | | heterogeneous nuclear |
| | | | | ribonucleoprotein |
| | | | | complex |
| 1433830_at | heterogeneous | Hnrpa2b1 | 53379 | nucleus /// |
| | nuclear | | | spliceosome complex |
| | ribonucleoprotein | | | /// spliceosome |
| | A2/B1 | | | complex /// |
| | | | | ribonucleoprotein |
| | | | | complex /// |
| | | | | heterogeneous nuclear |
| | | | | ribonucleoprotein |
| | | | | complex |
| 1433842_at | leucine rich | Lrrfip1 | 16978 | nucleus |
| | repeat (in FLII) | | | |
| | interacting | | | |
| | protein 1 | | | |
| 1434037_s_at | p300/CBP- | Pcaf | 18519 | histone |
| | associated factor | | | acetyltransferase |
| | | | | complex /// |
| | | | | kinetochore |
| 1434062_at | RAB GTPase | Rabgap1l | 29809 | cellular_component |
| | activating protein | | | |
| | 1-like | | | |
| 1434148_at | transcription | Tcf4 | 21413 | nucleus /// |
| | factor 4 | | | transcription factor |
| | | | | complex |
| 1434149_at | transcription | Tcf4 | 21413 | nucleus /// |
| | factor 4 | | | transcription factor |
| | | | | complex |
| 1434199_at | gene model 323, | Gm323 | 210573 | — |
| | (NCBI) | | | |
| 1434206_s_at | protein | Ppp2r5c | 26931 | protein phosphatase |
| | phosphatase 2, | | | type 2A complex /// |
| | regulatory subunit | | | nucleus |
| | B (B56), gamma | | | |
| | isoform | | | |
| 1434379_at | Max dimerization | Mxd4 | 17122 | nucleus /// |
| | protein 4 | | | transcription factor |
| | | | | complex |
| 1434486_x_at | UDP-glucose | Ugp2 | 216558 | cytoplasm |
| | pyrophosphorylase 2 | | | |
| 1434517_at | WD repeat and | Wdfy2 | 268752 | — |
| | FYVE domain | | | |
| | containing 2 | | | |
| 1434653_at | PTK2 protein | Ptk2b | 19229 | cytoskeleton |
| | tyrosine kinase 2 | | | |
| | beta | | | |
| 1434796_at | vesicle-associated | Vamp4 | 53330 | Golgi membrane /// |
| | membrane protein 4 | | | lysosome /// |
| | | | | endosome /// |
| | | | | membrane /// integral |
| | | | | to membrane /// |
| | | | | integral to membrane |
| 1434882_at | Metadherin | Mtdh | 67154 | nucleus /// |
| | | | | endoplasmic |
| | | | | reticulum /// |
| | | | | membrane /// integral |
| | | | | to membrane |
| 1434900_at | MKL | Mkl1 | 223701 | nucleus /// cytoplasm |
| | (megakaryoblastic | | | /// nucleus |
| | leukemia)/myocardin- | | | |
| | like 1 | | | |
| 1434940_x_at | regulator of G- | Rgs19 | 56470 | membrane fraction /// |
| | protein signaling | | | Golgi apparatus /// |
| | 19 | | | heterotrimeric G- |
| | | | | protein complex /// |
| | | | | membrane |
| 1434998_at | IQ motif | Iqgap1 | 29875 | intracellular /// |
| | containing | | | cytoplasm /// |
| | GTPase | | | membrane /// actin |
| | activating protein 1 | | | filament |
| 1435174_at | rosbin, round | Rsbn1 | 229675 | nucleus |
| | spermatid basic | | | |
| | protein 1 | | | |
| 1435221_at | Adult male | — | — | — |
| | corpora | | | |
| | quadrigemina | | | |
| | cDNA, RIKEN | | | |
| | full-length | | | |
| | enriched library, | | | |
| | clone: B230341P20 | | | |
| | product: inferred: | | | |
| | forkhead box P1, | | | |
| | full insert | | | |
| | sequence | | | |
| 1435240_at | bromodomain | Baz2b | 407823 | — |
| | adjacent to zinc | | | |
| | finger domain, 2B | | | |
| 1435288_at | coronin, actin | Coro1a | 12721 | lysosomal membrane |
| | binding protein | | | /// actin cytoskeleton |
| | 1A | | | |
| 1435357_at | — | — | — | — |
| 1435666_at | microtubule | Mast3 | 546071 | — |
| | associated | | | |
| | serine/threonine | | | |
| | kinase 3 | | | |
| 1436097_x_at | Rho GTPase | Arhgap9 | 216445 | cellular_component /// |
| | activating protein 9 | | | intracellular |
| 1436180_at | DnaJ (Hsp40) | Dnajc5 | 13002 | membrane /// |
| | homolog, | | | membrane |
| | subfamily C, | | | |
| | member 5 | | | |
| 1436212_at | transmembrane | Tmem71 | 213068 | — |
| | protein 71 | | | |
| 1436297_a_at | glutamate | Grina | 66168 | integral to membrane |
| | receptor, | | | |
| | ionotropic, N- | | | |
| | methyl D- | | | |
| | asparate- | | | |
| | associated protein | | | |
| | 1 (glutamate | | | |
| | binding) | | | |
| 1436448_a_at | prostaglandin- | Ptgs1 | 19224 | extracellular space /// |
| | endoperoxide | | | nucleus /// cytoplasm |
| | synthase 1 | | | /// microsome /// |
| | | | | membrane |
| 1436510_a_at | leucine rich | Lrrfip2 | 71268 | — |
| | repeat (in FLII) | | | |
| | interacting | | | |
| | protein 2 | | | |
| 1436522_at | mitogen activated | Map3k3 | 26406 | — |
| | protein kinase | | | |
| | kinase kinase 3 | | | |
| 1436616_at | expressed | R74740 | 96938 | — |
| | sequence R74740 | | | |
| 1436766_at | LUC7-like 2 (S. cerevisiae) | Luc7l2 | 192196 | — |
| 1436767_at | LUC7-like 2 (S. cerevisiae) | Luc7l2 | 192196 | — |
| 1436861_at | interleukin 7 | Il7 | 16196 | extracellular region /// |
| | | | | extracellular region /// |
| | | | | extracellular space |
| 1436893_a_at | membrane- | 7-Mar | 57438 | cellular_component |
| | associated ring | | | |
| | finger (C3HC4) 7 | | | |
| 1436915_x_at | lysosomal- | Laptm4b | 114128 | membrane /// integral |
| | associated protein | | | to membrane /// |
| | transmembrane | | | integral to membrane |
| | 4B | | | |
| 1436953_at | WAS/WASL | Wipf1 | 215280 | actin cytoskeleton /// |
| | interacting | | | actin cytoskeleton /// |
| | protein family, | | | actin cytoskeleton |
| | member 1 | | | |
| 1436989_s_at | solute carrier | Slc12a6 | 107723 | membrane fraction /// |
| | family 12, | | | integral to plasma |
| | member 6 | | | membrane /// |
| | | | | membrane /// integral |
| | | | | to membrane /// |
| | | | | basolateral plasma |
| | | | | membrane /// apical |
| | | | | plasma membrane /// |
| | | | | axon /// integral to |
| | | | | membrane |
| 1436991_x_at | gelsolin | Gsn | 227753 | extracellular region /// |
| | | | | extracellular space /// |
| | | | | cytoplasm /// cytosol |
| | | | | /// cytoskeleton /// |
| | | | | actin cytoskeleton /// |
| | | | | actin cytoskeleton /// |
| | | | | lamellipodium /// |
| | | | | extracellular region /// |
| | | | | cytosol /// actin |
| | | | | cytoskeleton |
| 1437111_at | zinc finger CCCH | Zc3h12c | 244871 | — |
| | type containing | | | |
| | 12C | | | |
| 1437171_x_at | gelsolin | Gsn | 227753 | extracellular region /// |
| | | | | extracellular space /// |
| | | | | cytoplasm /// cytosol |
| | | | | /// cytoskeleton /// |
| | | | | actin cytoskeleton /// |
| | | | | actin cytoskeleton /// |
| | | | | lamellipodium /// |
| | | | | extracellular region /// |
| | | | | cytosol /// actin |
| | | | | cytoskeleton |
| 1437345_a_at | Bernardinelli- | Bscl2 | 14705 | endoplasmic |
| | Seip congenital | | | reticulum /// |
| | lipodystrophy 2 | | | membrane /// integral |
| | homolog (human) | | | to membrane /// |
| | | | | integral to |
| | | | | endoplasmic |
| | | | | reticulum membrane |
| | | | | /// integral to |
| | | | | membrane |
| 1437432_a_at | tripartite motif | Trim12 | 76681 | intracellular /// |
| | protein 12 | | | cytoplasm |
| 1437591_a_at | WD repeat | Wdr1 | 22388 | cytoskeleton /// actin |
| | domain 1 | | | cytoskeleton |
| 1437724_x_at | phosphatidylinositol | Pitpnm1 | 18739 | intracellular /// |
| | membrane- | | | endoplasmic |
| | associated 1 | | | reticulum /// |
| | | | | membrane /// |
| | | | | intracellular |
| 1437790_at | echinoderm | Eml5 | 319670 | cellular_component /// |
| | microtubule | | | microtubule |
| | associated protein | | | |
| | like 5 | | | |
| 1437791_s_at | echinoderm | Eml5 | 319670 | cellular_component /// |
| | microtubule | | | microtubule |
| | associated protein | | | |
| | like 5 | | | |
| 1438234_at | WD repeat | Wdr26 | 226757 | — |
| | domain 26 | | | |
| 1438365_x_at | lysosomal- | Laptm4b | 114128 | membrane /// integral |
| | associated protein | | | to membrane /// |
| | transmembrane | | | integral to membrane |
| | 4B | | | |
| 1438478_a_at | protein | Ppp3ca | 19055 | nucleus /// calcineurin |
| | phosphatase 3, | | | complex /// Z disc |
| | catalytic subunit, | | | |
| | alpha isoform | | | |
| 1438633_x_at | LIM and SH3 | Lasp1 | 16796 | cytoplasm /// |
| | protein 1 | | | cytoskeleton /// focal |
| | | | | adhesion /// cortical |
| | | | | actin cytoskeleton |
| 1438716_at | expressed | AI451617 | 209387 | intracellular |
| | sequence | | | |
| | AI451617 | | | |
| 1438759_x_at | — | — | — | — |
| 1438974_x_at | phosphatidylinositol | Pitpnm1 | 18739 | intracellular /// |
| | membrane- | | | endoplasmic |
| | associated 1 | | | reticulum /// |
| | | | | membrane /// |
| | | | | intracellular |
| 1439018_at | RIKEN cDNA | 6330505N24Rik | 229474 | — |
| | 6330505N24 | | | |
| | gene | | | |
| 1439264_x_at | LIM and SH3 | Lasp1 | 16796 | cytoplasm /// |
| | protein 1 | | | cytoskeleton /// focal |
| | | | | adhesion /// cortical |
| | | | | actin cytoskeleton |
| 1439380_x_at | GTL2, imprinted | Gtl2 | 17263 | — |
| | maternally | | | |
| | expressed | | | |
| | untranslated | | | |
| | mRNA | | | |
| 1439385_x_at | Solute carrier | Slc13a3 | 114644 | membrane fraction /// |
| | family 13 | | | membrane /// integral |
| | (sodium- | | | to membrane |
| | dependent | | | |
| | dicarboxylate | | | |
| | transporter), | | | |
| | member 3 | | | |
| 1439388_s_at | breast cancer anti- | Bcar1 | 12927 | ruffle /// membrane |
| | estrogen | | | fraction /// cytoplasm |
| | resistance 1 | | | /// focal adhesion /// |
| | | | | focal adhesion /// |
| | | | | lamellipodium /// |
| | | | | ruffle /// membrane |
| | | | | fraction /// cytoplasm |
| 1439389_s_at | myeloid- | Myadm | 50918 | membrane /// integral |
| | associated | | | to membrane /// |
| | differentiation | | | integral to membrane |
| | marker | | | |
| 1439441_x_at | large tumor | Lats2 | 50523 | spindle pole /// |
| | suppressor 2 | | | nucleus |
| 1447146_s_at | SLU7 splicing | Slu7 | 193116 | spliceosome complex |
| | factor homolog | | | /// nuclear speck /// |
| | (S. cerevisiae) | | | small nuclear |
| | | | | ribonucleoprotein |
| | | | | complex |
| 1448050_s_at | mitogen-activated | Map4k4 | 26921 | — |
| | protein kinase | | | |
| | kinase kinase | | | |
| | kinase 4 | | | |
| 1448121_at | WW domain | Wbp2 | 22378 | cellular_component |
| | binding protein 2 | | | |
| 1448182_a_at | CD24a antigen | Cd24a | 12484 | external side of |
| | | | | plasma membrane /// |
| | | | | membrane |
| 1448405_a_at | EP300 interacting | Eid1 | 58521 | nucleus |
| | inhibitor of | | | |
| | differentiation 1 | | | |
| 1448406_at | EP300 interacting | Eid1 | 58521 | nucleus |
| | inhibitor of | | | |
| | differentiation 1 | | | |
| 1448559_at | flotillin 1 | Flot1 | 14251 | caveola /// membrane |
| | | | | /// integral to |
| | | | | membrane /// flotillin |
| | | | | complex /// lipid raft |
| | | | | /// flotillin complex |
| 1448676_at | calcium/calmodulin- | Camk2b | 12323 | calcium- and |
| | dependent | | | calmodulin-dependent |
| | protein kinase II, | | | protein kinase |
| | beta | | | complex |
| 1448812_at | hippocalcin-like 1 | Hpcal1 | 53602 | — |
| 1448850_a_at | DnaJ (Hsp40) | Dnajc5 | 13002 | membrane /// |
| | homolog, | | | membrane |
| | subfamily C, | | | |
| | member 5 | | | |
| 1448926_at | homeo box A5 | Hoxa5 | 15402 | nucleus /// |
| | | | | transcription factor |
| | | | | complex |
| 1448985_at | dual specificity | Dusp22 | 105352 | cellular_component /// |
| | phosphatase 22 | | | nucleus |
| 1449043_at | N-acetyl | Naga | 17939 | lysosome |
| | galactosaminidase, | | | |
| | alpha | | | |
| 1449047_at | 2-hydroxyacyl- | Hacl1 | 56794 | peroxisome /// |
| | CoA lyase 1 | | | peroxisome /// |
| | | | | peroxisome |
| 1449076_x_at | acireductone | Adi1 | 104923 | nucleus /// nucleus /// |
| | dioxygenase 1 | | | cytoplasm /// plasma |
| | | | | membrane /// |
| | | | | membrane /// |
| | | | | cytoplasm /// plasma |
| | | | | membrane |
| 1449095_at | vacuolar protein | Vps54 | 245944 | cellular_component |
| | sorting 54 (yeast) | | | |
| 1449192_at | activating | Atf7ip | 54343 | nucleus /// |
| | transcription | | | transcription factor |
| | factor 7 | | | complex |
| | interacting | | | |
| | protein | | | |
| 1449216_at | integrin, alpha E, | Itgae | 16407 | extracellular space /// |
| | epithelial- | | | integrin complex /// |
| | associated | | | membrane /// integral |
| | | | | to membrane /// |
| | | | | integral to membrane |
| 1449222_at | Epstein-Barr | Ebi3 | 50498 | extracellular space /// |
| | virus induced | | | plasma membrane /// |
| | gene 3 | | | membrane /// |
| | | | | extracellular space /// |
| | | | | membrane |
| 1449360_at | colony | Csf2rb2 | 12984 | membrane /// integral |
| | stimulating factor | | | to membrane /// |
| | 2 receptor, beta 2, | | | integral to membrane |
| | low-affinity | | | |
| | (granulocyte- | | | |
| | macrophage) | | | |
| 1449419_at | dedicator of | Dock8 | 76088 | — |
| | cytokinesis 8 | | | |
| 1449420_at | phosphodiesterase | Pde1b | 18574 | — |
| | 1B, Ca2+- | | | |
| | calmodulin | | | |
| | dependent | | | |
| 1449580_s_at | histocompatibility | H2-DMb1 /// | 14999 | membrane /// integral |
| | 2, class II, locus | H2-DMb2 | /// | to membrane /// MHC |
| | Mb1 /// | | 15000 | class II protein |
| | histocompatibility | | | complex /// lysosome |
| | 2, class II, locus | | | /// lysosomal |
| | Mb2 | | | membrane /// late |
| | | | | endosome /// |
| | | | | multivesicular body |
| | | | | /// endosome |
| | | | | membrane /// integral |
| | | | | to membrane |
| 1449584_at | diacylglycerol | Dgkg | 110197 | — |
| | kinase, gamma | | | |
| 1449619_s_at | Rho GTPase | Arhgap9 | 216445 | cellular_component /// |
| | activating protein 9 | | | intracellular |
| 1449957_at | protein tyrosine | Ptprv | 13924 | extracellular space /// |
| | phosphatase, | | | membrane /// integral |
| | receptor type, V | | | to membrane /// |
| | | | | integral to membrane |
| 1450241_a_at | ecotropic viral | Evi2a | 14017 | membrane /// integral |
| | integration site 2a | | | to membrane /// |
| | | | | integral to membrane |
| 1450332_s_at | flavin containing | Fmo5 | 14263 | endoplasmic |
| | monooxygenase 5 | | | reticulum /// |
| | | | | microsome /// |
| | | | | membrane /// integral |
| | | | | to membrane /// |
| | | | | intrinsic to |
| | | | | endoplasmic |
| | | | | reticulum membrane |
| | | | | /// integral to |
| | | | | membrane |
| 1450350_a_at | Jun dimerization | Jundm2 | 81703 | cellular_component /// |
| | protein 2 | | | nucleus |
| 1450355_a_at | capping protein | Capg | 12332 | nucleus /// F-actin |
| | (actin filament), | | | capping protein |
| | gelsolin-like | | | complex |
| 1450513_at | CD33 antigen | Cd33 | 12489 | extracellular space /// |
| | | | | plasma membrane /// |
| | | | | membrane /// integral |
| | | | | to membrane /// |
| | | | | integral to membrane |
| 1450522_a_at | H1 histone | H1f0 | 14958 | nucleosome /// |
| | family, member 0 | | | nucleus /// |
| | | | | chromosome /// |
| | | | | nucleus |
| 1450629_at | LIM domain and | Lima1 | 65970 | stress fiber /// |
| | actin binding 1 | | | cytoskeleton /// focal |
| | | | | adhesion /// actin |
| | | | | cytoskeleton /// stress |
| | | | | fiber /// focal adhesion |
| 1450642_at | RIKEN cDNA | 3110001I20Rik | 70354 | — |
| | 3110001I20 gene | | | |
| 1450734_at | leucine zipper | Lztr2 | 89867 | cellular_component |
| | transcription | | | |
| | regulator 2 | | | |
| 1450754_at | similar to calcium | LOC669637 | 669637 | voltage-gated calcium |
| | channel, voltage- | | | channel complex /// |
| | dependent, alpha | | | membrane |
| | 2/delta subunit 2 | | | |
| 1450916_at | staufen (RNA | Stau2 | 29819 | intracellular /// |
| | binding protein) | | | nucleus /// |
| | homolog 2 | | | endoplasmic |
| | (Drosophila) | | | reticulum /// |
| | | | | microtubule /// |
| | | | | intracellular |
| 1450939_at | ectonucleoside | Entpd1 | 12495 | basal lamina /// |
| | triphosphate | | | plasma membrane /// |
| | diphosphohydrolase 1 | | | membrane /// integral |
| | | | | to membrane /// |
| | | | | integral to membrane |
| 1450964_a_at | oxysterol binding | Osbpl9 | 100273 | — |
| | protein-like 9 | | | |
| 1450966_at | carnitine O- | Crot | 74114 | peroxisome /// |
| | octanoyltransferase | | | peroxisome |
| 1450967_at | protein tyrosine | Ptplad2 | 66775 | integral to membrane |
| | phosphatase-like | | | |
| | A domain | | | |
| | containing 2 | | | |
| 1450992_a_at | myeloid ecotropic | Meis1 | 17268 | nucleus /// |
| | viral integration | | | transcription factor |
| | site 1 | | | complex /// nucleus |
| 1451021_a_at | Kruppel-like | Klf5 | 12224 | intracellular /// |
| | factor 5 | | | nucleus |
| 1451070_at | guanosine | Gdi1 | 14567 | cytoplasm |
| | diphosphate | | | |
| | (GDP) | | | |
| | dissociation | | | |
| | inhibitor 1 | | | |
| 1451097_at | vasodilator- | Vasp | 22323 | cytoskeleton /// focal |
| | stimulated | | | adhesion /// |
| | phosphoprotein | | | lamellipodium /// |
| | | | | filopodium |
| 1451114_at | CKLF-like | Cmtm6 | 67213 | extracellular space /// |
| | MARVEL | | | membrane /// integral |
| | transmembrane | | | to membrane |
| | domain | | | |
| | containing 6 | | | |
| 1451132_at | pre-B-cell | Pbxip1 | 229534 | — |
| | leukemia | | | |
| | transcription | | | |
| | factor interacting | | | |
| | protein 1 | | | |
| 1451186_at | interferon | Isg20l1 | 68048 | intracellular /// |
| | stimulated | | | nucleus |
| | exonuclease gene | | | |
| | 20-like 1 | | | |
| 1451202_at | RIKEN cDNA | C330007P06Rik | 77644 | — |
| | C330007P06 | | | |
| | gene | | | |
| 1451361_a_at | patatin-like | Pnpla7 | 241274 | — |
| | phospholipase | | | |
| | domain | | | |
| | containing 7 | | | |
| 1451362_at | RAB7, member | Rab7l1 | 226422 | membrane |
| | RAS oncogene | | | |
| | family-like 1 | | | |
| 1451388_a_at | ATPase, Class | Atp11b | 76295 | membrane /// integral |
| | VI, type 11B | | | to membrane /// |
| | | | | membrane /// integral |
| | | | | to membrane |
| 1451413_at | calpastatin | Cast | 12380 | cellular_component |
| 1451431_a_at | dysbindin | Dbndd2 | 52840 | cytoplasm |
| | (dystrobrevin | | | |
| | binding protein 1) | | | |
| | domain | | | |
| | containing 2 | | | |
| 1451461_a_at | aldolase 3, C | Aldoc | 11676 | mitochondrion |
| | isoform | | | |
| 1451465_at | ubiquitin-like 7 | Ubl7 | 69459 | membrane |
| | (bone marrow | | | |
| | stromal cell- | | | |
| | derived) | | | |
| 1451476_at | zinc finger, | Zdhhc8 | 27801 | membrane /// integral |
| | DHHC domain | | | to membrane /// |
| | containing 8 | | | perinuclear region |
| 1451491_at | RIKEN cDNA | 4930556P03Rik | 75284 | — |
| | 4930556P03 gene | | | |
| 1451506_at | myocyte enhancer | Mef2c | 17260 | nucleus /// nucleus |
| | factor 2C | | | |
| 1451507_at | myocyte enhancer | Mef2c | 17260 | nucleus |
| | factor 2C | | | |
| 1451622_at | LMBR1 domain | Lmbrd1 | 68421 | membrane /// integral |
| | containing 1 | | | to membrane /// |
| | | | | integral to membrane |
| 1451643_a_at | RAB4B, member | Rab4b | 19342 | intracellular /// |
| | RAS oncogene | | | membrane |
| | family | | | |
| 1451674_at | solute carrier | Slc12a5 | 57138 | plasma membrane /// |
| | family 12, | | | membrane /// integral |
| | member 5 | | | to membrane /// |
| | | | | integral to membrane |
| | | | | /// integral to |
| | | | | membrane |
| 1451719_at | cofactor required | Crsp6 | 234959 | mediator complex /// |
| | for Sp1 | | | transcription factor |
| | transcriptional | | | complex |
| | activation, | | | |
| | subunit 6 | | | |
| 1451723_at | CCR4-NOT | Cnot6l | 231464 | cellular_component |
| | transcription | | | |
| | complex, subunit | | | |
| | 6-like | | | |
| 1451793_at | kelch-like 24 | Klhl24 | 75785 | — |
| | (Drosophila) | | | |
| 1451821_a_at | nuclear antigen | Sp100 | 20684 | nucleus /// |
| | Sp100 | | | chromosome /// |
| | | | | membrane /// integral |
| | | | | to membrane |
| 1451969_s_at | poly (ADP- | Parp3 | 235587 | nucleus |
| | ribose) | | | |
| | polymerase | | | |
| | family, member 3 | | | |
| 1452016_at | arachidonate 5- | Alox5ap | 11690 | extracellular space /// |
| | lipoxygenase | | | membrane fraction /// |
| | activating protein | | | membrane /// integral |
| | | | | to membrane /// |
| | | | | integral to membrane |
| 1452035_at | procollagen, type | Col4a1 | 12826 | extracellular matrix |
| | IV, alpha 1 | | | (sensu Metazoa) /// |
| | | | | collagen /// collagen |
| | | | | type IV /// basement |
| | | | | membrane /// |
| | | | | extracellular space /// |
| | | | | cytoplasm /// |
| | | | | basement membrane |
| 1452050_at | calcium/calmodul | Camk1d | 227541 | nucleus /// cytoplasm |
| | in-dependent | | | /// calcium- and |
| | protein kinase ID | | | calmodulin-dependent |
| | | | | protein kinase |
| | | | | complex |
| 1452055_at | CTD (carboxy- | Ctdsp1 | 227292 | nucleus |
| | terminal domain, | | | |
| | RNA polymerase | | | |
| | II, polypeptide A) | | | |
| | small phosphatase 1 | | | |
| 1452056_s_at | protein | Ppp3ca | 19055 | nucleus /// calcineurin |
| | phosphatase 3, | | | complex /// Z disc |
| | catalytic subunit, | | | |
| | alpha isoform | | | |
| 1452063_at | RIKEN cDNA | 2410081M15Rik | 73680 | intracellular |
| | 2410081M15 | | | |
| | gene | | | |
| 1452079_s_at | DCUN1D1 | Dcun1d1 | 114893 | — |
| | DCN1, defective | | | |
| | in cullin | | | |
| | neddylation 1, | | | |
| | domain | | | |
| | containing 1 (S. cerevisiae) | | | |
| 1452080_a_at | DCUN1D1 | Dcun1d1 | 114893 | — |
| | DCN1, defective | | | |
| | in cullin | | | |
| | neddylation 1, | | | |
| | domain | | | |
| | containing 1 (S. cerevisiae) | | | |
| 1452123_s_at | FERM domain | Frmd4b | 232288 | cytoplasm /// |
| | containing 4B | | | cytoskeleton /// |
| | | | | membrane |
| 1452140_at | TBC1 domain | Tbc1d20 | 67231 | membrane /// integral |
| | family, member | | | to membrane |
| | 20 | | | |
| 1452163_at | E26 avian | Ets1 | 23871 | nucleus /// nucleus /// |
| | leukemia | | | nucleus |
| | oncogene 1, 5′ | | | |
| | domain | | | |
| 1452408_at | G protein-coupled | Gpr31c | 436440 | — |
| | receptor 31, | | | |
| | D17Leh66c | | | |
| | region | | | |
| 1452481_at | phospholipase C, | Plcb2 | 18796 | — |
| | beta 2 | | | |
| 1452565_x_at | hypothetical | LOC641050 | 641050 | viral capsid |
| | protein | | | |
| | LOC641050 | | | |
| 1452566_at | — | — | — | — |
| 1452784_at | integrin alpha V | Itgav | 16410 | integrin complex /// |
| | | | | membrane /// integral |
| | | | | to membrane |
| 1452823_at | glutathione S- | Gstk1 | 76263 | mitochondrion /// |
| | transferase kappa 1 | | | mitochondrion /// |
| | | | | mitochondrial inner |
| | | | | membrane /// |
| | | | | periplasmic space |
| | | | | (sensu Proteobacteria) |
| | | | | /// mitochondrion |
| 1452913_at | Purkinje cell | Pcp4l1 | 66425 | cellular_component |
| | protein 4-like 1 | | | |
| 1453576_at | Nipped-B | Nipb1 | 71175 | nucleus |
| | homolog | | | |
| | (Drosophila) | | | |
| 1454086_a_at | LIM domain only 2 | Lmo2 | 16909 | nucleus |
| 1454647_at | acyl-Coenzyme A | Acad11 | 102632 | peroxisome |
| | dehydrogenase | | | |
| | family, member | | | |
| | 11 | | | |
| 1455053_a_at | DCUN1D1 | Dcun1d1 | 114893 | — |
| | DCN1, defective | | | |
| | in cullin | | | |
| | neddylation 1, | | | |
| | domain | | | |
| | containing 1 (S. cerevisiae) | | | |
| 1455269_a_at | coronin, actin | Coro1a | 12721 | lysosomal membrane |
| | binding protein | | | /// actin cytoskeleton |
| | 1A | | | |
| 1455284_x_at | phosphatidylinositol | Pigx | 72084 | endoplasmic |
| | glycan anchor | | | reticulum /// |
| | biosynthesis, | | | membrane /// integral |
| | class X | | | to membrane |
| 1455470_x_at | LIM and SH3 | Lasp1 | 16796 | cytoplasm /// |
| | protein 1 | | | cytoskeleton /// focal |
| | | | | adhesion /// cortical |
| | | | | actin cytoskeleton |
| 1455626_at | homeo box A9 | Hoxa9 | 15405 | nucleus /// |
| | | | | transcription factor |
| | | | | complex /// cytoplasm |
| | | | | /// nucleus |
| 1455694_at | neurobeachin-like 2 | Nbeal2 | 235627 | — |
| 1455871_s_at | Tax1 (human T- | Tax1bp3 /// | 270106 | nucleus /// cytoplasm |
| | cell leukemia | Rpl13 | /// | /// intracellular /// |
| | virus type I) | | 76281 | cytosolic ribosome |
| | binding protein 3 | | | (sensu Eukaryota) /// |
| | /// ribosomal | | | ribosome /// |
| | protein L13 | | | ribonucleoprotein |
| | | | | complex /// nucleus |
| 1456133_x_at | integrin beta 5 | Itgb5 | 16419 | extracellular space /// |
| | | | | integrin complex /// |
| | | | | membrane /// integral |
| | | | | to membrane /// |
| | | | | integral to membrane |
| 1456195_x_at | integrin beta 5 | Itgb5 | 16419 | extracellular space /// |
| | | | | integrin complex /// |
| | | | | membrane /// integral |
| | | | | to membrane /// |
| | | | | integral to membrane |
| 1456309_x_at | LIM and SH3 | Lasp1 | 16796 | cytoplasm /// |
| | protein 1 | | | cytoskeleton /// focal |
| | | | | adhesion /// cortical |
| | | | | actin cytoskeleton |
| 1456377_x_at | LIM domain | Limd2 /// | 632329 | — |
| | containing 2 /// | LOC632329 | /// | |
| | similar to | | 67803 | |
| | epithelial protein | | | |
| | lost in neoplasm | | | |
| 1456427_at | glycoprotein Ib, | Gp1bb | 14724 | extracellular space /// |
| | beta polypeptide | | | integral to plasma |
| | | | | membrane /// |
| | | | | membrane /// integral |
| | | | | to membrane |
| 1456466_x_at | ataxin 10 | Atxn10 | 54138 | cellular_component /// |
| | | | | cytoplasm |
| 1456601_x_at | FXYD domain- | Fxyd2 | 11936 | membrane /// integral |
| | containing ion | | | to membrane /// |
| | transport | | | integral to membrane |
| | regulator 2 | | | |
| 1459903_at | sema domain, | Sema7a | 20361 | extracellular space /// |
| | immunoglobulin | | | membrane /// integral |
| | domain (Ig), and | | | to membrane /// |
| | GPI membrane | | | extracellular space |
| | anchor, | | | |
| | (semaphorin) 7A | | | |
| 1460188_at | protein tyrosine | Ptpn6 | 15170 | cytoplasm /// |
| | phosphatase, non- | | | cytoskeleton /// |
| | receptor type 6 | | | membrane |
| 1460194_at | phytanoyl-CoA | Phyh | 16922 | peroxisome /// |
| | hydroxylase | | | peroxisome |
| 1460222_at | SH3-domain | Sh3bp1 | 20401 | intracellular /// |
| | binding protein 1 | | | cytoplasm |
| 1460286_at | septin 6 | 6-Sep | 56526 | — |
| 1460344_at | RIKEN cDNA | 2310033F14Rik | 69555 | — |
| | 2310033F14 gene | | | |
| 1460674_at | progestin and | Paqr7 | 71904 | membrane /// integral |
| | adipoQ receptor | | | to membrane /// |
| | family member | | | integral to membrane |
| | VII | | | |
| 1460681_at | CEA-related cell | Ceacam1 | 26365 | membrane /// integral |
| | adhesion | | | to membrane /// |
| | molecule 1 | | | integral to membrane |
| | | | | /// cell surface |
| 1460700_at | signal transducer | Stat3 | 20848 | nucleus /// cytoplasm |
| | and activator of | | | /// plasma membrane |
| | transcription 3 | | | /// nucleus |
| 1460719_a_at | purinergic | P2rx1 | 18436 | integral to plasma |
| | receptor P2X, | | | membrane /// |
| | ligand-gated ion | | | membrane /// integral |
| | channel, 1 | | | to membrane /// |
| | | | | integral to membrane |
| AFFX-b- | actin, beta, | Actb | 11461 | soluble fraction /// |
| ActinMur/M1 | cytoplasmic | | | cytosol /// |
| 2481_M_at | | | | cytoskeleton |

### REFERENCES

1. Reya, T., Morrison, S. J., Clarke, M. F. & Weissman, I. L. Stem cells, cancer, and cancer stem cells. Nature 414, 105-11 (2001).
2. Warner, J. K., Wang, J. C., Hope, K. J., Jin, L. & Dick, J. E. Concepts of human leukemic development. Oncogene 23, 7164-77 (2004).
3. Al-Hajj, M. & Clarke, M. F. Self-renewal and solid tumor stem cells. Oncogene 23, (2004).
4. Shivdasani, R. A. & Orkin, S. H. The transcriptional control of hematopoiesis. Blood 87, 4025-39 (1996).
5. Weissman, I. L., Anderson, D. J. & Gage, F. Stem and progenitor cells: origins, phenotypes, lineage commitments, and transdifferentiations. Annu Rev Cell Dev Biol 17, 387-403 (2001).
6. Lapidot, T. et al. A cell initiating human acute myeloid leukaemia after transplantation into SC1D mice. Nature 367, 645-8 (1994).
7. Bonnet, D. & Dick, I. E. Human acute myeloid leukemia is organized as a hierarchy that originates from a primitive hematopoietic cell. Nat Med 3, 730-7 (1997).
8. Huntly, B. J. et al. MOZ-TIF2, but not BCR-ABL, confers properties of leukemic stem cells to committed murine hematopoietic progenitors. Cancer Cell 6, 587-96 (2004).
9. Cozzio, A. et al. Similar MLL-associated leukemias arising from self-renewing stem cells and short-lived myeloid progenitors. Genes Dev 17, 3029-35 (2003).
10. Jamieson, C. H. et al. Granulocyte-macrophage progenitors as candidate leukemic stem cells in blast-crisis CML. N Engl J Medl Sl, 657-67 (2004).
11. So, C. W. et al. MLL-GAS7 transforms multipotent hematopoietic progenitors and induces mixed lineage leukemias in mice. Cancer Cell 3, 161-71 (2003).
12. Akashi, K., Traver, D., Miyamoto, T. & Weissman, I. L. A clonogenic common myeloid progenitor that gives rise to all myeloid lineages. Nature 404, 193-7 (2000).
13. Park, I. K. et al. Differential gene expression profiling of adult murine hematopoietic stem cells. Blood 99, 488-98 (2002).
14. Akashi, K. et al. Transcriptional accessibility for genes of multiple tissues and hematopoietic lineages is hierarchically controlled during early hematopoiesis. Blood 101, 383-9 (2003).
15. Kumar, A. R. et al. IIoxa9 influences the phenotype but not the incidence of M11-AF9 fusion gene leukemia. Blood 103, 1823-8 (2004).
16. Lavau, C., Szilvassy, S. J., Slany, R. & Cleary, M. L. Immortalization and leukemic transformation of a myelomonocytic precursor by retrovirally transduced HRX-ENL. Embo, 716, 4226-37 (1997).
17. Corral, J. et al. An M11-AF9 fusion gene made by homologous recombination causes acute leukemia in chimeric mice: a method to create fusion oncogenes. Cell 85, 853-61 (1996).
18. Wang, J. et al. Conditional MLL-CBP targets GMP and models therapy-related myeloproliferative disease. Embo J 24, 368-81 (2005).
19. Drynan, L. F. et al. Mil fusions generated by Cre-loxP-mediated de novo translocations can induce lineage reassignment in tumorigenesis. Embo J 24, 3136-46 (2005).
20. Ramalho-Santos, M., Yoon, S., Matsuzaki, Y., Mulligan, R. C. & Melton, D. A. “Sternness”: transcriptional profiling of embryonic and adult stem cells. Science 298, 597-600 (2002).
21. Zeisig, B. B. et al. Hoxa9 and Meis1 are key targets for MLL-ENL-mediated cellular immortalization. Mol Cell Biol 24, 617-28 (2004).
22. Ayton, P. M. & Cleary, M. L. Transformation of myeloid progenitors by MLL oncoproteins is dependent on Hoxa7 and Hoxa9. Genes Dev 17, 2298-307 (2003).
23. Armstrong, S. A. et al. MLL translocations specify a distinct gene expression profile that distinguishes a unique leukemia. Nat Genet 30, 41-7 (2002).
24. So, C. W., Karsunky, H., Wong, P., Weissman, I. L. & Cleary, M. L. Leukemic transformation of hematopoietic progenitors by MLL-GAS7 in the absence of Hoxa7 or Hoxa9. Blood 103, 3192-9 (2004).
25. Kiel, M. J., Yilmaz, O. H., Iwashita, T., Terhorst, C. & Morrison, S. J. SLAM family receptors distinguish hematopoietic stem and progenitor cells and reveal endothelial niches for stem cells. Cell 121, 1109-21 (2005).
26. Subramanian, A. et al. From the Cover: Gene set enrichment analysis: A knowledge-based approach for interpreting genome-wide expression profiles. Proc Natl Acad Sci USA 102, 15545-50 (2005).
27. Abramovich, C. & Humphries, R. K. Hox regulation of normal and leukemic hematopoietic stem cells. Curr Opin Hematol 12, 210-6 (2005).
28. Milne, T. A. et al. From The Cover: MLL associates specifically with a subset of transcriptionally active target genes. Proc Natl Acad Sci USA 102, 14765-70 (2005).
29. Guenther, M. G. et al. Global and Hox-specific roles for the MLL1 methyltransferase. Proc Natl Acad Sci USA 102, 8603-8 (2005).
30. Du, Y., Spence, S. E., Jenkins, N. A. & Copeland, N. G. Cooperating cancer-gene identification through oncogenic-retrovirus-induced insertional mutagenesis. Blood 106, 2498-505 (2005).
31. Ernst, P., Mabon, M., Davidson, A. J., Zon, L. I. & Korsmeyer, S. J. An M11-dependent Hox program drives hematopoietic progenitor expansion. Curr Biol 14, 2063-9 (2004).
32. Ross, M. E. et al. Gene expression profiling of pediatric acute myelogenous leukemia. Blood 104, 3679-87 (2004).

Those skilled in the art will recognize, or be able to ascertain using no more than routine experimentation, many equivalents to the specific embodiments of the invention described herein. Such equivalents are intended to be encompassed by the following claims.

What is claimed is:

1. A method for diagnosing a leukemia in a first tissue sample of an individual, comprising the steps of (a) determining the expression profile of one or more self-renewal associated signature genes of cancer-like progenitor cells in the first tissue sample, and (b) comparing the pattern or level of expression profile observed with the pattern or level of expression of the same genes in a second tissue sample comprising committed progenitor cells, wherein increased expression of the one or more self-renewal associated signature genes in the first tissue sample indicates leukemia.

2. The method of claim 1 wherein the first and second tissue are selected from the group consisting of epithelial tissue, connective tissue, osseous tissue, vascular tissue, blood, muscle tissue, nervous tissue, and cartilage.

3. The method of claim 1 wherein the second tissue sample is the same as the first tissue sample.

4. The method of claim 1, wherein the first tissue sample and the second tissue sample are from different individuals.

5. The method of claim 1 wherein committed progenitor cells are selected from the group consisting of granulocyte-macrophage progenitors, common myeloid progenitors (CMP), and megakaryocyte erythroid progenitors (MEP).

6. The method of claim 1 wherein the one or more self-renewal associated signature genes are selected from the group consisting of the genes in TABLE 2.

7. The method of claim 6 wherein the one or more self-renewal associated signature genes are selected from the group consisting of HOXA9, HOXA10, MEF2c, HOXA5, Meis1, ITF2, MYLK, RUNX2, PELI1, LAPTM4b, and STAU2.

8. The method of claim 7 wherein the one or more of the genes is expressed at higher levels in the first tissue sample than in the corresponding one or more genes in the second tissue sample.

9. A method for targeted therapeutic treatment of leukemia or cancer cells, comprising administering to a patient in need thereof an effective amount of a therapeutic agent that targets one or more self-renewal signature genes expressed in the leukemia or cancer cells.

10. A method for targeted therapeutic treatment of leukemia or cancer cells, comprising administering to a patient in need thereof an effective amount of a therapeutic agent that targets one or more self-renewal signature gene products expressed in the leukemia or cancer cells.

11. The method of claim 10, wherein the therapeutic comprises a drug conjugated to an immunoglobulin or aptamer that specifically recognizes an epitope on a protein encoded by the one or more self-renewal signature genes.

12. The method of claim 9, wherein the therapeutic reduces in vivo expression of the one or more self-renewal signature genes.

13. The method of claim 12, wherein the therapeutic is a polynucleotide capable of binding to and reducing the expression of a nucleic acid encoding one or more of the self-renewal signature genes.

14. The method of claim 12, wherein the therapeutic is an effective amount of a siNA complementary to a target 3′UTR mRNA encoded by one or more self-renewal signature genes.

15. The method of claim 14, wherein the siNA is a miRNA.

16. The method of claim 15, wherein the miRNA directs RNA interference of the target 3′UTR mRNA encoded by one or more self-renewal signature genes.

17. The method of claim 16, wherein the RNA interference results in the target mRNA being degraded.

18. The method of claim 16, wherein the RNA interference results in the target mRNA being translationally repressed.

19. The method of claim 10, wherein the gene expression product is RNA.

20. A transformed cell line wherein the cell line expresses an MLL-AF9 fusion protein.

21. The transformed cell line of claim 20, wherein the cell line is a committed progenitor.

22. The transformed cell line of claim 21, wherein the committed progenitor is selected from the group consisting of granulocyte-macrophage progenitors (GMP); common myeloid progenitors (CMP), and megakaryocyte erythroid progenitors (MEP).

23. The method of claim 14, wherein the siNA is administered by a route selected from the group consisting of oral, intravenous, intramuscular, and intrapulmonary.

24. A method for detecting the presence of leukemia stem cells in a tissue sample, comprising reacting the first tissue sample with one or more antibodies that specifically bind to one or more gene products of the self-renewal associated signature genes, wherein detecting the antibody-gene product complex indicates the presence of leukemia stem cells.

25. The method of claim 24, wherein the antibody is specific for EPHA7.

26. The method of claim 23, further comprising isolating the leukemia stem cells.

27. The method of claim 26, wherein the leukemia stem cells are isolated by immunoprecipitation.
